(19) United States
(12) Patent Application Publication
(10) Pub. No.: US 2006/0223743 A1
(43) Pub. Date: Oct. 5, 2006

(54) Metabolites of cyclosporin analogs

(76) Inventors: Mark Abel, Edmonton (CA); Robert T. Foster, Edmonton (CA); Derrick G. Freitag, Edmonton (CA); Daniel J. Trepanier, Edmonton (CA); Shin Sugiyama, Edmonton (CA); Seetharaman Jayaraman, Edmonton (CA); Randall W. Yatscoff, Edmonton (CA)

Correspondence Address: FISH & RICHARDSON P.C., PO BOX 1022, MINNEAPOLIS, MN, 55440-1022, US

(21) Appl. No.: 11/313,239

(22) Filed: Dec. 19, 2005

Related U.S. Application Data
(60) Provisional application No. 60/637,392, filed on Dec. 17, 2004.

(51) Int. Cl. A61K 38/13; C07K 7/64

(52) U.S. Cl. 514/7; 514/11; 530/317

(57) ABSTRACT

Isolated metabolites of the cyclosporine analog ISA247 are disclosed, including in vitro methods for their preparation. The metabolites comprise a chemical modification of ISA247, wherein the modification is at least one reaction selected from the group consisting of hydroxylation, N-demethylation, diol formation, epoxide formation, and intramolecular cyclization phosphorylation, sulfation, glucuronide formation and glycosylation. Methods of preparation include semi-synthetic methods, wherein metabolites of ISA247 are produced from the microsomal extracts of animal liver cells, or from cultures using microorganisms, and completely synthetic methods, such as chemically modifying the parent compound or isolated metabolites using organic synthetic methods.

### CROSS REFERENCE TO RELATED APPLICATIONS

This application claims the benefit of U.S. Provisional Patent Application Serial No. 60/637,392, filed Dec. 17, 2004, the entire teachings of which are incorporated herein by reference.

### FIELD OF THE INVENTION

The present invention relates to isolated metabolites of ISA247 or ISA_{TX}247, a derivative of cyclosporine A. The present invention also relates to methods of making and analyzing isolated metabolites of ISA247.

### REFERENCES

U.S. Pat. No. 6,605,593

U.S. Pat. No. 6,613,739

US 2003/0212249.

International Publication No. WO 99/18120

International Publication No. WO 03/033527

International Publication No. WO 2003/033526

International Publication No. WO 2003/033527

Brown, H. C. et al., J. Am. Chem. Soc. vol 110, p 1535 (1988).

Christians, et al. “Cyclosporine Metabolism in Transplant Patients;” Pharmac. Ther. vol 57, pp 291-345 (1993).

Eberle M. K. and F. Nuninger, “Synthesis of the main metabolite (OL-17) of cyclosporin A,” J. Org. Chem. Vol. 57, No. 9, pp. 2689-2691 (1992).

Hartman, N. R. and I. Jardine “Mass Spectrometric Analysis of Cyclosporine Metabolites,” Biomed. Environ. Mass Spectrom. Vol. 13, pp. 361-372 (1986).

Hu et al., J. Org. Chem. vol 63, p 8843 (1998).

Johnson, R. A., and Sharpless, K. B.; Catalytic Asymmetric Synthesis: Edited by I. Ojima; VCH Publishers: New York; 1993; p. 103

Keown, P. A., “Molecular and Clinical Therapeutics of Cyclosporine in Transplantation” in Immunosuppression in Transplantation (Blackwell Science, Malden Mass., 1999), pp. 1-12.

Marshall, J. A. Chem Rev. vol 96, p 31(1996).

Barrett, A. G. M. et al., J. Org. Chem. vol 56, p 5243 (1991).

Sharpless, K. B. et al., J. Org. Chem. vol 57, p 2768 (1992).

Wenger, R. M. “Synthesis of Cyclosporine and Analogs: Structural Requirements for Immunosuppressive Activity,” Angew Chem Int. Ed. Engl. Vol. 24, No. 2, pp. 77-138. (1985).

### BACKGROUND

Cyclosporins are members of a class of cyclic polypeptides having potent immunosuppressant activity. At least some of these compounds, such as Cyclosporin A, are produced by the species Tolypocladium inflatum Gams as secondary metabolites. As an immunosuppressent agent, cyclosporin has been demonstrated to suppress humoral immunity and cell-mediated immune reactions, such as allograft rejection, delayed hypersensitivity, experimental allergic encephalomyelitis, Freund's adjuvant arthritis and graft vs. host disease. It is used for the prophylaxis of organ rejection in organ transplants; for the treatment of rheumatoid arthritis; and for the treatment of psoriasis.

Although a number of compounds in the cyclosporin family are known, cyclosporine A is perhaps the most widely used medically. The immunosuppressive effects of cyclosporine A are related to the inhibition of T-cell mediated activation events. Immunosuppression is accomplished by the binding of cyclosporin to a ubiquitous intracellular protein called cyclophilin. This complex, in turn, inhibits the calcium and calmodulin-dependent serine-threonine phosphatase activity of the enzyme calcineurin. Inhibition of calcineurin prevents the activation of transcription factors, such as NFAT_{p/c} and NF-κB, which are necessary for the induction of cytokine genes (IL-2, IFN-γ, IL-4, and GM-CSF) during T-cell activation.

Cyclosporin also inhibits lymphokine production by T-helper cells in vitro, and arrests the development of mature CD8 and CD4 cells in the thymus. Other in vitro properties of cyclosporin include the inhibition of IL-2 producing T-lymphocytes and cytotoxic T-lymphocytes, inhibition of IL-2 released by activated T-cells, inhibition of resting T-lymphocytes in response to alloantigen and exogenous lymphokine, inhibition of IL-1 production, and inhibition of mitogen activation of IL-2 producing T-lymphocytes.

Despite the advantageous immunosuppressive, anti-inflammatory, and anti-parasitic effects of cyclosporin, there are numerous adverse effects associated with cyclosporine A therapy. These effects include nephrotoxicity, hepatotoxicity, cataractogenesis, hirsutism, parathesis, and gingival hyperplasia, to name a few. Of these, nephrotoxicity is one of the more serious dose-related adverse effects resulting from cyclosporine administration. Immediate-release cyclosporine A drug products (e.g., Neoral® and Sandimmune®) can cause nephrotoxicities and other toxic side effects due to their rapid release into the blood stream, and the resulting high concentrations that are a consequence of rapid release. Although the precise mechanism by which cyclosporine A causes renal injury is not known, it is proposed that an increase in the levels of vasoconstrictive substances in the kidney leads to vasoconstriction of afferent glomerular arterioles. This can result in renal ischemia, a decrease in glomerular filtration rate and, over the long term, interstitial fibrosis.

Accordingly, there is a need for immunosuppressive agents with pharmacological efficacy comparable to the naturally occurring compound cyclosporine A, but reduced toxic side effects.

Since the original discovery of cyclosporin, a wide variety of naturally occurring cyclosporins have been isolated and identified. Additionally, many cyclosporins that do not occur naturally have been prepared by partial or total synthetic means, and by the application of modified cell culture techniques. Thus, the class comprising cyclosporins is substantial and includes, for example, the naturally occurring cyclosporines A through Z; various non-naturally occurring cyclosporin derivatives; artificial or synthetic cyclosporins including the dihydro- and iso-cyclosporins; derivatized cyclosporins (for example, either the 3′-O-atom of the MeBmt residue may be acylated, or a further substituent may be introduced at the sarcosyl residue at the 3-position); cyclosporins in which the MeBmt residue is present in isomeric form (e.g., in which the configuration across positions 6′ and 7′ of the MeBmt residue is cis rather than trans); and cyclosporins wherein variant amino acids are incorporated at specific positions within the peptide sequence.

Cyclosporin analogs containing modified amino acids in the 1-position are disclosed in WO 99/18120 and WO 03/033527, which are assigned to the assignee of the present application, and incorporated herein by reference in their entirety. These applications describe a cyclosporin derivative known as “ISA_{TX}247” or “ISA247” or “ISA.” This analog is structurally identical to cyclosporine A, except for modification at the amino acid-1 residue. Applicants have previously discovered that certain mixtures of cis and trans isomers of ISA247, including mixtures that are predominantly comprised of trans ISA247, exhibited a combination of enhanced potency and reduced toxicity over the naturally occurring and presently known cyclosporins. Certain alkylated, arylated, and deuterated derivatives of ISA247 have also been disclosed.

The metabolites of cyclosporine A have been studied, and in some cases, have been found to have an efficacy as potent as that of the parent drug. In addition, metabolites have been used to produce antibodies which recognize the metabolites. These antibodies can be used to monitor the amount of drug in a patient's blood (therapeutic drug monitoring or TDM). Antibodies which specifically recognize metabolites may be useful in performing these TDM tests by binding metabolites which may otherwise cause falsely high calculations of the amount of drug in the patient's blood. Accordingly, there is a need in the art for identifying and isolating metabolites of ISA247, as well as methods of preparation and use of these metabolites.

### SUMMARY OF THE INVENTION

The present invention relates to the identification and isolation of metabolites of the cyclosporine analog ISA247. The present invention also provides methods of preparation and use of metabolites of the cyclosporine analog ISA247. Such metabolites are contemplated to have a useful immunosuppressive activity, and may display a toxicity less than or equal to that of the parent compound. They can also be useful for the development of assays for therapeutic drug monitoring.

In embodiments of the present invention, metabolites of ISA247 include an isolated compound represented by the following formula:

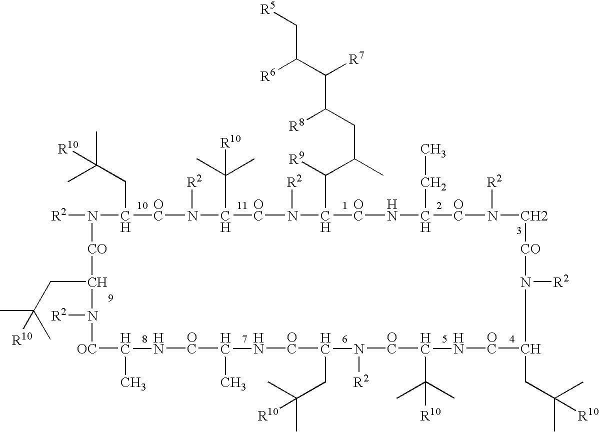

and pharmaceutically acceptable salts and solvates thereof, wherein:

each R² is independently —H or —CH₃;

each R¹⁰ is independently —H, —OH, —F, —Cl, —Br, —I, —CN, —NO₂, —OR^{a}, —C(O)R^{a}, —OC(O)R^{a}, —C(O)OR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{C}C(O)R^{a}, —NR^{C}C(O)OR^{a} or —NR^{c}C(O)N(R^{a}R^{b});

R⁵, R⁶, R⁷, R⁸ and R⁹ are independently —H, —OH, —F, —Cl, —Br, —I, —CN, —NO₂, —OR^{a}, —C(O)R^{a}, —OC(O)R^{a}, —C(O)OR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(₉)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a} or —NR^{c}C(O)N(R^{a}R^{b}); or R⁶ and R⁷ are together —O—; or R⁵ and R⁶ together, or R⁷ and R⁸ together, are independently —O—; or R⁸ and R⁹ together are —O—; or R⁵, together with the carbon to which it is bonded, is —C(═)R^{a}, —CO₂R^{a}, —CH₂N R^{a}, —CH₂OC(O)R^{a}, —CH(OR^{a})₂, —C(O)N(R^{a}R^{b}), —C(═NR^{b})R^{a}, —C(═NOR^{b})R^{a}, or —C(═NNR^{b})R^{a}; provided that one pair of R⁵ and R⁶, R⁶ and R⁷, or R⁷ and R⁸ is a carbon-carbon bond and the remainder are not all —H; and

R^{a}, R^{b} and R^{c} are each independently —H or an optionally substituted aliphatic, cycloaliphatic, benzyl, or aryl, or —N(R^{a}R^{b}) together is an optionally substituted heterocyclic group, or —CH(OR^{a})₂ together is a cyclic acetal group.

In various embodiments, the compound is represented by the following formula:

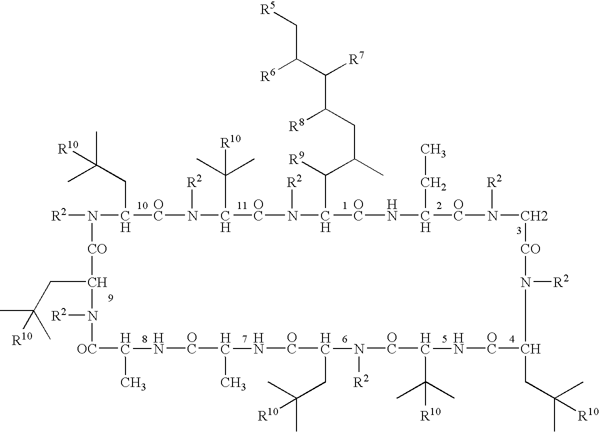

wherein:

each R² is independently —H or —CH₃;

each R¹⁰ is independently —H, —OH, —F, —Cl, —Br, —I, —OR^{a}, —OC(O)R^{a}, —OSO₂R^{a}, —OSO₃R^{a}, —OPO₂R^{a}R^{b} or —OPO₃R^{a}R^{b};

R⁵, R⁶, R⁷, R⁸ and R⁹ are independently —H, —OH, —F, —Cl, —Br, —I, —OR^{a}, —OC(O)R^{a}, —OSO₂R^{a}, —OSO₃R^{a}, —OPO₂R^{a}R^{b} or —OPO₃R^{a}R^{b}; or R⁶ and R⁷ are together —O—; or R⁵ and R⁶ together, or R⁷ and R⁸ together, are independently —O—; or R⁸ and R⁹ together are —O—; or R⁵, together with the carbon to which it is bonded, is —C(═O)R^{a}, —CO₂R^{a}, —CH₂OR^{a}, —CH₂OC(O)R^{a}, —CH(OR^{a})₂, —C(O)N(R^{a}R^{b}), —C(═NR^{b})R^{a}, —C(═NOR^{b})R^{a} or —C(═NNR^{b})R^{a}; provided that one pair of R⁵ and R⁶, R⁶ and R⁷, or R⁷ and R⁸ is a carbon-carbon bond and the remainder are not all —H; and

R^{a}, R^{b} and R^{c} are each independently —H or an optionally substituted aliphatic, cycloaliphatic, benzyl, or aryl, or —N(R^{a}R^{b}) together is an optionally substituted heterocyclic group, or —CH(OR^{a})₂ together is a cyclic acetal group.

In certain embodiments, the compound is represented by the following formula:

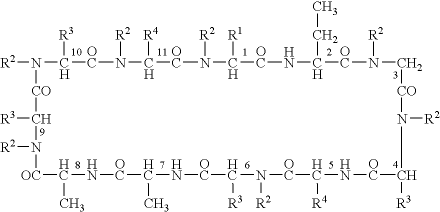

wherein:

R¹ is selected from the group consisting of

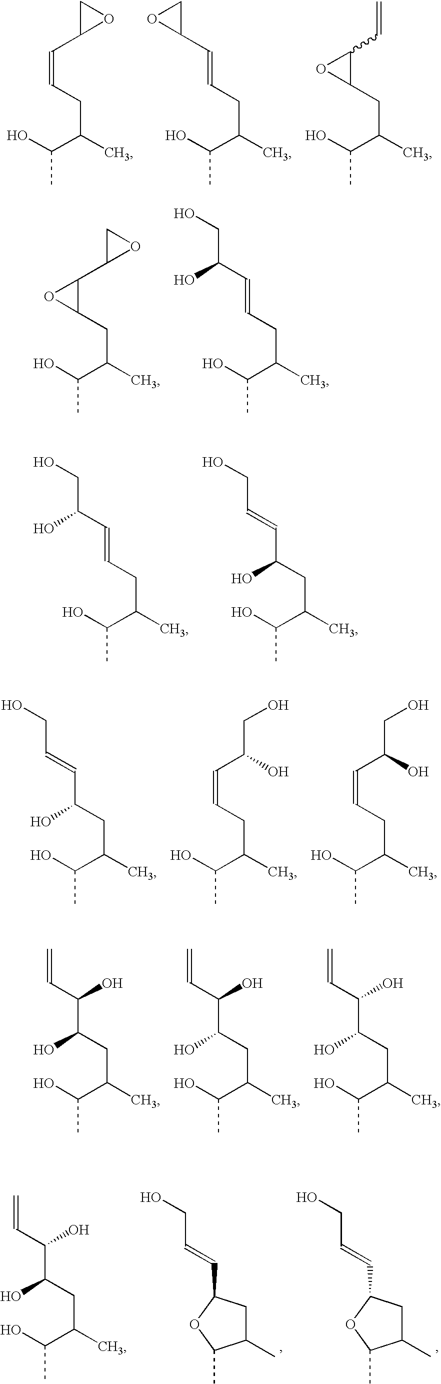

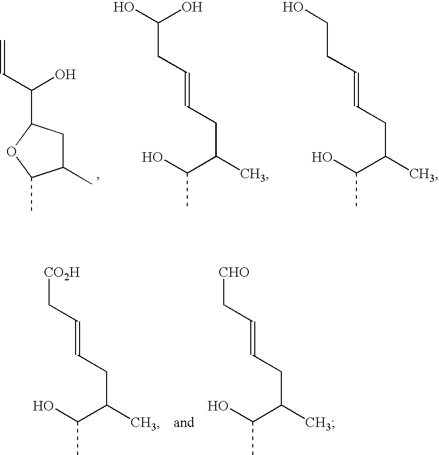

each R² is independently selected from the group consisting of —CH₃ and —H;

each R³ is independently selected from the group consisting of —CH₂CH(CH₃)₂ and —CH₂C(CH₃)₂OH; and

each R⁴ is independently selected from the group consisting of —CH(CH₃)₂ and —C(CH₃)₂OH.

Various embodiments of the invention include an isolated metabolite of cyclo {{(E)- and (Z)-(2S,3R,4R)-3-hydroxy-4-methyl-2-(methylamino)-6,8-nonadienoyl}-L-2-aminobutyryl-N-methyl-glycyl-N-methyl-L-leucyl-L-valyl-N-methyl-L-leucyl-L-alanyl-D- alanyl-N-methyl-L-leucyl-N-methyl-L-leucyl-N-methyl-L-valyl} (ISA247) and pharmaceutically acceptable salts and solvates thereof, wherein compared to ISA247, the isolated metabolite comprises at least one chemical modification selected from the group consisting of hydroxylation, N-demethylation, diol formation, epoxide formation, intramolecular cyclization, phosphorylation, sulfation, glucuronide formation and glycosylation.

In particular embodiments, the invention provides at least one chemical modification of the parent compound ISA247, wherein the chemical modification is selected from the group consisting of hydroxylation, N-demethylation, diol formation, epoxide formation, and intramolecular cyclization.

Certain embodiments of the present invention include a metabolite of cyclo {{(E)- and (Z)-(2S,3R,4R)-3-hydroxy-4-methyl-2-(methylamino)-6,8-nonadienoyl}-L-2-aminobutyryl-N-methyl-glycyl-N-methyl-L-leucyl-L-valyl-N-methyl-L-leucyl-L-alanyl-D- alanyl-N-methyl-L-leucyl-N-methyl-L-leucyl-N-methyl-L-valyl} (ISA247) comprising at least one chemical modification of the parent compound, wherein the chemical modification is selected from the group consisting of hydroxylation, N-demethylation, diol formation, epoxide formation, and intramolecular cyclization.

In various embodiments, the isolated metabolite comprises at least one chemical modification selected from the group consisting of: an epoxide at a side chain of amino acid-1; a diol at the side chain of amino acid 1; a cyclic ether in the side chain of amino acid-1; a demethylated amino nitrogen at amino acid-1, 3, 4, 6, 9, 10, or 11; an —OH at the γ carbon of the side chain of amino acid 4, 6, 9, or 10; and an —OH at the β carbon of the side chain of amino acid 5 or 11. In various embodiments, the isolated metabolite comprises two or more of the preceding chemical modifications. In specific embodiments, the isolated metabolite can be selected from the group consisting of IM1-e-1, IM1-e-2, IM1-e-3, IM1-d-1, IM1-d-2, IM1-d-3, IM1-d-4, IM1-c-1 and IM1-c-2.

In certain embodiments, compared to ISA247 the isolated metabolite comprises chemical modifications selected from the group consisting of: at least two —OH groups; at least two demethylated amino acid nitrogens; at least one —OH group and at least one demethylated amino acid nitrogen; at least one diol group and at least one —OH group; at least one diol group and at least one demethylated amino acid nitrogen; at least one cyclic ether and at least one —OH group; at least one cyclic ether and at least one demethylated amino acid nitrogen; at least one —OH group and a phosphate, sulfate, glucuronide or glycosylation residue; and at least one diol and a phosphate, sulfate, glucuronide or glycosylation residue. Embodiments include metabolites at the amino acid-1 of ISA247, including epoxides, diols and cyclizations. Additional embodiments include metabolites where the ISA247 compound has been hydroxylated at: 1) at least one methyl leucine amino acid, for example amino acids 4, 6, 9 or 10; 2) at valine residue 5; 3) or methyl valine residue 11. Further embodiments include metabolites where at least one methylated nitrogen of an amide linkage of ISA247 has been demethylated. Still further embodiments include metabolites where at least one nitrogen of the amide linkage of the amino acids 1, 3, 4, 6, 9, 10 and 11 of ISA247 has been demethylated. Exemplary metabolites of ISA247 include IM1-d-1, IM1-d-2, IM1-d-3, IM1-d-4, IM9, IM1-c-1, IM1-c-2, IM4n, IM6, IM46, IM69, IM49, IM1-e-1, IM1-e-2, and IM1-e-3.

Additional embodiments include metabolites which have a diol or a cyclization at the amino acid-1 of ISA247 combined with at least one hydroxylation or at least one N-demethylation at another amino acid residue. Additionally, embodiments include metabolites which have a diol or a cyclization at the amino acid-1 of ISA247 combined with at least one hydroxylation and at least one N-demethylation at another amino acid residue.

In various embodiments, the metabolites of ISA247 may be isolated from body fluids after administration of the drug, or may be produced either semi-synthetically (i.e., from either the microsomal homogenates of animal liver cells, or cultures of microorganisms), or entirely synthetically, such as by chemically modifying the parent compound using reactions known in the art of organic synthesis.

Thus, some embodiments of the present invention provide a method of preparing a metabolite of ISA247 in vitro, comprising the steps of a) homogenizing mammalian cells (e.g., mammalian liver cells) to form a homogenate (e.g., to rupture their plasma membranes and to release the contents of the liver cells); b) centrifuging the homogenate to yield a microsomal pellet containing at least one drug-metabolizing enzyme, for example cytochrome P450; and c) preparing a reaction mixture containing ISA247, the microsomal pellet, an energy source, and an electron donating species under conditions which result in production of at least one metabolite of ISA247. In further embodiments, the method may utilize mammalian liver cells selected from the group consisting of primate, rat, dog and rabbit, or in some embodiments from a dog or rabbit. The electron donating species can be, for example, NADH or NADPH. The energy source can be, for example selected from the group consisting of glucose-6-phosphate and isocitrate. In certain embodiments, the reaction mixture further includes an enzyme selected from the group consisting of glucose-6-phosphate dehydrogenase and isocitrate dehydrogenase.

In additional embodiments, the present invention provides a method for separating metabolites of ISA247, using high performance liquid chromatography. In further embodiments, the chromatography columns may be n-octadecyl, n-octyl, n-butyl, diphenyl, and cyanopropyl columns, and may have lengths ranging from about 150 to 250 mm, diameters ranging from about 0.1 to 4.6 mm, and flow rates of about 500 to 2,000 μL/min. Still further, an embodiment of the present invention provides that the metabolites may be identified by mass spectrometry.

Also, an embodiment of the present invention provides a method of producing a hydroxylated metabolite of ISA247, the method comprising the steps of: a) protecting the β-alcohol of the 1-amino acid residue of ISA247 to form a protected-ISA247 compound; b) halogenating the protected-ISA247 compound with a halogenating agent at the γ-carbon of the side chains of at least one of the 4, 6, or 9-amino acid residues, thereby forming a halogenated product; c) heating the halogenated product of step b) in the presence of an acetate reagent to form an acetate-containing product having an acetate moiety; and d) performing a transesterification to exchange the acetate moiety of the acetate-containing product of step c) with an alcohol moiety, thereby forming the hydroxylated metabolite of ISA247. In particular embodiments, the halogenating agent can be, for example, N-bromosuccinimide (NBS) and the acetate reagent can be, for example, tetrabutylammonium acetate.

Various embodiments include an isolated hydroxylated metabolite of ISA247 produced by the preceding method. Exemplary isolated hydroxylated metabolites can be selected from the group consisting of IM9, IM4, IM6, IM46, IM69 and IM49.

Embodiments of the present invention provide a method of producing an epoxide metabolite of ISA247 in vitro, comprising the step of oxidizing an alkene moiety of the side chain of the 1-amino acid residue of isolated ISA247 with an oxidizing agent (e.g., using the Prilezhaev reaction), thereby forming the epoxide metabolite of ISA247. The oxidizing agent can be, for example, m-chloroperbenzoic acid (MCPBA), peracetic acid, trifluoroperacetic acid, perbenzoic acid, 3,5-dinitroperbenzoic acid, hydrogen peroxide, alkyl peroxide, oxygen, or the like.

Various embodiments include an isolated epoxide metabolite of ISA247 prepared by the preceding method. Exemplary isolated isolated epoxide metabolites include IM1-e-1, IM1-e-2 and IM1-e-3.

An additional embodiment of the present invention provides a method of producing a diol containing metabolite of ISA247, the method comprising the steps of: a) protecting the β-OH group; b) treating an alkene moiety of the side chain of the amino acid-1 residue of ISA247 with an oxidizing agent, such that the alkene moiety is converted to a mono-epoxide; c) forming the diol containing metabolite of ISA247 from the epoxide; and d) deprotecting the β-OH group using a base.

Additional embodiments of the present invention include a method of producing a diol containing metabolite of ISA247, the method comprising the steps of: a) treating an alkene moiety of the side chain of the 1-amino acid residue of ISA247 with an oxidizing agent to form a epoxide metabolite of ISA247 (e.g., as a Prilezhaev reaction); and b) forming the isolated diol metabolite of ISA247 from the isolated epoxide metabolite of ISA247. Typical examples of the oxidizing agent include m-chloroperbenzoic acid (MCPBA), peracetic acid, triflouroperacetic acid, perbenzoic acid, 3,5-dinitroperbenzoic acid, hydrogen peroxide, alkyl peroxide, and oxygen. In another embodiment, step b) that forms the diol containing metabolite of ISA247 from the epoxide comprises subjecting the epoxide of step a) to a nucleophilic attack using water(e.g., hydrolysis). The nucleophilic attack by water may be catalyzed by an agent selected from the group consisting of an acid and a base, for example, perchloric acid, alkaline water, Nafion-H, formic acid, or the like. In particular embodiments, the hydrolysis in step b) is selected from hydrolysis catalyzed by perchloric acid or Nafion-H; alkaline hydrolysis in dimethyl sulfoxide; and hydrolysis catalyzed by microsomal epoxide hydrolase.

Various embodiments include an isolated diol metabolite of ISA247 prepared by the preceding method. Exemplary isolated diol metabolites include IM1-d-1, IM1-d-2, IM1-d-3 and IM1-d-4.

In a still further embodiment, the present invention provides a method of producing a diol-containing metabolite of ISA247, comprising the step of forming the diol metabolite directly from ISA247 using osmium tetroxide and alkaline potassium permanganate or hydrogen peroxide, or t-butyl hydroperoxide or hydrogen peroxide/formic acid, or monopersuccinic acid. In typical embodiments, the ISA247 is reacted with a reagent selected from the group consisting of osmium tetroxide, alkaline potassium permanganate, hydrogen peroxide, monopersuccinic acid and t-butyl hydroperoxide, thereby forming the diol metabolite of ISA247. Typically, the ISA247 can be reacted with a catalytic amount of osmium tetroxide. In some embodiments, ISA247 can be reacted with a reagent selected from the group consisting of hydrogen peroxide/formic acid and monopersuccinic acid

In some embodiments, a method of producing a diol metabolite of ISA247 includes the steps of a) treating ISA247 with a reagent selected from the group consisting of iodine/silver benzoate and silver acetate to form a diester of ISA247; and b) hydrolyzing the diester of ISA247, thereby forming the diol metabolite of ISA247.

Various embodiments include an isolated diol metabolite of ISA247 prepared by the preceding method. Exemplary isolated diol metabolites include IM1-d-1 and IM1-d-2.

In some embodiments, a method of producing a diol metabolite of ISA247 includes the steps of a) reacting ISA247 with a reagent selected from the group consisting of lead tetraacetate and thallium acetate to form a diol bisacetate of ISA247; and b) hydrolyzing the diol bisacetate of ISA247, thereby forming the diol metabolite of ISA247.

Various embodiments include an isolated diol metabolite of ISA²⁴⁷ prepared by the preceding method. Exemplary isolated diol metabolites include IM1-d-1 and IM1-d-2.

In an additional embodiment, the metabolite diols are prepared from vinyl epoxides which are accessible by organometallic moieties such as haloallylborations. Such vinyl epoxides are also available by the Sharpless dihydroxylation protocol.

Particular embodiments of the invention include pharmaceutical compositions comprising a pharmaceutically acceptable carrier and any of the isolated compounds or metabolites of the invention.

### BRIEF DESCRIPTION OF THE DRAWINGS

FIG. 1 is an illustration of the structure of CsA.

FIG. 2 is an illustration of the structure of ISA247.

FIG. 3 is an illustration of the E (trans) isomer of ISA247.

FIG. 4 is an illustration of the Z (cis) isomer of ISA247.

FIG. 5 is a table showing known CsA metabolites.

FIG. 6 is an HPLC-MRM scan of metabolites present in human whole blood after administration of ISA247.

FIG. 7 is an HPLC-MRM scan of a mixed standard of isolated ISA247 metabolites.

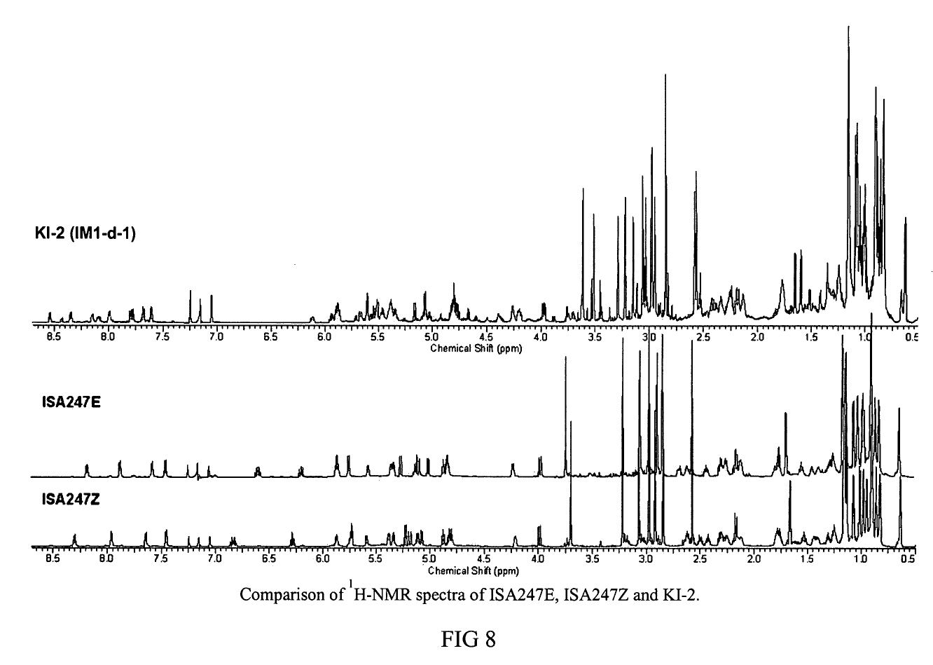

FIG. 8 shows ¹H-NMR spectra for E-ISA247, Z-ISA247 and IM 1-d-1, from sample KI-2.

FIG. 9 is 2D TOCSY spectrum of IM1-d-1, from sample KI-2.

FIG. 10 shows the structure(s) of IM1-d-1.

FIG. 11 shows ¹H-NMR spectra for E-ISA247 and IM1-d-2, from sample KI-3A.

FIG. 12 shows 2D COSY and TOCSY spectra of IM1-d-2, from sample KI-3A.

FIG. 13 shows expanded 2D COSY spectrum of IM1-d-2, from sample KI-3A, between 3.8 and 6.2 ppm.

FIG. 14 shows expanded ¹H-NMR spectrum of IM1-d-2, from sample KI-3A, between 3.8 and 6.2 ppm.

FIG. 15 shows expanded ¹H-NMR spectrum of the double bond protons of IM1-d-2 (sample KI-3A), indicating the splitting patterns of the signal.

FIG. 16 shows expanded 2D COSY spectrum of IM1-d-2 (sample KI-3A) showing the correlation of the aa-1 side chain protons.

FIG. 17 illustrates the structure of R and S epimers, at the ε position of the aa-1 side chain, of IM1-d-2.

FIG. 18 shows the amino acid-1 structure and its proton chemical shifts of IM1-d-2.

FIG. 19 shows ¹H-NMR spectra for IM1-d-1, IM1-d-3, E-ISA247 and Z-ISA247.

FIG. 20 shows 2D TOCSY spectrum of IM1-d-3 (sample KI-3).

FIG. 21 shows the amide proton correlations to the corresponding a protons in expanded TOCSY spectra of IM1-d-3 (sample KI-3).

FIG. 22 also shows the amide proton correlations to the corresponding side chain methyl protons in expanded TOCSY spectra of IM1-d-3 (sample KI-3).

FIG. 23 shows expanded ¹H NMR spectrum of IM1-d-3 (sample KI-3) between 3.5 and 6.1 ppm and some TOCSY correlations.

FIG. 24 shows expanded 2D TOCSY spectrum of IM1-d-3 (sample KI-3) between 3.4 and 6.3 ppm with cross peak correlations.

FIG. 25 shows expanded ¹H-NMR spectrum of EM1-d-3 (sample KI-3) with analysis of the signal at δ 5.62 ppm.

FIG. 26 shows the structures of the R and S isomers, at the η position of the aa-1 side chain, of IM1-d-3.

FIG. 27 shows ¹H-NMR spectra of IM1-d-4 (sample KI-8A) and E-ISA247.

FIG. 28A shows 2D COSY spectrum, and FIG. 28B shows 2D TOCSY spectrum of IM1-d-4 (sample KI-8A).

FIG. 29 shows expanded 2D COSY spectrum of IM1-d-4 (sample KI-8A) between 3.5 and 6.2 ppm with correlations.

FIG. 30 is an expanded ¹H-NMR spectrum of IM1-d-4 (sample KI-8A) between 3.7 and 6.2 ppm with COSY correlations and some proton assignments.

FIG. 31 are first order analyses of ¹H-NMR signals at 6-6.00, 5.44 and 5.15 ppm for IM1-d-4 (sample KI-8A).

FIG. 32 illustrates the structure of IM1-d-4.

FIG. 33 compares ¹H-NMR spectra for E-ISA247, Z-ISA247 and IM1-c-1 (sample KI-5).

FIG. 34 shows an expanded ¹H-NMR spectrum for IM1-c-1 (sample KI-5) at the α proton region.

FIG. 35 shows analysis of signals at δ˜5.75 ppm of IM1-c-1 (sample KI-5), as shown in FIG. 34.

FIG. 36 shows expanded 2D TOCSY spectrum of IM1-c-1 (sample KI-5) at the α proton region.

FIG. 37 is a partially expanded DQF-COSY spectrum of IM1-c-1 (sample KI-5).

FIG. 38 illustrates the structure of IM1-c-1.

FIG. 39 is 2D ROESY spectrum of IM1-c-1 (sample KI-5).

FIG. 40 is an illustration of a structure for the amino acid-1 side chain of IM1-c-1 showing ROE correlations.

FIG. 41 is an exemplary reaction scheme illustrating the formation of amino acid-1 metabolites of ISA247.

FIG. 42A is an exemplary reaction scheme illustrating the formation of amino acid-1 metabolites of ISA247 from trans-ISA247.

FIG. 42B is an exemplary reaction scheme illustrating the formation of amino acid-1 metabolites of ISA247 from cis-ISA247.

FIG. 43 is an exemplary reaction scheme illustrating the formation of 1M1-d-1 from trans-ISA247 and IM1-d-3 from cis ISA247.

FIG. 44A is a comparison of the ¹H-NMR spectra of KI-7C, E-ISA247 and Z-ISA247.

FIG. 44B illustrates the structure of IM9.

FIG. 45 is a comparison of the ¹H-NMR spectra of IM4 (sample KI-6), ISA247 E and Z-ISA247.

FIG. 46 is a comparison of the expanded ¹H-NMR spectra of IM4 (sample KI-6), ISA247 E and Z-ISA247 between 0.5 and 1.5 ppm.

FIG. 47 is an expanded ¹H-NMR showing new methyl signals of IM4.

FIG. 48 is 2D TOCSY spectrum of IM4 (sample KI-6).

FIG. 49 is a scheme showing a transformation of the amino acid-4 γ position to form IM4.

FIG. 50 illustrates the structure of IM4.

FIG. 51 is a comparison of the ¹H-NMR spectra of IM4n (sample KI-1), ISA247 E and Z-ISA247.

FIG. 52 is an expanded 2D TOCSY spectrum of IM4n (sample KI-1).

FIG. 53 illustrates the structure of IM4n.

FIG. 54 illustrates chemical reaction schemes using Sharpless methods.

FIG. 55 illustrates chemical synthetic methods for directing the synthesis of specific syn or anti diols.

FIG. 56 illustrates chemical synthetic methods using chloroallylboration.

FIG. 57A is an exemplary reaction scheme illustrating the formation of IM-1, IM-1-acetal, IM1-aldehyde, and 1M1-carboxylic acid from E-ISA247.

FIG. 58A is a graph showing percent calcineurin inhibition versus concentration of metabolite in ng/mL for ISA247 metabolites IM1-diol-1, IM9, IM4n, IM1c, and IM1.

FIG. 58B is a graph showing percent calcineurin inhibition versus concentration in ng/mL for trans-ISA247, cis ISA247, and CsA.

FIG. 59 is a bar graph showing a typical metabolic diversity profile of ATCC 11635 in ISA247 metabolite production. The conversion shown is a percentage compared to a 1 mg/mL Cyclosporine A standard.

### DETAILED DESCRIPTION OF THE INVENTION

The present invention identifies metabolites of the cyclosporine analog ISA247. The present invention also provides methods of preparation of metabolites of the cyclosporine analog ISA247. Such metabolites have immunosuppressive activity. They are also useful for the development of assays for therapeutic drug monitoring, including the production of antibodies.

Cyclosporine A is a neutral, highly lipophilic cyclic undecapeptide produced in submerged cultures of the species Tolypocladium inflatum Gams. It has the clinical code OL 27-400, and is the active ingredient of the immunosuppressive formulation bearing the trademark Sandimmune®.

Cyclosporine A has been recently renamed “cyclosporine” (according to Carruthers, 1983). In the present application, the abbreviation “C_{S}A” will be used to denote the particular compound cyclosporine A, and the term “cyclosporin” is intended to refer to the general class of immunosuppressive agents that comprise cyclic peptides having immunsuppressive activity, including cyclosporin analogs. Therefore, the term “cyclosporin” generally refers to any of the cyclosporines A through Z, including modifications and analogs. In particular, the term cyclosporin includes the compounds C_{S}A and ISA247.

Structure and Nomenclature of CsA and its Metabolites

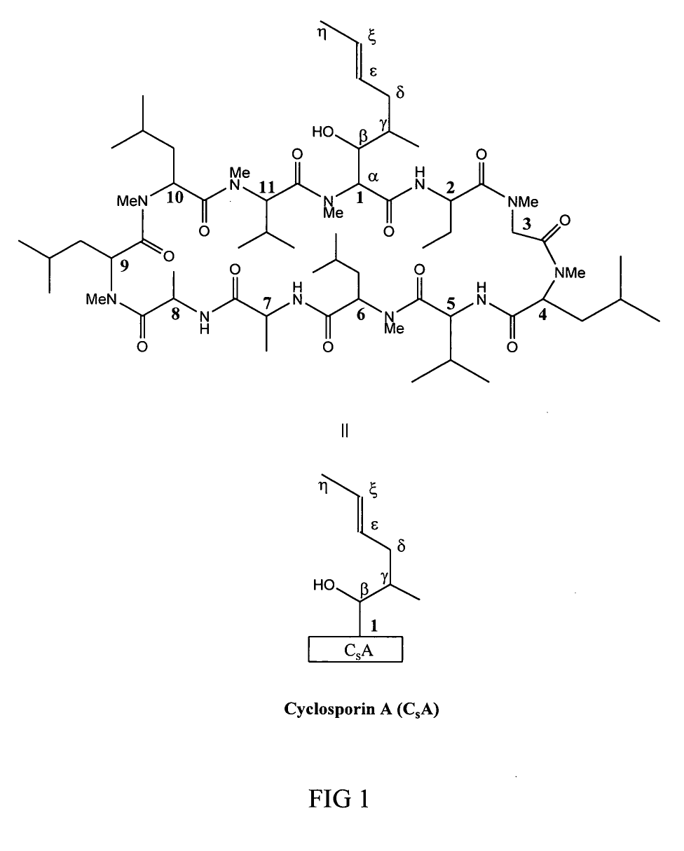

The structure of cyclosporine A is shown in FIG. 1. As described by N. R. Hartman and I. Jardine in “Mass Spectrometric Analysis of Cyclosporine Metabolites,” Biomed. Environ. Mass Spectrom. Vol. 13, pp. 361-372 (1986), cyclosporine A is a cyclic undecapeptide consisting almost entirely of hydrophobic amino acids. Many of these amino acids are not normally found in proteins. FIG. 1 identifies the 11 amino acid residues that comprise the cyclic peptide ring of this molecule. The C_{S}A molecule contains a sarcosine residue (Sar or methylated glycine residue MeGly), one each of a D- and L-alanine (Ala) residue, an cc-amino butyric acid residue (Abu), a valine (Val) residue, an N-methyl valine (MeVal) residue, four N-methyl leucine (MeLeu) residues, and an alkene-containing 9-carbon, β-hydroxylated amino acid unique to the cyclosporins called (4R)-4-[(E)-2-butenyl]-4,N-dimethyl-L-threonine (MeBmt).

As used herein, the amino acid residues are sequentially numbered 1-11 beginning with (4R)-4-[(E)-2-butenyl]-4,N-dimethyl-L-threonine (MeBmt) and ending at the adjacent MeVal (in FIG. 1, numbered clockwise starting at MeBmt).

Each of the amino acids of C_{S}A has an S-configuration (the L-isomer of the amino acid) with the exception of the alanine residue at position 8, which takes the R-configuration (the D-isomer of alanine). Seven of the amino acids are methylated at their amine nitrogen atoms; these are residues 1, 3, 4, 6, 9, 10, and 11. At the time of the discovery of the structure of cyclosporine A, the ten amino acids 2-11 were known, but the β-hydroxylated amino acid MeBmt was not.

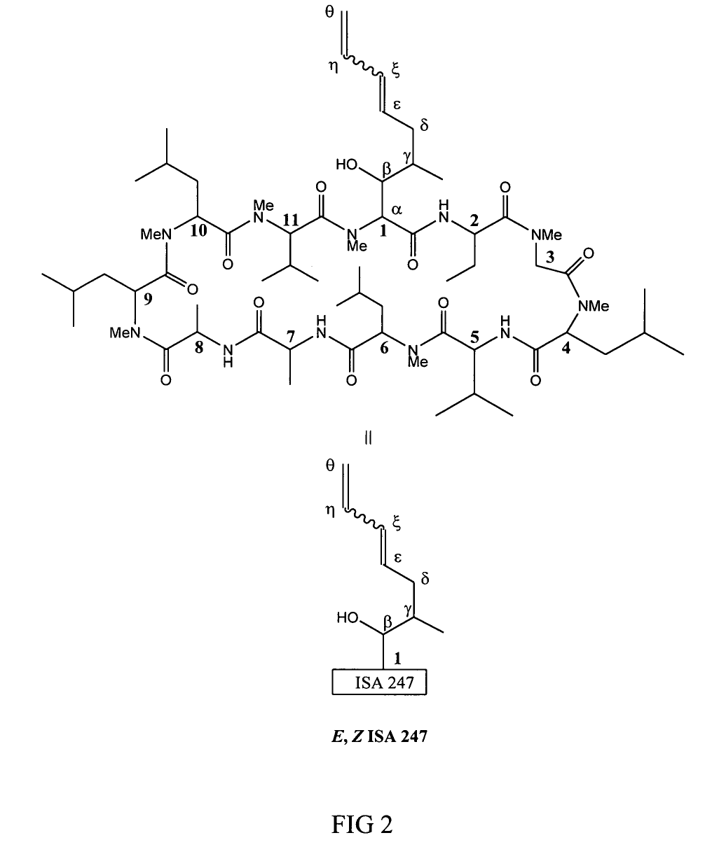

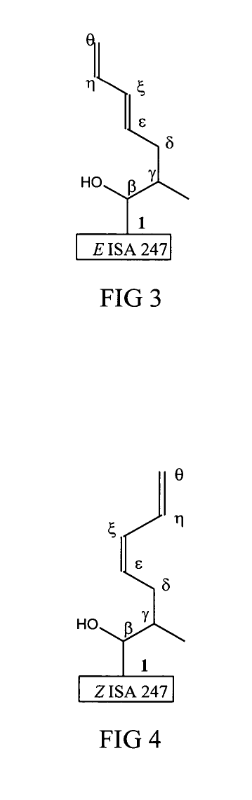

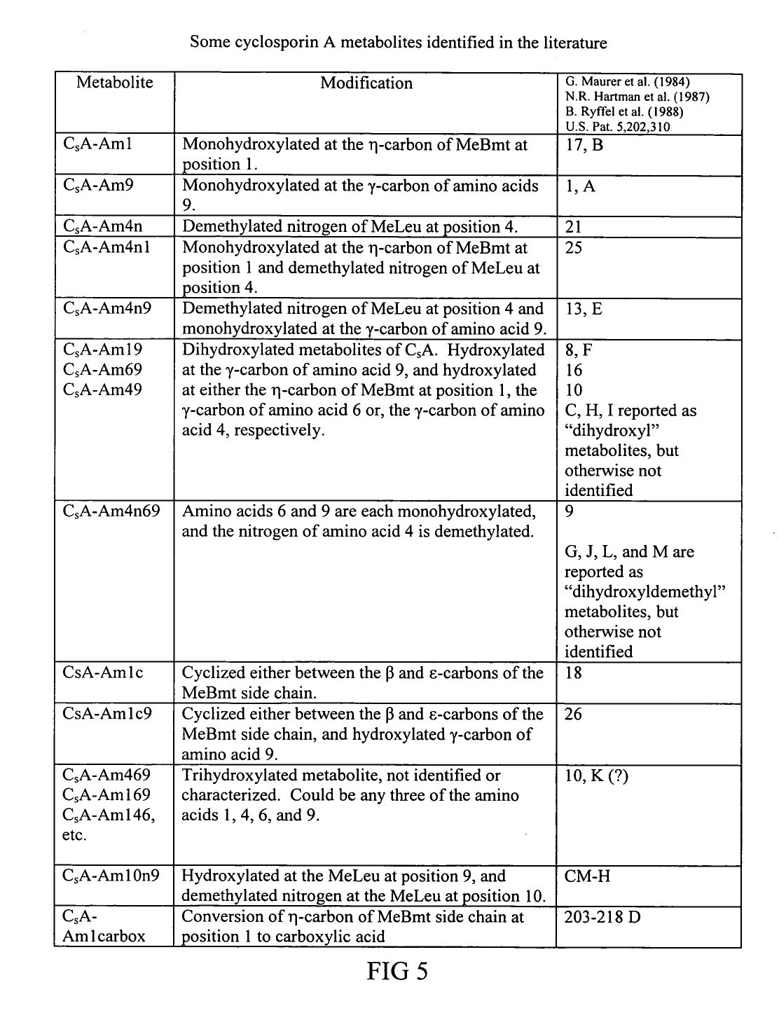

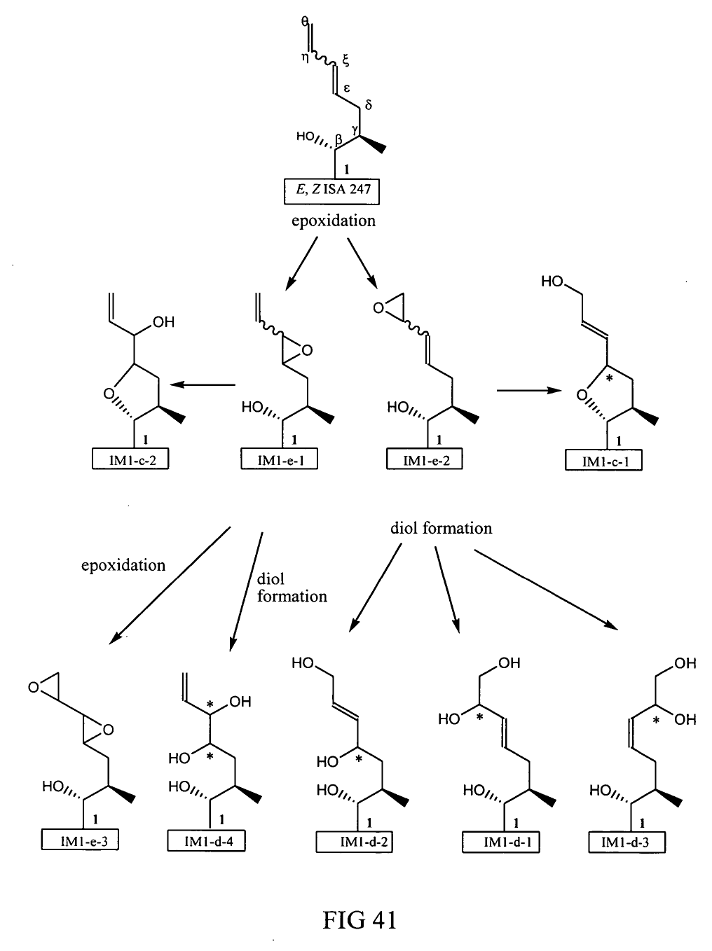

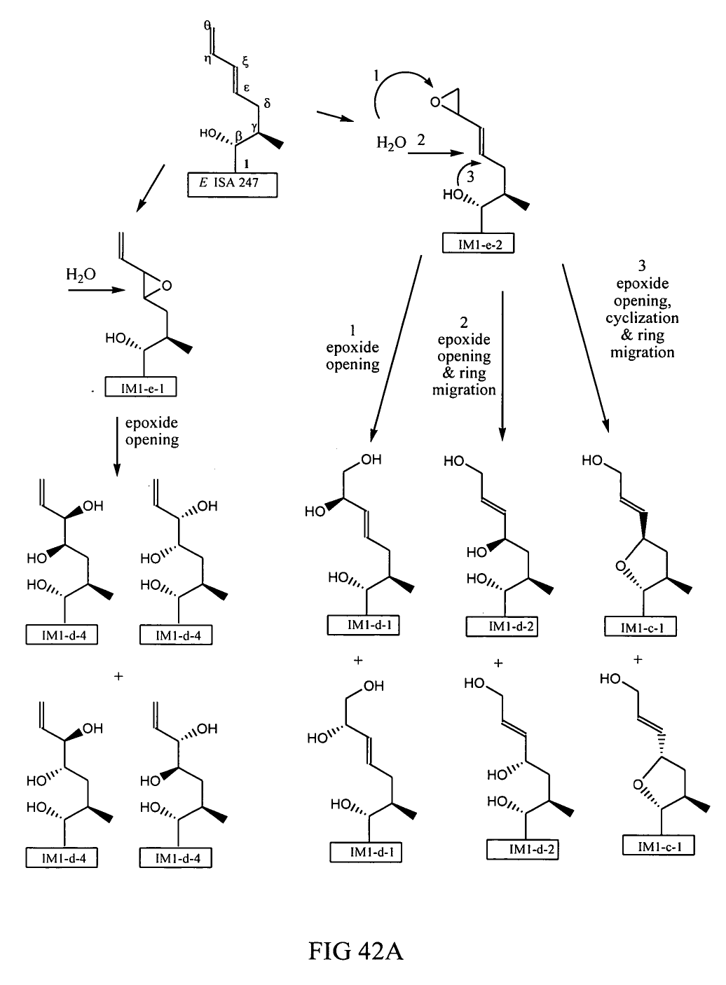

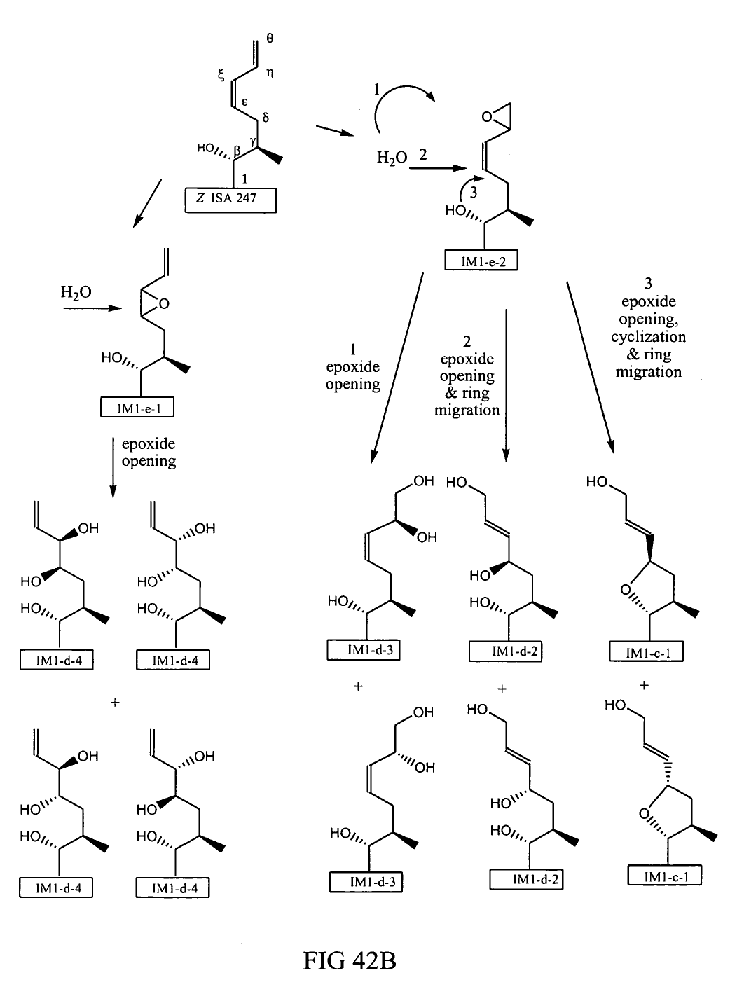

The structure of ISA247 (or ISA_{TX}247 or ISA) is shown in FIG. 2. As used herein, the amino acid residue positions in ISA247 and the disclosed metabolites are numbered as in CsA in FIG. 1. By comparison with FIG. 1, it can be seen that CsA and ISA247 are identical but for the side chain at amino acid residue 1. The common structure of these cyclosporins can be represented by the box shown in FIGS. 3 and 4, which illustrate the E (trans) and Z (cis) isomeric forms of ISA247, where the numeral 1 indicates the amino acid residue 1 to which the displayed side chain is bonded. The structure of amino acid-1 is illustrated similarly in FIGS. 41 and 42, and in Table 5.

When the structure of the side chain of an amino acid is explicitly drawn in the present disclosure, the carbons of that side chain may be labeled with Greek letters as is conventionally known in the art. For example, the carbon adjacent to the carbonyl group of an amino acid is conventionally labeled the α-carbon, with progressive letters in the Greek alphabet used to identify carbon atoms proceeding away from the peptide ring. An example of this nomenclature is shown in FIGS. 1 and 2. For example, in the case of C_{S}A, the β-carbon of the MeBmt side chain is bonded to a hydroxyl group, and there is a double bond between the ε and ζ-carbons of the side chain.

Further information about the structure of C_{S}A has been provided by P. A. Keown in a chapter entitled “Molecular and Clinical Therapeutics of Cyclosporine in Transplantation” in Immunosuppression in Transplantation (Blackwell Science, Malden Mass., 1999), pp. 1-12. According to Keown, solid state x-ray diffraction and nuclear magnetic resonance studies in nonaqueous solution show that the C_{S}A molecule is characterized by two structural motifs. Residues MeBmt (position 1) to MeLeu (position 6) comprise an antiparallel P-sheet stabilized by hydrogen bonding, while the residues Ala (position 7) to MeVal (position 11) form a loop in which the peptide bond between residues 9 and 10 is in a cis configuration. Immunosuppressive cyclosporins possess two regulatory domains. Residues 1, 2, 9, 10, and 11 represent the receptor binding domain, while the residues 4 to 8 function as the effector domain. According to R. M. Wenger in an article entitled “Synthesis of Cyclosporine and Analogs: Structural Requirements for Immunosuppressive Activity,” Angew Chem Int. Ed. Engl. Vol. 24, No. 2, pp. 77-138 (1985), the essential amino acids for immunosuppression are MeBmt, Abu, Sar, and MeVal in positions 1, 2, 3, and 11.

The cyclic undecapeptide structure of CsA is preserved in all elucidated metabolites (Copeland, 1990). The reactions involved in the biotransformation of these cyclic peptides are, for the most part, hydroxylations, epoxide formation, N-demethylations, and intramolecular cyclizations. The term “hydroxylation” refers to a monohydroxylation, although multiple hydroxylations can occur at different sites in the same molecule. A dihydroxylation may also occur by oxidation of an alkene to an epoxide, with subsequent diol formation. N-Demethylations occur at a methylated nitrogen of an amide bond linking adjacent amino acid residues in the cyclic peptide ring. Metabolites of CsA include aldehyde and carboxylic acid derivatives. The formation of carboxylic acid metabolites of CsA may lead to toxicity. Combinations of the above described reactions may occur as well.

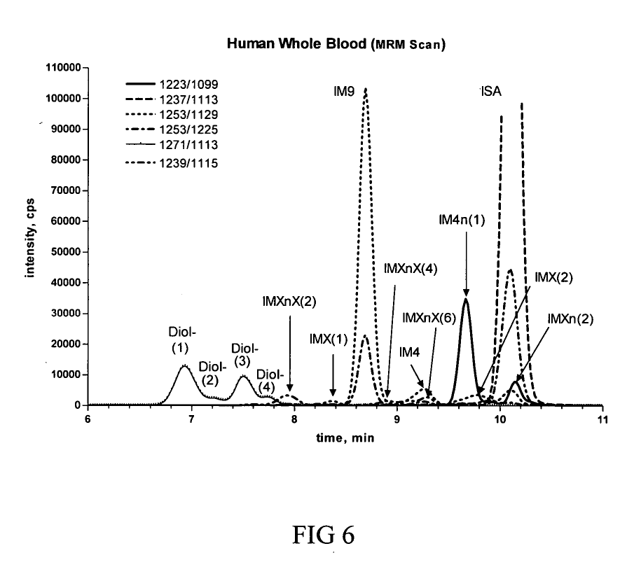

A number of different investigators have described C_{S}A metabolites, though not always using the same nomenclature. To clarify this situation, a table of known metabolites of C_{S}A has been constructed in FIG. 5. Referring to FIG. 5, the metabolites of cyclosporine A are identified by the prefix “C_{S}A-Am,” where the “C_{S}A” portion of the designation indicates the compound is a metabolite of cyclosporine A, and the “Am” part of the symbol indicates the amino acid position at which the metabolic modification takes place. The symbols that follow specify the nature of the metabolic reaction. Use of this nomenclature will become clearer with reference to the following discussion of ISA247 metabolites.

Structure and Nomenclature of ISA247 and its Metabolites

The inventors have previously disclosed a cyclosporine A analog referred to as “ISA,” “ISA247” or “ISA_{TX}247” (See U.S. Pat. Nos. 6,605,593 and 6,613,739). As noted above, this analog is structurally similar to cyclosporine A, except for modification at the amino acid-1 residue. The inventors have discovered that certain mixtures of the cis-isomer (also known as the Z-isomer) and the trans-isomer (also known as the E-isomer) of ISA247 exhibited a combination of enhanced potency and reduced toxicity relative to the naturally occurring and presently known cyclosporins. Further, the inventors have discovered that mixtures of the cis-isomer and the trans-isomer that are comprised of predominantly trans-isomer have reduced toxicity and increased potency relative to the naturally occurring and presently known cyclosporins.

The chemical name of ISA247 is cyclo{{(E)- and (Z)-(2S,3R,4R)-3-hydroxy-4-methyl-2-(methylamino)-6,8-nonadienoyl}-L-2-aminobutyryl-N-methyl-glycyl-N-methyl-L-leucyl-L-valyl-N-methyl-L-leucyl-L-alanyl-D-alanyl-N-methyl-L-leucyl-N-methyl-L- leucyl-N-methyl-L-valyl}. Its empirical formula is C₆₃H₁₁₁N₁₁O₁₂, and it has a molecular weight of about 1214.85. The terms “ISA,” “ISA247” and “ISA_{TX}247” are trade designations given to this pharmacologically active compound.

The structure of ISA247 has been verified primarily through nuclear magnetic resonance (NMR) spectroscopy. Both the ¹H and ¹³C spectra were assigned using a series of one and two dimensional NMR experiments, and by comparing ISA247 peaks to the known NMR assignments for cyclosporine A. The absolute assignment of the (E) and (Z)-isomers of ISA247 was confirmed by Nuclear Overhauser Effect (NOE) experiments. Additional supporting evidence was provided by mass spectral analysis, which confirmed the molecular weight, and by an infrared spectrum, which was found to be very similar to cyclosporine A. The latter result was expected, given the close structural similarity between the two compounds. However, ISA247 contains a conjugated diene in the side chain of its 1-amino acid residue that is not present in CSA. Like C_{S}A, ISA247 has a carbon-carbon double bond between the ε- and ζ-carbons of its amino acid-1 side chain, but unlike C_{S}A, there is an additional carbon-carbon double bond in ISA247 between the η and θ carbons.

Because of the similarity between the structures of CsA and ISA247, the nomenclature for the disclosed metabolites of ISA247 is based on the naming scheme developed for CsA metabolites. Metabolites identified herein will follow a similar pattern, except that ISA247 metabolites are preceded with the prefix “I” for “ISA” instead of “A” for cyclosporine “A.” And, while the letters “AM” are used in identifying the modified amino acid for CsA metabolites, ISA metabolites will use “IM.” For example, the CsA metabolite which is a monohydroxylation at the γ-carbon of amino acid-9 is identified as CsA-Am9 or AM9. The ISA247 metabolite which is a monohydroxylation at the γ-carbon of amino acid-9 is identified as IM9. The CsA metabolite which is a demethylation of the nitrogen of MeLeu at position 4 is identified as CsA-Am4n or AM4n. The ISA247 metabolite which is a demethylation of the nitrogen of MeLeu at position 4 is identified as IM4n (ISA Metabolite at amino acid-4, n-demethylation).

Because ISA247 has a 1,3 diene at the amino acid-1, several diol metabolites can be formed from ISA247 that cannot be formed from CsA. The nomenclature for these diol metabolites will mirror the standards presented above. For example, the first diol metabolite for which a structure was elucidated is IM1-d-1 (ISA Metabolite at the amino acid-1 -diol-1^{st} structure examined).

Like CsA, ISA247 can be metabolized in vivo to form metabolites. These metabolites are carried through the blood stream and can be excreted through urine and/or bile. Therefore, ISA247 metabolites can be isolated from body fluids, including whole blood, bile and urine of animals after administration of the drug. ISA247 metabolites can also be produced by microorganisms through biotransformation. In addition, ISA247 metabolites can be prepared using mammalian microsome systems. ISA247 metabolites can also be synthesized chemically. Metabolites can be isolated and characterized by chromatographic techniques coupled with mass spectrometry and by Nuclear Magnetic Resonance (NMR) techniques.

In additional embodiments, the present invention provides antibodies that specifically recognize the metabolites of this invention. Particularly contemplated are antibodies that recognize a given metabolite but do not cross-react with cyclosporine, ISA247 or other metabolites. The antibodies may be polyclonal, monoclonal, multispecific, human, humanized, primatized, chimeric antibodies, single chain antibodies, epitope-binding fragments (e.g., Fab, Fab′ and F(ab′)2), and the like. The metabolites of this invention can be used to prepare and/or screen antibodies. Furthermore, if a polyclonal antibody mixture (such as an antiserum) cross-reacts with cyclosporine, ISA247, or an undesired metabolite, the mixture can be treated by immunoselection or immunoabsorption to remove the cross-reacting antibodies. For example, in immunoselection, the mixture can be passed through a column to which the metabolite of interest is immobilized. The antibodies that bind to the column can then be eluted and collected. Conversely, in immunoabsorption, the compounds that the mixture cross-reacts with can be immobilized and used to absorb undesired antibodies. Methods of preparing antibodies are known in the art (see, e.g., Harlow and Lane, “Antibodies. A Laboratory Manual”, Cold Spring Harbor Laboratory, New York, 1988). The antibodies are useful, for example, in therapeutic drug monitoring.

Definitions

As used herein, “metabolite” means a derivative of ISA247 produced by metabolism of ISA247 in a mammal, which may be further chemically modified. The isolated metabolites of ISA247 disclosed herein may be prepared by administration of ISA247 to a mammal, followed by isolation; by chemical modification as described herein of ISA247, CsA, or another cyclosporine derivative; by in vitro reaction of ISA247, CsA, or another cyclosporine derivative with enzyme; by microbial conversion of ISA247, CsA or another cyclosporine derivative or by a combination of two or more of these steps in any order. For example, ISA247 may be administered to a mammal, and a metabolite of ISA247 isolated from the mammal may be chemically modified to form a further metabolite; or ISA247 may be reacted in vitro with a microsomal preparation as described herein to form a metabolite which can be chemically modified to form a further metabolite.

As used herein, “chemically modified” means that a compound has at least one chemical structural difference compared to a reference structure. A chemical modification can be produced by any synthetic, enzymatic, or metabolic process as described herein.

As used herein, suitable protecting groups, e.g., for protecting the β-alcohol of the 1-amino acid residue of ISA247 to form a protected-ISA247 compound and conditions for protecting and deprotecting are known in the art and are described, for example, in Greene and Wuts, “Protective Groups in Organic Synthesis”, John Wiley & Sons (1991), the entire teachings of which are incorporated herein by reference. Specific examples of suitable hydroxyl protecting groups include, but are not limited to substituted methyl ethers (e.g., methoxymethyl, benzyloxymethyl) substituted ethyl ethers (e.g., ethoxymethyl, ethoxyethyl) benzyl ethers (benzyl, nitrobenzyl, halobenzyl) silyl ethers (e.g., trimethylsilyl), esters, and the like.

As used herein, a “halogenating agent” is a compound known to the art that can substitute a halogen for a —H or other group. For example, in various embodiments, a halogenating agent can be bromine, chlorine, N-chlorosuccinimide, N-bromosuccinimide, or the like, particularly N-bromosuccinimide.

As used herein, the Prilezhaev reaction means the formation of epoxides by the reaction of alkenes with peracids . See, for example, N. Prilezhaev, Ber. 42, 4811 (1909); D. Swern, Chem. Rev. 45, 16 (1949); Org. React. 7, 378 (1953); H. O. House, Modern Synthetic Reactions (W. A. Benjamin, Menlo Park, Calif., 2nd ed., 1972) pp 302-319; D. I. Metelitra, Russ. Chem. Rev. 41, 807 (1972); and D. Schnurgfeil, Z. Chem. 20, 445 (1980). The entire teachings of these references are incorporated herein by reference.

As used herein, a “drug metabolizing enzyme” is an enzyme that, in vivo or in vitro, can chemically modify CsA, ISA247, or metabolites thereof. Typically, a drug metabolizing enzyme is derived from an animal or microorganism, e.g., a mammal, for example, by homogenizing mammalian cells (e.g., liver cells) to produce a microsomal preparation which contains the drug metabolizing enzymes. In particular embodiments, drug metabolizing enzymes include one or more members of the cytochrome P450 family of enzymes.

As used herein, an “energy source” includes one or more compounds that can be used by a drug-metabolizing enzyme to provide energy to chemically modify ISA247 to produce a metabolite of ISA247. For example, an energy source can be a carbohydrate, e.g., in particular embodiments, glucose-6-phosphate, isocitrate, and the like. An enzyme to facilitate digestion of the energy source can be included in a microsome preparation, for example, glucose-6-phosphate dehydrogenase, isocitrate dehydrogenase, and the like.

As used herein, an “electron donating species” is a compound that can be used by a drug-metabolizing enzyme to as a source of redox electrons to chemically modify ISA247 to produce a metabolite of ISA247. For example, typical electron donating species include nicotinamide adenine dinucleotide (NADH) and nicotinamide adenine dinucleotide phosphate (NADPH).

As used herein, “isolated” means that a compound, e.g., a disclosed metabolite of ISA247, is separated from a biological system, e.g., a mammal, cells of a microbial cell culture, or the like. Typically, an isolated compound is also purified, e.g., by chromatographic separation, crystallization, affinity purification, or other means known to the art. For example, in particular embodiments, the disclosed ISA247 metabolites can be purified by high pressure liquid chromatography.

As used herein, “N-demethylation” means the removal of a methyl group from an amino acid nitrogen. An “amino acid nitrogen” is the nitrogen in the backbone of the amino acid, not a nitrogen in an amino acid side chain.

As used herein, “glucuronide formation” means the formation of a glucuronide ISA247 metabolite by linking glucuronic acid to ISA247 or another ISA247 metabolite via a glycosidic bond.

As used herein, “glycosylation” means the bonding of a saccharide to a hydroxyl group of an ISA247 metabolite to form a glycosylated ISA247 metabolite. A saccharide (or glycosylation residue) can have one or more sugars, e.g., including disaccharides, oligosaccharides, polysaccharides, and the like. Typical sugars included in glycosylation residues are glucose, mannose, and N-acetyl glucosamine.

As used herein, an aliphatic group is a straight chained, branched or cyclic non-aromatic hydrocarbon which is completely saturated or which contains one or more units of unsaturation. An alkyl group is a saturated aliphatic group. Typically, a straight chained or branched aliphatic group has from 1 to about 10 carbon atoms, preferably from 1 to about 4, and a cyclic aliphatic group has from 3 to about 10 carbon atoms, preferably from 3 to about 8. An aliphatic group is preferably a straight chained or branched alkyl group, e.g., methyl, ethyl, n-propyl, iso-propyl, n-butyl, sec-butyl, tert-butyl, pentyl, hexyl, pentyl or octyl, or a cycloalkyl group with 3 to about 8 carbon atoms. C1-C4 straight chained or branched alkyl or alkoxy groups or a C3-C8 cyclic alkyl or alkoxy group (preferably C1-C4 straight chained or branched alkyl or alkoxy group) are also referred to as a “lower alkyl” or “lower alkoxy” groups; such groups substituted with —F, —Cl, —Br, or —I are “lower haloalkyl” or “lower haloalkoxy” groups; a “lower hydroxyalkyl” is a lower alkyl substituted with —OH; and the like.

As used herein, an “alkylene group” is a linking alkyl chain represented by —(CH₂)ₙ—, wherein n is an integer from 1-10, preferably 1-4.

As used herein, the term “aryl” means C6-C14 carbocyclic aromatic groups such as phenyl, biphenyl, and the like. Aryl groups also include fused polycyclic aromatic ring systems in which a carbocyclic aromatic ring is fused to other aryl, cycloalkyl, or cycloaliphatic rings, such as naphthyl, pyrenyl, anthracyl, and the like.

As used herein, the term “heteroaryl” means 5-14 membered heteroaryl groups having 1 or more O, S, or N heteroatoms. Examples of heteroaryl groups include imidazolyl, isoimidazolyl, thienyl, furanyl, fluorenyl, pyridyl, pyrimidyl, pyranyl, pyrazolyl, pyrrolyl, pyrazinyl, thiazoyl, isothiazolyl, oxazolyl, isooxazolyl, 1,2,3-trizaolyl, 1,2,4-triazolyl, imidazolyl, thienyl, pyrimidinyl, quinazolinyl, indolyl, tetrazolyl, and the like. Heteroaryl groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazolyl, benzoisothiazolyl, benzooxazolyl, benzoisooxazolyl, benzimidazolyl, quinolinyl, isoquinolinyl and isoindolyl.

As used herein, non-aromatic heterocyclic groups are non-aromatic carbocyclic rings which include one or more heteroatoms such as N, O, or S in the ring. The ring can be five, six, seven or eight-membered. Examples include oxazolinyl, thiazolinyl, oxazolidinyl, thiazolidinyl, tetrahydrofuranyl, tetrahydrothiophenyl, morpholino, thiomorpholino, pyrrolidinyl, piperazinyl, piperidinyl, thiazolidinyl, cyclic saccharides (e.g., glucose, mannose, galactose, allose, altrose, gulose, idose, talose, and the like, in pyranose and furanose forms) and the like.

Suitable optional substituents for a substitutable atom in alkyl, cycloalkyl, aliphatic, cycloaliphatic, heterocyclic, benzylic, aryl, or heteroaryl groups are those substituents that do not substantially interfere with the pharmaceutical activity of the disclosed ISA427 metabolites. A “substitutable atom” is an atom that has one or more valences or charges available to form one or more corresponding covalent or ionic bonds with a substituent. For example, a carbon atom with one valence available (e.g., —C(—H)═) can form a single bond to an alkyl group (e.g., —C(-alkyl)=), a carbon atom with two valences available (e.g., —C(H₂)—) can form one or two single bonds to one or two substituents (e.g., —C(alkyl)(H)—, —C(alkyl)(Br))—,) or a double bond to one substituent (e.g., —C(═O)—), and the like. Substitutions contemplated herein include only those substitutions that form stable compounds.

For example, suitable optional substituents for substitutable carbon atoms (e.g., the substituents represented by R⁵—R¹⁰) include —F, —Cl, —Br, —I, —CN, —NO₂, —OR^{a}, —C(O)R^{a}, —OC(O)R^{a}, —C(O)OR^{a}, —SR^{a}, —C(S)R^{a}, —OC(S)R^{a}, —C(S)OR^{a}, —C(O)SR^{a}, —C(S)SR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), SO₂N(R^{a}R^{b}), —NRC^{c}(O)R^{a}, —NR^{c}C(O)OR^{a}, —NR^{c}C(O)N(R^{a}R^{b}), —C(NR^{c})—N(R^{a}R^{b}), —NR^{d}—C(NR^{c})—N(R^{a}R^{b}), —NR^{a}N(R^{a}R^{b}), —CRC═CR^{a}R^{b}, —C≡CR^{a}, ═O, ═S, ═CR^{a}R^{b}, ═NR^{a}, ═NOR^{a}, ═NNR^{a}, optionally substituted alkyl, optionally substituted cycloalkyl, optionally substituted aliphatic, optionally substituted cycloaliphatic, optionally substituted heterocyclic, optionally substituted benzyl, optionally substituted aryl, and optionally substituted heteroaryl, wherein R^{a}—R^{d} are each independently —H or an optionally substituted aliphatic, optionally substituted cycloaliphatic, optionally substituted heterocyclic, optionally substituted benzyl, optionally substituted aryl, or optionally substituted heteroaryl, or, —N(R^{a}R^{b}), taken together, is an optionally substituted heterocyclic group.

Suitable substituents for nitrogen atoms (for example, the amino acid nitrogens in amino acid residues 1-11 in the disclosed ISA247 metabolites) having two covalent bonds to other atoms include, for example, optionally substituted alkyl, optionally substituted cycloalkyl, optionally substituted aliphatic, optionally substituted cycloaliphatic, optionally substituted heterocyclic, optionally substituted benzyl, optionally substituted aryl, optionally substituted heteroaryl, —CN, —NO₂, —OR^{a}, —C(O)R^{a}, —OC(O)R^{a}, —C(O)OR^{a}, —SR^{a}, —S(O)R^{a}, —SO₂R^{a}, —SO₃R^{a}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a}, —NR^{c}C(O)N(R^{a}R^{b}), and the like.

A nitrogen-containing heteroaryl or non-aromatic heterocycle can be substituted with oxygen to form an N-oxide, e.g., as in a pyridyl N-oxide, piperidyl N-oxide, and the like. For example, in various embodiments, a ring nitrogen atom in a nitrogen-containing heterocyclic or heteroaryl group can be substituted to form an N-oxide.

As used herein, the term “pharmaceutically acceptable” means that the materials (e.g., compositions, carriers, diluents, reagents, salts, and the like) are capable of administration to or upon a mammal.

Also included in the present invention are pharmaceutically acceptable salts of the disclosed ISA247 metabolites. These metabolites can have one or more sufficiently acidic protons that can react with a suitable organic or inorganic base to form a base addition salt. When it is stated that a compound has a hydrogen atom bonded to an oxygen, nitrogen, or sulfur atom, it is contemplated that the compound also includes salts thereof where this hydrogen atom has been reacted with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

For example, pharmaceutically acceptable salts can include those formed by reaction of the disclosed ISA247 metabolites with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation). “Pharmaceutically acceptable” means that the cation is suitable for administration to a subject. Examples include Li⁺, Na⁺, K⁺, Mg²⁺, Ca²⁺ and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically acceptable cation is Li⁺, Na⁺, K⁺, NH₃(C₂H₅OH)⁺or N(CH₃)₃(C₂H₅OH)³⁰ .

Pharmaceutically acceptable salts of the disclosed ISA247 metabolites with a sufficiently basic group, such as an amine, can be formed by reaction of the disclosed ISA247 metabolites with an organic or inorganic acid to form an acid addition salt. Acids commonly employed to form acid addition salts from compounds with basic groups can include inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Also included are pharmaceutically acceptable solvates. As used herein, the term “solvate” means a compound of the present invention or a salt thereof, that further includes a stoichiometric or non-stoichiometric amount of solvent, e.g., water or organic solvent, bound by non-covalent intermolecular forces.

Also included are pharmaceutical compositions comprising the disclosed ISA247 metabolites. A “pharmaceutical composition” comprises a disclosed ISA247 metabolite in conjunction with an acceptable pharmaceutical carrier as part of a pharmaceutical composition for administration to a subject. Formulation of the compound to be administered will vary according to the route of administration selected (e.g., oral, I.V., parenteral, or topical administration solution, emulsion, capsule, cream, ointment, and the like ). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrasn) are known in the art (Baker, et al., “Controlled Release of Biological Active Agents”, John Wiley and Sons, 1986).

It will also be understood that certain of the disclosed ISA247 metabolites can be obtained as different stereoisomers (e.g., diastereomers and enantiomers) and that the invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

Identifying and Isolating ISA247 Metabolites from Human Whole Blood, Urine or Bile

Using organic extractions on these fluids, metabolites were extracted, dried, reconstituted in methanol and identified using chromatographic techniques coupled with mass spectrometry.

Chemical Synthesis of ISA247 Metabolites

Metabolites of ISA247 can be prepared by chemical synthesis. The monohydroxylated metabolite C_{S}A-A1 of cyclosporine A has been synthesized by M. K. Eberle and F. Nuninger, as reported in “Synthesis of the main metabolite (OL-17) of cyclosporin A,” J. Org. Chem. Vol. 57, No. 9, pp. 2689-2691 (1992). Eberle et al. have noted that cyclosporine A is metabolized in humans and animals to a cyclosporin whereby the original allylic methyl group (on the side chain of the 1-amino acid residue) is oxidized to the corresponding allyl alcohol. Eberle et al. have “attempted to mimic this metabolic pathway in vitro” by protecting the β-alcohol of the amino acid-1 residue, and then subjecting the resulting acetyl-cyclosporine A (acetyl—C_{S}A) to the conditions of an allylic bromination by treating the acetyl-C_{S}A with N-bromosuccinimide (NBS). The product from that step was then heated in the presence of tetrabutylammonium acetate, which affects the exchange of the bromide by the acetate. Finally, the synthesis was completed by performing a transesterification of the acetate in methanol, in the presence of sodium methoxide, to exchange the acetate group with an alcohol function. This conversion of cyclosporine A to the OL-17 metabolite was reportedly obtained in a 28% yield following reversed phase column chromatography.

A similar synthetic pathway may be used to produce ISA247 metabolites, such as IM4, IM6 and IM9, or for that matter, any metabolite in which an alkyl carbon, such as a pendant methyl group, is hydroxylated. For example, the hydroxylation may occur at the γ-CH₃ of the side chain of the amino acid-1 residue of ISA247, thus resulting in a 1,2 diol across the β-γ-CH₃ carbons of the side chain.

In other embodiments, the metabolites IM4, IM6, and l[M9 may be synthesized by 1) protecting the β-alcohol of the amino acid-1 residue of the parent compound ISA247 to form a protected-ISA247 compound, 2) treating the protected-ISA247 with a halogenating agent, such as N-bromosuccinimide (NBS), to form a protected-ISA247 that is halogenated (which in one embodiment is brominated) at the γ-carbon of the side chains of the 4, 6, or 9-amino acid residues, 3) heating the product of the previous step in the presence of a substituting reagent, such as tetrabutylammonium acetate, to form an acetate-containing product, and 4) performing a transesterification to exchange the acetate moiety of the acetate-containing product of the previous step with an alcohol moiety to form the hydroxylated metabolite. The general chemical concepts of a monohydroxylation of a side chain of a residue other than the amino acid-1 residue of ISA247 may be similar to that of the above-described monohydroxylation of the side chain of the amino acid-1 residue of C_{S}A. However, it is contemplated that substantially different synthetic strategies will have to be employed to obtain metabolites which are hydroxylated at the amino acid-1 residue of ISA247 in view of the conjugated diene system that is present in ISA247, but not C_{S}A.

In an alternative embodiment directed toward chemically synthesizing ISA247 metabolites, the alkene moieties of the side chain of the amino acid-1 of ISA247 may be converted to diols either directly, or through an epoxide intermediate. For example, the conversion of alkenes to epoxides is known in the chemical literature. In one embodiment that uses the Prilezhaev reaction, an olefin can be treated with a suitable oxidizing agent resulting in the addition of oxygen across the carbon-carbon double bond of the olefin, thus forming an epoxide. A common oxidizing agent that may be employed in this reaction is a peracid, and in one embodiment of the present invention, m-chloroperbenzoic acid (MCPBA) is a preferred reagent. Other peracids, such as peracetic, triflouroperacetic, perbenzoic, and 3,5-dinitroperbenzoic acid may also be employed. It is contemplated that treatment of the alkene-containing cyclosporins of the present invention with hydrogen peroxide, alkyl peroxides or oxygen, may also result in epoxide formation.

The epoxide-containing metabolites of the present invention may then undergo nucleophilic attack by water to form 1,2-diols. The reaction may be catalyzed by either acids or bases. In one embodiment of the present invention, perchloric acid is a preferred reagent, but other acid catalysts, such as Nafion-H or formic acid, may also be effective. In another embodiment, an alkaline hydrolysis of the epoxide-metabolite in dimethyl sulfoxide may be carried out under basic conditions. Catalysis of epoxide hydrolysis by the enzyme microsomal epoxide hydrolase is contemplated as yet another method. This method offers particular advantages in that the reaction may introduce a degree of stereoselectivity to the reaction products.

Alternately, an alkene may be converted directly to a 1,2-diol by a number of different reagents. Osmium tetroxide and alkaline potassium permanganate may give syn addition. Similarly, reagents such as hydrogen peroxide or t-butyl hydroperoxide, in the presence of catalytic osmium tetroxide, also may give syn addition. Conversely, anti-addition may be possible through treatment with, for example, hydrogen peroxide and formic acid, or monopersuccinic acid. Treatment of an alkene with iodine and silver benzoate or silver acetate results in intermediate diesters which can be readily hydrolyzed to give 1,2-diols. Similarly, oxidation with lead tetraacetate or thallium acetate gives hydrolyzable bisacetates of diols. Thus, it will be understood by those skilled in the art that numerous approaches may be taken for the direct conversion of olefins to 1,2-diols.

The above-mentioned synthetic approaches for converting alkenes to 1,2-diols are contemplated to be useful in the synthetic preparation of diol metabolites of ISA247. The alkenes of the conjugated diene moiety of the side chain of the amino acid-1 residue of ISA247 may be chemically converted into diols, either directly, or through intermediates, such as epoxides. The resulting compounds may then be compared and matched to metabolites produced by other techniques, such as those produced by a rabbit or dog microsomal system. It is contemplated by the inventors that epoxide intermediates formed from ISA247 are likely to possess pharmacological activity, and may therefore be of interest as potential therapeutic agents.

Since ISA247 differs structurally from cyclosporine A only in the chemical composition of amino acid-1, certain metabolites of cyclosporine A may be of use as intermediates for the synthesis of ISA247 metabolites. For example, cyclosporine A metabolites, such as AM4n (N-demethylation of the amino acid-4 residue) and AM9 (hydroxylation on the side chain of the amino acid-9 residue), may be converted to the analogous ISA247 metabolites via the same chemical process that converts cyclosporine A to ISA247.

Preparing ISA247 Metabolites via a Mammalian Microsomal System

Cyclosporine A and ISA247 are metabolized extensively by microsomal systems from humans and other animals (Christians, 1993). In human liver microsomal preparations, the conversion of cyclosporin to its metabolites is NADPH dependent. The metabolism can be inhibited by carbon monoxide, ketoconazole, cimetidine, and SKF525A. As these inhibitors are known to be specific cytochrome P-450 inhibitors, it has been suggested that metabolism of cyclosporin is mediated through the monooxygenase function of the cytochrome P-450 system. The inventors have shown previously that ISA247 is also metabolized by the cytochrome P-450 enzyme system.

Microsome systems are well-known in the art and are prepared by homogenizing the appropriate tissue (which can include tissue from liver, kidney, gastrointestinal tissue and the like from mammals such as rabbits, dogs, pigs, cows, sheep, primates, rats, mice and the like) and centrifuging at 100,000×g, yielding a microsomal pellet, which when reconstituted can be used to mimic in-vivo metabolism of ISA247. Once metabolites are created in the microsome preparation, ISA247 metabolites can be isolated and analyzed using HPLC/MS or NMR, or other techniques.

Preparing ISA247 Metabolites via Biotransformation Methods with Microorganisms

In embodiments of the present invention, metabolites of ISA247 may be prepared utilizing cultures of microorganisms and biotransformation. It is possible to produce metabolites of ISA247 which correspond to ISA247 metabolites in humans using biotransformation because certain microorganisms have the ability to mimic the activity of the human cytochrome P-450 system.

Exemplary microorganisms that may be useful for biotransformation methods include Actinoplanes sp. (e.g., ATCC No. 53771, available from American Type Culture Collection Manassas, Va. USA), Streptomyces griseus (e.g., ATCC 13273), Saccharopolyspora erythraea (e.g., ATCC No. 11635), and Streptomyces setonii (e.g., ATCC No. 39116). Other useful microorganisms may include Amycolata autotrophica (e.g., ATCC No. 35204, also known as Pseudonocardia autotrophica), Streptomyces californica (e.g., ATCC No. 15436), Saccharopolysora hirsute (e.g., ATCC No. 20501), Streptomyces lavandulae (e.g., ATCC 55209), Stretomyces aureofaciens (e.g., ATCC 10762), Streptomyces rimosus (e.g., ATCC 28893, also known as Penicillium expansum), Bacillus subtillis (e.g., ATCC 55060), and Nocardia asteroids (e.g., ATCC 3318, also known as Nocardia farcinica). In various embodiments, useful microorganisms can include Curvularia lunata (e.g., ATCC 12017, or UAMH 9191, available from University of Alberta Microfungal Collection and Herbarium, Edmonton, Alberta, Canada), Cunninghamella echinulata var. elegans (e.g., UAMH 7370, ATCC 36112), Curvularia echinulata var. blakesleena (e.g., UAMH 8718, ATCC 8688a), Cunninghamella echinulata var. elegans (e.g., UAMH 7369, ATCC 26269), Beauvaria bassiana (e.g., UAMH 8717, ATCC 7159), Actinomycetes (e.g., ATCC 53828), Actinoplanes (e.g., ATCC 53771), Cunninghamella echinulata (e.g., UAMH 4144, ATCC 36190), Cunninghamella echinulata (e.g., UAMH 7368, ATCC 9246), Cunninghamella bainiere (echinulata) (e.g., UAMH 4145, ATCC 9244) and Saccharopolyspora erythrae (e.g., ATCC 11635).

To the inventors' knowledge, conventional biotransformation methods have not been successful at producing metabolites of cyclosporin and ISA247, perhaps because of the lipophilic nature of these compounds. Without wishing to be bound by any theory, it is believed that a hydrophobic compound, such as ISA247, may tend to adhere to the surfaces of filters, columns, and other hardware used to carry out the culture and process the product metabolites, e.g., ISA247 may tend to adhere to the surfaces of the filters used to aseptically add the drug to the culture media.

In embodiments of the present invention, metabolites of ISA247, including metabolites of C_{S}A and ISA247, may be produced by preparing a mixture of the drug and at least one surfactant, and adding the drug-surfactant mixture directly to the growth media of a microorganism. The surfactant may be sterilized. When this step is followed, the present inventors have found that the biotransformation becomes a more productive one. In particular embodiments of this method, the surfactant is a Tween.

Suitable surfactants may be able to withstand autoclaving prior to being introduced into a microbial growth environment. Suitable surfactants are biocompatible surfactants and include, but are not limited to, nonionic surfactants such as polyethylene glycols, for example PEG 300, PEG 400, PEG 600 (also known as Lutrol® E 300, Lutrol® E 400, Lutrol® E 600 Lutrol® F 127, and Lutrol® F 68 from BASF); caprylocaproyl macrogol-8 glycerides such as Labrasol® (Gatte Fosse, Cedex France); polyoxyethylene sorbitan fatty acid esters such as Tween® 20, Tween® 21, Tween® 40, Tween® 80, Tween® 80K, Tween® 81 and Tween® 85 (ICI Americas Inc., Bridgewater N.J., obtained from Aldrich Chemical Company Inc., Milwaukee Wis.); glycerin (BDH Fine Chemicals, Toronto Ont.); castor oil (Wiler Fine Chemicals Ltd, London Ont.); Isopropyl myristate (Wiler Fine Chemicals Ltd, London Ont.); Cremaphor EL (Sigma Chemical, St Louis Mo.); and poloxamers such as Pluronics® F127 and Pluronics® L108 (BASF). Other surfactants that may be used include those that can act as lubricants or emulsifiers such as tyloxapol [4-(1,1,3,3-tetramethylbutyl)phenol polymer with formaldehyde and oxirane]; polyethoxylated castor oils such as Cremaphor A25, Cremaphor A6, Cremaphor EL, Cremaphor ELP, Cremaphor RH from BASF and Alkamuls EL620 from Rhone Poulenc Co; polyethoxylated hydrogenated castor oils, such as HCO-40; and polyethylene 9 castor oil.

Other surfactants that may be used include; polysorbate 20, polysorbate 60, and polysorbate 80; Cremaphor RH; poloxamers; Pluonics L10, L31, L35, L42, L43, L44, L61, L62, L63, L72, L81, L101, L121, L122; PEG 20 almond glyceride; PEG 20 corn glyceride; and the like. Suitable surfactants also include alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene-polyoxypropylene block copolymers; polyglycerol fatty acid esters; polyoxyethylene glycerides; polyoxyethylene sterols; polyoxyethylene vegetable oils; polyoxyethylene hydrogenated vegetable oils; polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction mixtures of polyols such as PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, sucrose monostearate, sucrose monolaurate, sucrose monopalmitate, PEG 10-100 nonyl phenol series, PEG 15-100 octyl phenol series, a poloxamer; PEG-35 castor oil, PEG-40 hydrogenated castor oil, PEG-60 corn oil, PEG-25 glyceryl trioleate, PEG-6 caprate/caprylate glycerides, PEG-8 caprate/caprylate glycerides, polysorbate 20, polysorbate 80, tocopheryl PEG-1000 succinate, and PEG-24 cholesterol, a poloxamer. In addition, oils such as almond oil; babassu oil; borage oil; blackcurrant seed oil; canola oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides may be used. In addition, a mixture of surfactants and/or oils and/or alcohols may be used.

In certain embodiments of the present invention, the parent compound is mixed with an alkanol such as ethanol and a suitable nonionic surfactant before addition to an actively growing microbial culture. If the parent compound is mixed with an alcohol, the alcohol may be ethanol. Additional suitable alcohols include: methanol, and other suitable alcohols well known in the art.

After addition of the parent compound-surfactant mixture to the bioreaction mixture that contains the microorganism in growth medium, the bioreaction is allowed to proceed for a time and under conditions which permit the parent compound to be metabolized. After the desired time, the metabolites are extracted from the bioreaction mixture, purified by separation, for example by chromatography such as high pressure liquid chromatography and mass spectral analysis (HPLC-MS). Nuclear magnetic resonance analysis may be used to verify that the individual metabolites have been isolated from one another and to verify the structure thereof.

In some embodiments of the present invention, ISA247 in ethanol is mixed with glycerin and then added to a biotransformation system containing Saccharopolyspora erytheraea ATCC 11635. In other embodiments PEG 400 is mixed with ISA247 in ethanol prior to the addition of ISA247 to the biotransformation system. In other embodiments castor oil is mixed with ISA247 in ethanol prior to the addition of ISA247 to the biotransformation system. In other embodiments isopropyl myristate is mixed with ISA247 in ethanol prior to the addition of ISA247 to the biotransformation system. In other embodiments Cremaphor is mixed with ISA247 in ethanol prior to the addition of ISA247 to the biotransformation system. In other embodiments Labrasol® is mixed with ISA247 in ethanol prior to the addition of ISA247 to the biotransformation system. In other embodiments Tween 40 is mixed with ISA247 in ethanol prior to the addition of ISA247 to the biotransformation system.

Analysis and Elucidation of ISA247 Metabolites

Metabolites of ISA247 were separated by their chemical characteristics and their kinetic parameters using high performance liquid chromatography (HPLC) coupled with mass spectrometry (HPLC-MS or LC-MS/MS).

The inventors have developed qualitative and quantitative methods for analyzing ISA247 metabolites using liquid chromatography techniques in conjunction with mass spectrometry. These methods are capable of isolating and characterizing ISA247 metabolites produced in vitro and in vivo, and are also well-suited for the quantitative monitoring of ISA247 metabolites in whole blood or other body fluids as part of pharmacokinetic studies.

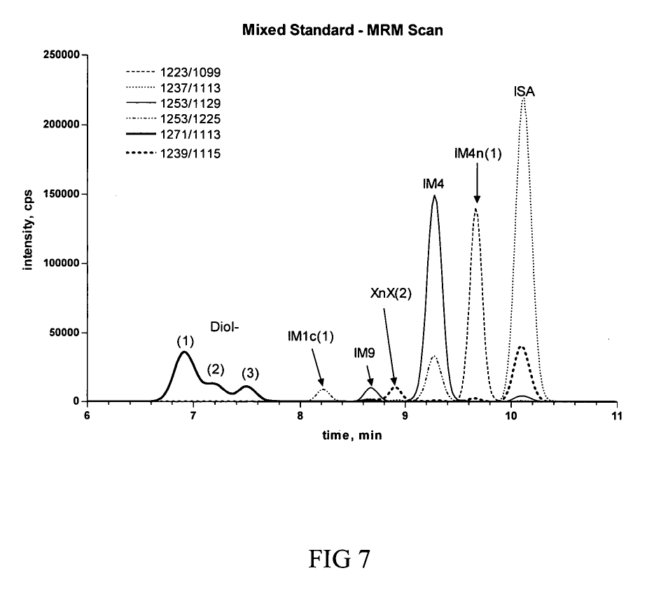

Identification of analytes is done on the basis of retention times obtained from high-pressure liquid chromatography (HPLC) data, and structure-specific ion fragment information obtained from electrospray ionization (ESI) mass spectral data. FIG. 6 is an LCMS trace in MRM mode (Multiple Reaction Monitoring mode) using a SCIEX™ triple quad mass spectrometer.

The HPLC-MRM scan of FIG. 6 shows a typical ISA247 metabolic profile isolated from human whole blood and is consistent with the LC-MS fragmentation profile seen for ISA247 extracts prepared from liver microsomes or from biotransformation sources. The MRM scan of FIG. 6 identifies four peaks in the 1271/1113 range, indicating at least four different diol peaks (Diol(1), Diol(2), Diol(3) and Diol(4)), 3 small peaks in the 1239/1115 range labeled IMXnX(2), IMXnX(4), and IMXnX(6), 4 peaks in the 1253/1225 range labeled IMX(1), IM9, IM4 and IMX(2), two peaks in the 1223/1099 range labeled IM4n and IMXn(2), and a large peak at 1237/1113 for ISA247, as identified by non-metabolized standards. Several additional peaks are shown, but not labeled.

The structures of HPLC purified materials of transformed ISA247 were further elucidated using Nuclear Magnetic Resonance (NMR) techniques using the following general conditions. ID and 2D NMR spectra were recorded in benzene-d6 on a Varian Inova 800 and/or 500 MHz, and/or a Varian Mercuryplus 400 MHz spectrometers at 25˜27 ° C. The benzene signal was set at δ 7.15 ppm for ¹H-NMR and at δ 128.06 ppm for ¹³C-NMR as reference. Sample concentrations of 0.5˜1 mg/˜0.7 ml of benzene d₆ were used. The obtained spectra were analyzed using 2D NMR Processor software of ACD/Labs (Advanced Chemistry Development Inc., Toronto, Canada).

Transformation at the Amino Acid-1: Diols, Cyclics and Epoxides

As shown in FIG. 6, metabolites isolated from human whole blood show that a series of main metabolite peaks occur at a retention time of about 6.5 to 8 minutes, and a parent ion/fragment ion pair of 1271/1113 m/z. This indicates an addition of 34 mass units to the parent ISA247 mass of 1237 m/z. Furthermore, it may be concluded that the chemical modification comprising the metabolite is localized to amino acid-1 residue since the fragment ion has a mass of 1113 m/z. From this information, it was possible to hypothesize that this modification is most likely a diol formation at the diene region of the amino acid-1 between the ε carbon and the θ carbon positions.

Diol metabolites can be isolated from HPLC for further structural analysis. It is important to note that, depending on the source of the material, whether from chemical synthesis, or isolated from biotransformation, microsomes or blood or urine, the peaks that appear in the HPLC may be different. For example, chemical synthesis may create diols which exist as both R and S diastereomers of a diol structure, while enzymatic metabolism may form one diastereomer of a diol structure, and not the other. An enzyme may prefer substrates in one orientation and not the other, and therefore, may produce one diastereomer, and not the other. Diastereomers of diol metabolites may appear as different peaks on HPLC because they may have different chemical properties which cause them to migrate differently through a column. Therefore, HPLC traces for metabolites which are produced by different means, by chemical synthesis and biotransformation, for example, may have different peaks. Therefore, it is important to note that the HPLC shown in FIG. 6 may not be representative of an HPLC trace for metabolites which are not isolated from human whole blood. In addition, it is important to note that the nomenclature used for diols, i.e., IM1-d-1, does not necessarily correspond to the HPLC peaks shown in FIG. 6. This nomenclature should be considered in light of the structures as presented in Table 1.

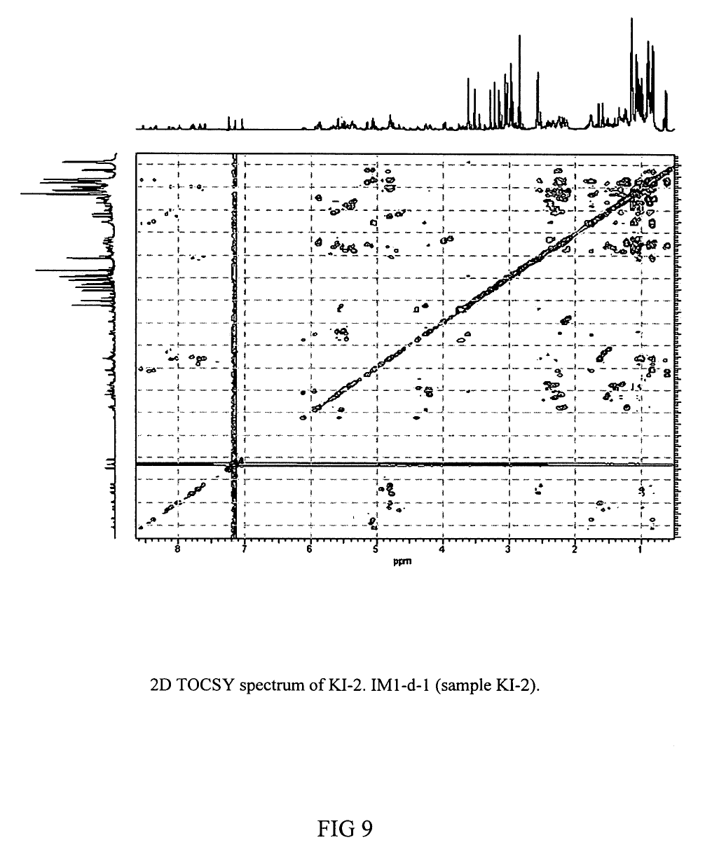

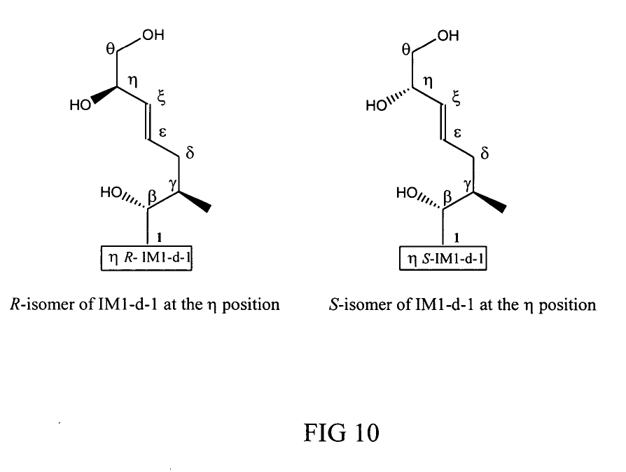

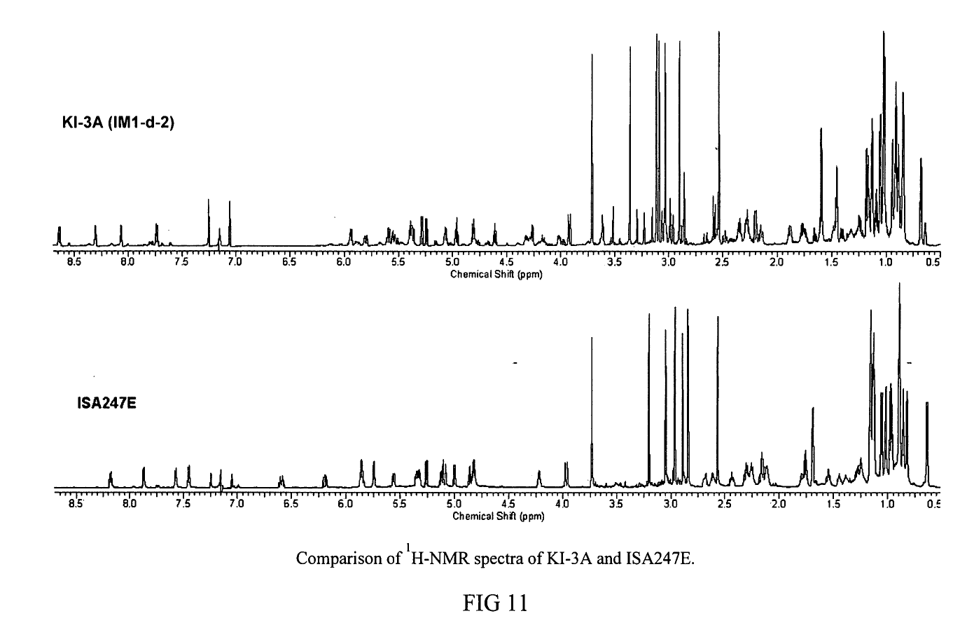

¹H-NMR and 2D NMR techniques were employed to further elucidate the structure(s) of diol metabolites of ISA247 produced by chemical synthesis, biotransformation, or isolated from blood or urine, and isolated using HPLC-MS. FIG. 8 shows the ¹H-NMR spectrum from the HPLC purified product of the IMN1-d-1 compound, where the product was made by the microorganism biotransformation method described above (also labeled KI-2), compared to the ¹H-NMR spectra for the E and Z isomers of ISA247. Comparison of the three spectra reveal changes in the IM1-diol-1 or IM1-d-1 ¹H-NMR at the diene region between about δ 6 and 7 ppm, indicative of an aa-1 side chain transformation. The amide NH proton and the N-methyl proton region indicate that the metabolite is a mixture of two major products with minor contaminations, assuming four NH and seven N-methyl signals arise from each of the major products. This assumption was substantiated by the correlation spectrum utilizing the amide proton signals. The amide NH proton cross-peak correlations with relevant ax protons and side chain methyl groups were obtained from the 2D TOCSY spectrum of IM1-d-1 (FIG. 9). Based on the NMR analysis, it was found that this diol fraction (KI-2) is mainly composed of a 1:1 mixture of two diastereomers, differing in the stereochemistry at the η carbon of the amino acid-1 (aa-1). The trans double-bond configuration was determined from the coupling constant for both diastereomers having J_{εζ}=15.0 Hz. The proposed structures for IM1-d-1 are shown in FIG. 10.

FIG. 10 shows that the structure of IM1-d-1 is a diol at the η and θ positions of the amino acid-1 (aa-1) side chain of trans-ISA247. Because the η position is a chiral center, the IM1-d-1 compound can exist as two diastereomers, differing in the configuration at the η carbon of the amino acid-1, as shown in FIG. 10. The two diastereomers present a different NMR spectrum because each diastereomer is a different chemical entity with specific physicochemical properties.

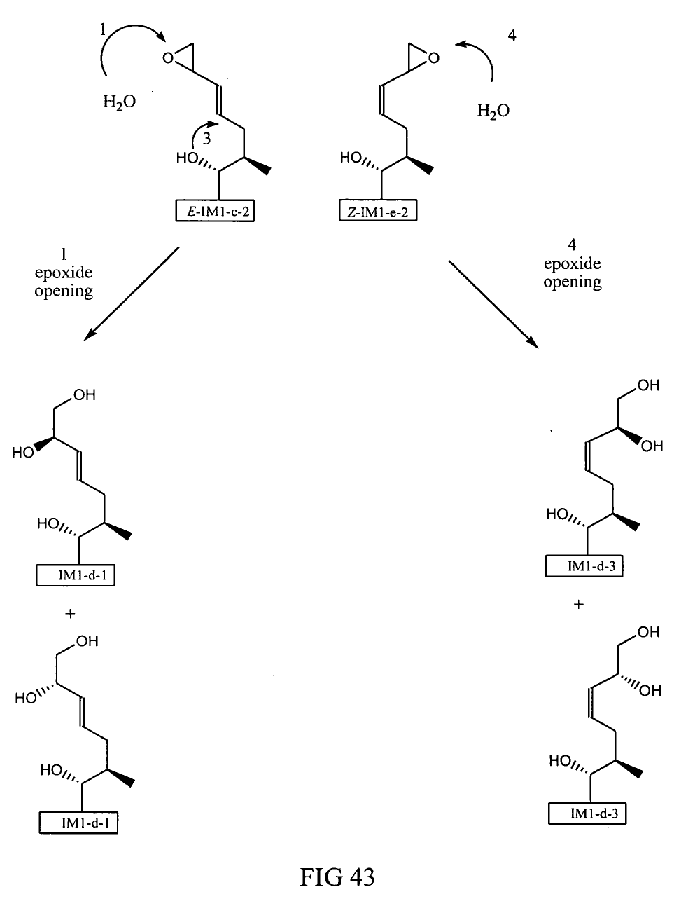

Surprisingly, the NMR studies showed that a starting material of 50:50 cis:trans ISA247, where the metabolite is made using a biotransformation method (using fungi, the KI-2 sample), results in a mixture of IM1-d-1 diastereomers, shown in FIG. 10, in the trans configuration, in a 1:1 ratio of diastereomers. However, a starting material of mostly trans-ISA247, prepared by chemical synthesis (the KI-2A sample), when isolated using HPLC-MS and studied using NMR, results in a mixture of IM1-d-1 diastereomers in a 3:2 ratio. IM1-d-1 is a metabolite formed from trans-ISA247, as shown in FIGS. 42 and 43. While the difference in the isomeric ratio detected in the KI-2A NMR spectrum made it possible to obtain the proton chemical shift assignment for each diastereomer, it was not possible to assign one of the structures as shown in FIG. 10 to the ratio determination by NMR. This discrepancy indicates the possibility of a preferential production of one diatereomeric metabolite over another, depending on whether the metabolites were produced using naturally occurring enzymes (from liver or from fungus) or using chemical reactions in the laboratory. For example, during a chemical synthesis, an orientation of chemical intermediate may produce a diasteromeric mixture that is richer in one isomer than another. Alternatively, the biotransformation process may create diastereomeric metabolites, and one of those diastereomers may be preferentially further processed into another metabolite, different from the IM1-d-1, which skews the ratio of products that are present in the corresponding peak in the HPLC. Alternatively, the chemical process or the biotransformation process may create a diastereomer that is further processed into another metabolite, different from the IM1-d-1 compound, which skews the ratio of products that are present in a specific peak using HPLC. Table 1 shows the chemical shift assignments for IM1-d-1, based on the above NMR analysis. While this table represents one compound which was identified by NMR, more than one compound was apparent in this sample. Chemical shift information for the second compound are not shown in Table 1. However, the two terminal diol diastereomers, IM1-d-1, are shown in FIG. 43.

TABLE 1
IM1-d-1 (KI-2A sample) major

| | | Chemical shift |
|---|---|---|
| Amino acid | Hs | (ppm)³⁾ |
| Amino acid-1 | | |
| CH(α) | 1 | 5.60 |
| CH(β) | 1 | 4.21 |
| CH(γ) | 1 | 2.13 |
| γ-CH3 | 3 | 1.08 |
| CH(δ1) | 1 | 2.38 |
| CH(δ2) | 1 | 2.35 |
| CH(ε) | 1 | 5.94⁴⁾ |
| CH(ζ) | 1 | 5.53⁴⁾ |
| CH(η) | 1 | 4.28 |
| CH(θ1) | 1 | 3.63 |
| CH(θ2) | 1 | 3.63 |
| N—Me | 3 | 3.62 |
| Amino acid-2 | | |
| CH(α) | 1 | 5.07 |
| CH(β1) | 1 | 1.77 |
| CH(β2) | 1 | 1.77 |
| CH3(γ) | 3 | 0.86 |
| NH | 1 | 8.35 |
| Amino acid-3 | | |
| CH(α1) | 1 | 3.98 |
| CH(α2) | 1 | 2.17 |
| N—Me | 3 | 3.07 |
| Amino acid-4 | | |
| CH(α) | 1 | 5.60 |
| CH(β1) | 1 | 2.24 |
| CH(β2) | 1 | 1.54 |
| CH(γ) | 1 | 1.33 |
| CH3(δ1) | 3 | 1.08 |
| CH3(δ2) | 3 | 0.88 |
| N—Me | 3 | 2.57 |
| Amino acid-5 | | |
| CH(α) | 1 | 4.80 |
| CH(β) | 1 | 2.55 |
| CH3(γ1) | 3 | 1.07 |
| CH3(γ2) | 3 | 0.88 |
| NH | 1 | 7.61 |
| Amino acid-6 | | |
| CH(α) | 1 | 5.39 |
| CH(β1) | 1 | 2.34 |
| CH(β2) | 1 | 1.46 |
| CH(γ) | 1 | 2.15 |
| CH3(δ1) | 3 | 1.16 |
| CH3(δ2) | 3 | 1.05 |
| N—Me | 3 | 3.23 |
| Amino acid-7 | | |
| CH(α) | 1 | 4.79 |
| CH3(β) | 3 | 1.66 |
| NH | 1 | 7.99 |
| Amino acid-8 | | |
| CH(α) | 1 | 4.81 |
| CH3(β) | 3 | 1.00 |
| NH | 1 | 7.68 |
| Amino acid-9 | | |
| CH(α) | 1 | 5.87 |
| CH(β1) | 1 | 2.18 |
| CH(β2) | 1 | 1.25 |
| CH(γ) | 1 | 1.25 |
| CH3(δ1) | 3 | 0.91 |
| CH3(δ2) | 3 | 0.83 |
| N—Me | 3 | 2.96 |
| Amino acid-10 | | |
| CH(α) | 1 | 5.34 |
| CH(β1) | 1 | 2.44 |
| CH(β2) | 1 | 1.29 |
| CH(γ) | 1 | 1.78 |
| CH3(δ1) | 3 | 1.15 |
| CH3(δ2) | 3 | 1.15 |
| N—Me | 3 | 2.86 |
| Amino acid-11 | | |
| CH(α) | 1 | 5.16 |
| CH(β) | 1 | 2.26 |
| CH3(γ1) | 3 | 0.90 |
| CH3(γ2) | 3 | 0.63 |
| N—Me | 3 | 2.99 |

³⁾Chemical shifts (ppm) are expressed in the δ scale.
⁴⁾The trans configuration assignment was based on the coupling constant (J_{εζ} = 15.0 Hz).

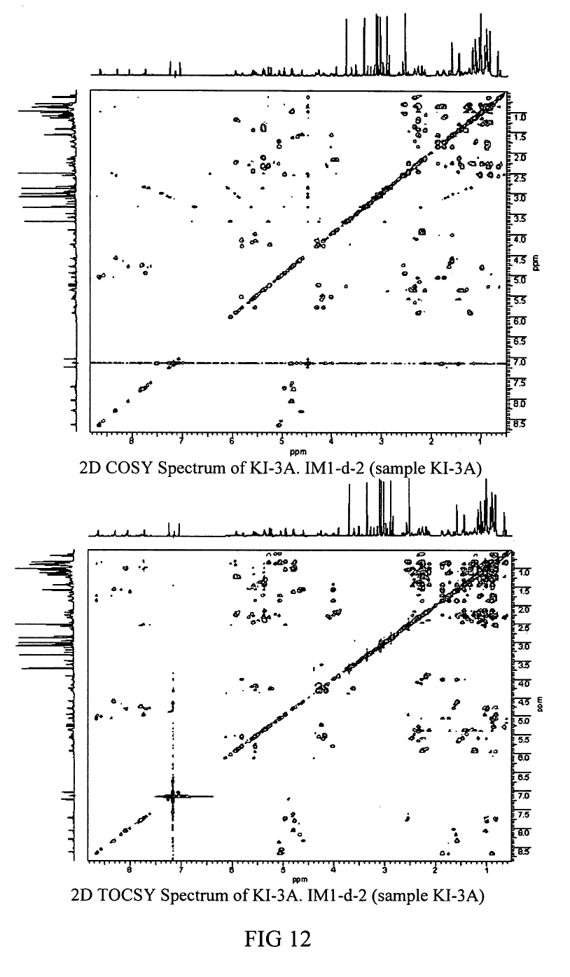

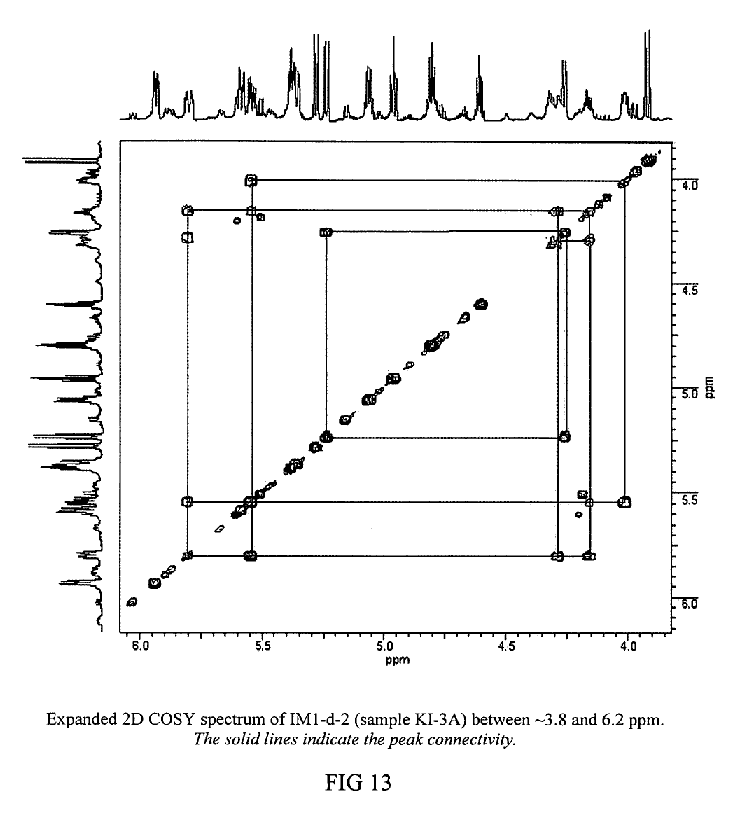

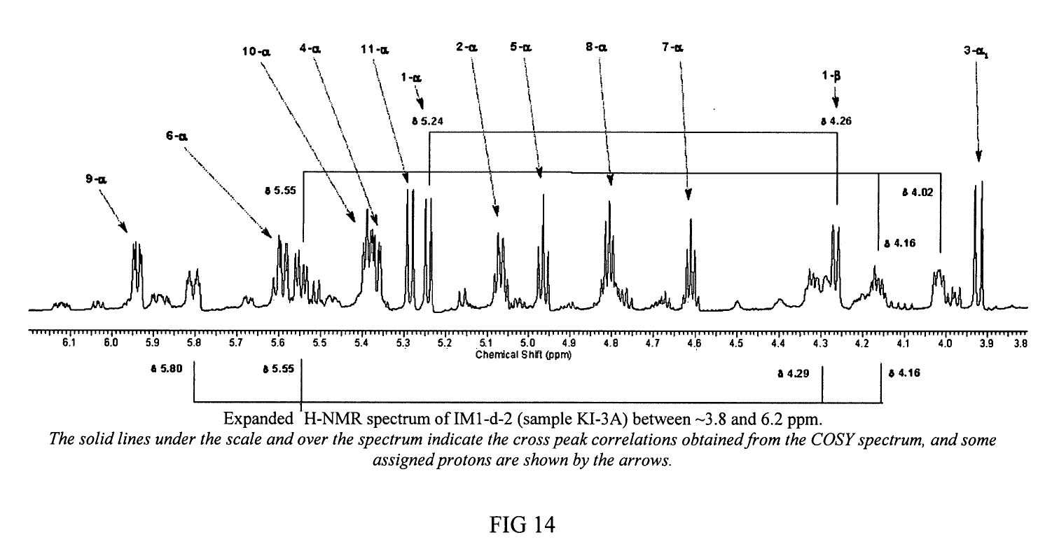

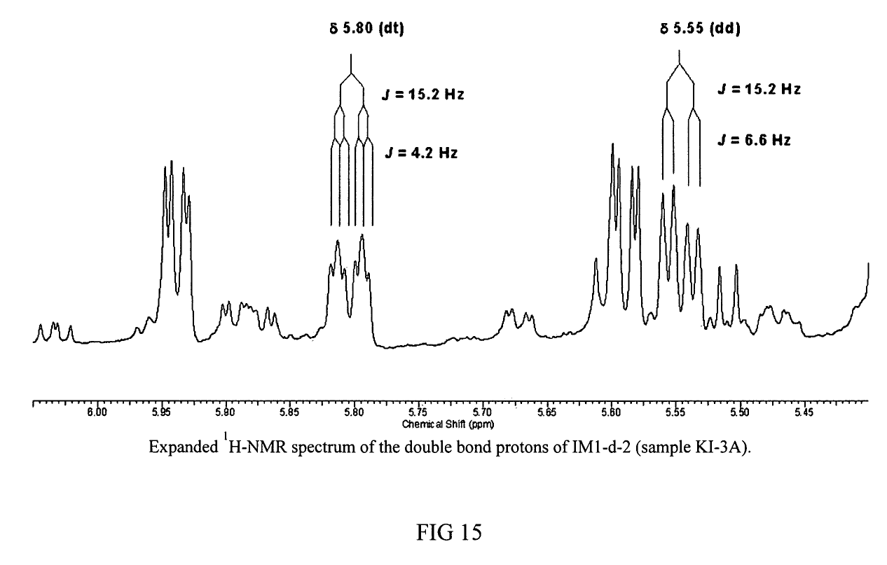

The second diol (sample KI-3A) to be studied, IM1-d-2, was isolated from the chemically transformed ISA247 (cis:trans=5:95) using HPLC purification and analyzed by ¹H-NMR and 2D TOCSY to determine its structure. FIG. 11 shows the ¹H-NMR spectrum of IM1-d-2 compared to the ¹H-NMR spectrum of trans-ISA247. The comparison of these ¹H-NMR spectra demonstrates the loss of the diene protons between δ 6 and 7 ppm, implying a transformation at the side chain of aa-1. The observation of four amide NH doublets and seven N-methyl singlets in the usual regions suggested that the cyclic peptide structure is intact. FIG. 12 shows 2D COSY and TOCSY spectra of the diol. These spectra provide the connectivity of the signals, which enable the straightforward assignment of most of the protons, except those of the aa-1 side chain where the changes were observed. The expansion of the COSY spectrum of the ax proton region between δ 3.8 and 6.2 ppm is shown in FIG. 13, with solid lines indicating the cross peak correlations. This is also demonstrated in the expanded ID ¹H-NMR spectum, shown in FIG. 14, with some additional assignments of the protons.

The COSY spectrum demonstrated that there are four cross-peak correlations in the region:

The signal at δ 5.80 ppm to the ones at δ 5.55, 4.29 and 4.16 ppm.

The signal at δ 5.55 ppm to the ones at δ 4.16 and 4.02 ppm.

The signal of 1-α at d 5.24 ppm to the one of 1-β at δ 4.26 ppm.

The signal at δ 4.29 to the one at δ 4.16.

The signals observed at δ 5.80 and 5.55 ppm are indicative of the olefinic protons (—CH═CH—), and the signals at δ 4.29, 4.16 and 4.02 ppm are of the protons attached to alcoholic carbons (>CH—OH). The coupling relationships between the signals at δ 4.29 and 4.16 ppm, and between these signals and the olefinic proton signal at δ 5.80 ppm, suggested that the signals at δ 4.29 and 4.16 ppm are of the methylene protons of the primary alcohol (HO—CH₂—) attached to the double bond (—C═CH—), resulting in the allylic alcohol structure of (HO—CH₂—CH═CH2). Furthermore, the coupling of the signal at δ 4.02 ppm with the other olefinic proton at δ 5.55 ppm indicated the following overall structure for these protons:

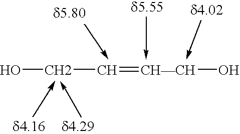

The above structure also agrees with a long range, four bond coupling (⁴J) connectivity observed in the COSY spectrum between the protons at δ 5.55 and δ 4.16 ppm.

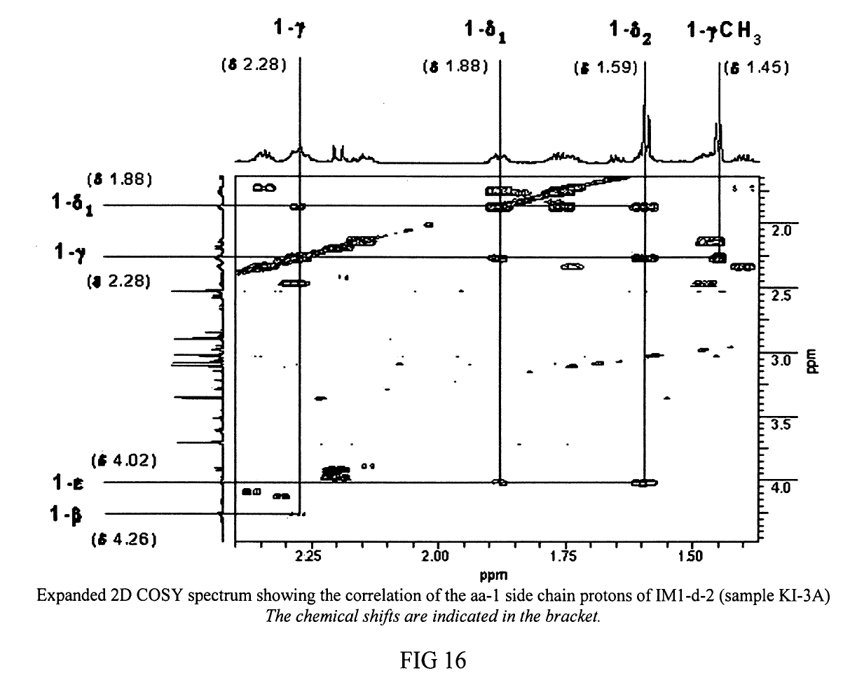

The double bond configuration was assigned as trans from the observed coupling constant of 15.2 Hz, as shown in the expanded 1D spectrum of the double bond protons in FIG. 15. The splitting pattern of the signal (doublet and triplet) at δ 5.80 ppm, due to the coupling with the methylene protons ad δ 4.29 and 4.16 ppm, and the other double bond proton at δ 5.55 ppm, is in agreement with the proposed structure. The slightly broad appearance of the doublet and doublet signal observed for the double bond proton at δ 5.55 ppm could be explained by the existence of ⁴J with the signal at δ 4.16 ppm.

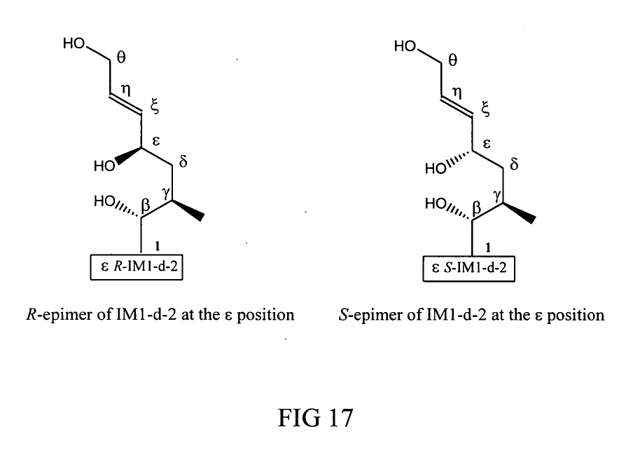

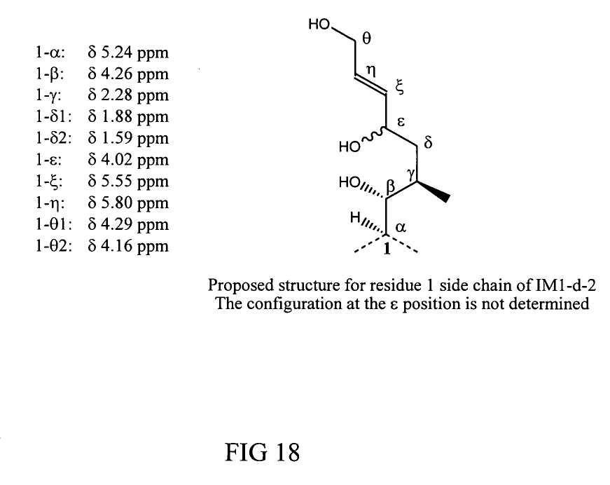

The following signal connectivity of the rest of the aa-1 side chain protons was additionally obtained from the COSY spectrum: 1-β at δ 4.26 ppm to 1-y at 6 2.28 pm; 1-γ at δ 2.28 ppm to 1-δ at δ 1.88 ppm, 1-δ at δ 1.59 ppm and 1-γCH₂ at δ 1.45 ppm; 1-ε at δ 4.02 ppm to 1-δ at δ 1.88 ppm, 1-δ at δ 1.59 ppm. These correlations are demonstrated in FIG. 16 of the partially expanded COSY spectrum. The above spectroscopic findings indicated that the IM1-d-2 structure is as shown in FIG. 17.

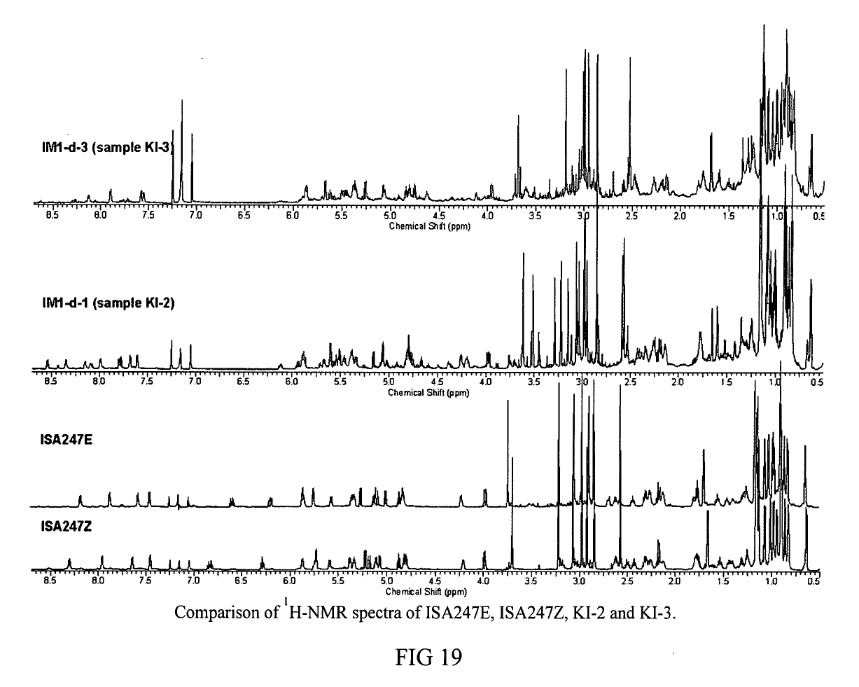

FIG. 17 illustrates the structure of IM1-d-2 in two diastereomeric forms. Those of skill in the art will note that the structure illustrated in FIG. 17 is in the trans double bond configuration at aa-1, and is a double bond migrated product (IM1-d-2) formed by the epoxide (EM1-e-2) ring opening with concerted water attack at the ε position as mechanistically illustrated in FIG. 42. Therefore, the compound can exist in either of the diastereomeric forms shown in FIG. 17. Unlike the IM1-d-1 structure discussed above, however, this compound rendered a relatively clean NMR spectrum. Therefore, it is proposed that the structure is one of the diastereomers shown in FIG. 17. FIG. 18 shows the amino acid-1 structure of IMN1-d-2 with chemical shift assigriment for protons attached to carbons. Compiled spectral data of Ml -d-2, with chemical shift assignments, is presented in Table 2.

TABLE 2
¹H-NMR Chemical shift assignment of IM1-d-2 (KI-3A).^{1),2)}

| Amino acid | Hs | Chemical shift (ppm)³⁾ | Coupling constant (Hz) |
|---|---|---|---|
| Amino acid-1 | | | |
| CH(α) | 1 | 5.24 | d, 10.6 |
| CH(β) | 1 | 4.26 | d, 10.6 |
| CH(γ) | 1 | 2.28 | m |
| γ-CH3 | 3 | 1.45 | d, 7.1 |
| CH(δ1) | 1 | 1.88 | m |
| CH(δ2) | 1 | 1.59 | m |
| CH(ε) | 1 | 4.02 | dd, 10.5 and 6.6 |
| CH(ζ) | 1 | 5.55 | dd, 15.2 and 6.6 |
| CH(η) | 1 | 5.80 | dt, 15.2 and 4.2 |
| CH(θ1) | 1 | 4.29 | m |
| CH(θ2) | 1 | 4.16 | m |
| N—Me | 3 | 3.71 | s |
| Amino acid-2 | | | |
| CH(α) | 1 | 5.06 | m |
| CH(β1) | 1 | 1.88 | m |
| CH(β2) | 1 | 1.76 | m |
| CH3(γ) | 3 | 0.91 | overlap |
| NH | 1 | 8.64 | d, 9.8 |
| Amino acid-3 | | | |
| CH(α1) | 1 | 3.92 | d, 13.4 |
| CH(α2) | 1 | 2.20 | d, 13.4 |
| N—CH3 | 3 | 3.11 | S |
| Amino acid-4 | | | |
| CH(α) | 1 | 5.36 | dd, 11.9 and 3.6 |
| CH(β1) | 1 | 2.15 | m |
| CH(β2) | 1 | 1.47 | m |
| CH(γ) | 1 | 1.32 | m |
| CH3(δ1) | 3 | 1.02 | overlap |
| CH3(δ2) | 3 | 0.90 | overlap |
| N—CH3 | 3 | 2.53 | s |
| Amino acid-5 | | | |
| CH(α) | 1 | 4.96 | t, 9.2 |
| CH(β) | 1 | 2.55 | m |
| CH3(γ1) | 3 | 1.05 | d, 6.5 |
| CH3(γ2) | 3 | 0.84 | d, 6.5 |
| NH | 1 | 7.74 | d, 9.2 |
| Amino acid-6 | | | |
| CH(α) | 1 | 5.59 | dd, 12.2 and 3.8 |
| CH(β1) | 1 | 2.48 | m |
| CH(β2) | 1 | 1.48 | m |
| CH(γ) | 1 | 2.28 | m |
| CH3(δ1) | 3 | 1.18 | d, 6.6 |
| CH3(δ2) | 3 | 1.02 | overlap |
| N—CH3 | 3 | 3.36 | s |
| Amino acid-7 | | | |
| CH(α) | 1 | 4.61 | m |
| CH3(β) | 3 | 1.59 | d, 7.1 |
| NH | 1 | 8.31 | d, 6.8 |
| Amino acid-8 | | | |
| CH(α) | 1 | 4.80 | m |
| CH3(β) | 3 | 1.02 | overlap |
| NH | 1 | 8.07 | d, 7.9 |
| Amino acid-9 | | | |
| CH(α) | 1 | 5.93 | dd, 11.3 and 3.8 |
| CH(β1) | 1 | 2.27 | m |
| CH(β2) | 1 | 1.23 | m |
| CH(γ) | 1 | 1.24 | m |
| CH3(δ1) | 3 | 0.94 | d, 6.5 |
| CH3(δ2) | 3 | 0.84 | d, 6.5 |
| N—CH3 | 3 | 3.09 | S |
| Amino acid-10 | | | |
| CH(α) | 1 | 5.38 | t, 7.0 |
| CH(β1) | 1 | 2.35 | m |
| CH(β2) | 1 | 1.41 | m |
| CH(γ) | 1 | 1.74 | m |
| CH3(δ1) | 3 | 1.13 | d, 6.4 |
| CH3(δ2) | 3 | 1.12 | d, 6.4 |
| N—CH3 | 3 | 2.90 | s |
| Amino acid-11 | | | |
| CH(α) | 1 | 5.28 | d, 11.0 |
| CH(β) | 1 | 2.35 | m |
| CH3(γ1) | 3 | 0.88 | d, 6.5 |
| CH3(γ2) | 3 | 0.67 | d, 6.5 |
| N—CH3 | 3 | 3.03 | s |

¹⁾The chemical shifts for N-methyl signals were assigned based on the four bond coupling (⁴J) and the five bond coupling (⁵J) correlations obtained from COSY and TOCSY spectra.
²⁾The trans configuration was assigned for the double bond at aa-1, based upon the proton coupling constant of 15.2 Hz between H(ζ) and H (η) of aa-1.
³⁾Chemical shifts (ppm) are expressed in the δ scale.

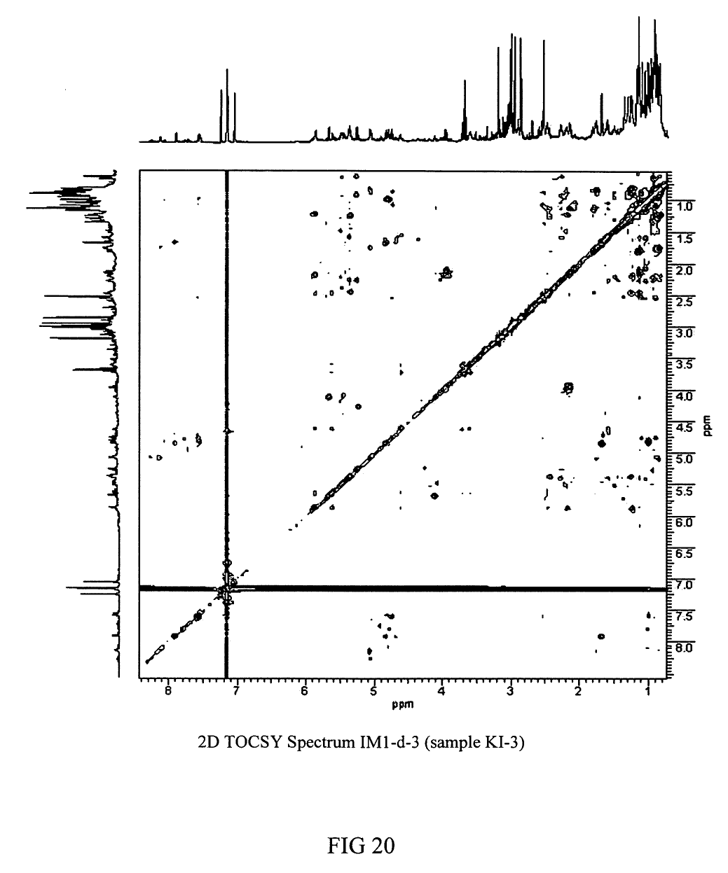

The third diol metabolite, IM1-d-3 (sample KI-3), was obtained from the microorganism biotransfomation of ISA247 (cis:trans=50:50) with HPLC purification. The ¹H-NMR spectra of the IM1-d-3 compound (sample KI-3) compared to the IM1-d-1 compound along with trans-ISA247 (E-ISA247) and cis-ISA247 (Z-ISA247) is shown in FIG. 19. Comparison of NMR spectra shown in FIG. 19 revealed changes in the IM1-d-3 (sample KI-3) ¹H-NMR spectrum at the diene region between δ 6 and 7 ppm, indicative of the aa-1 side chain transformation. The mass study information also indicated the changes at the amino acid-1 side chain with formation of a diol. These changes are similar to those obtained from the IM1-d-1 metabolite, though the spectral comparison indicated that IM1-d-3 and IM1-d-1 are structurally different.

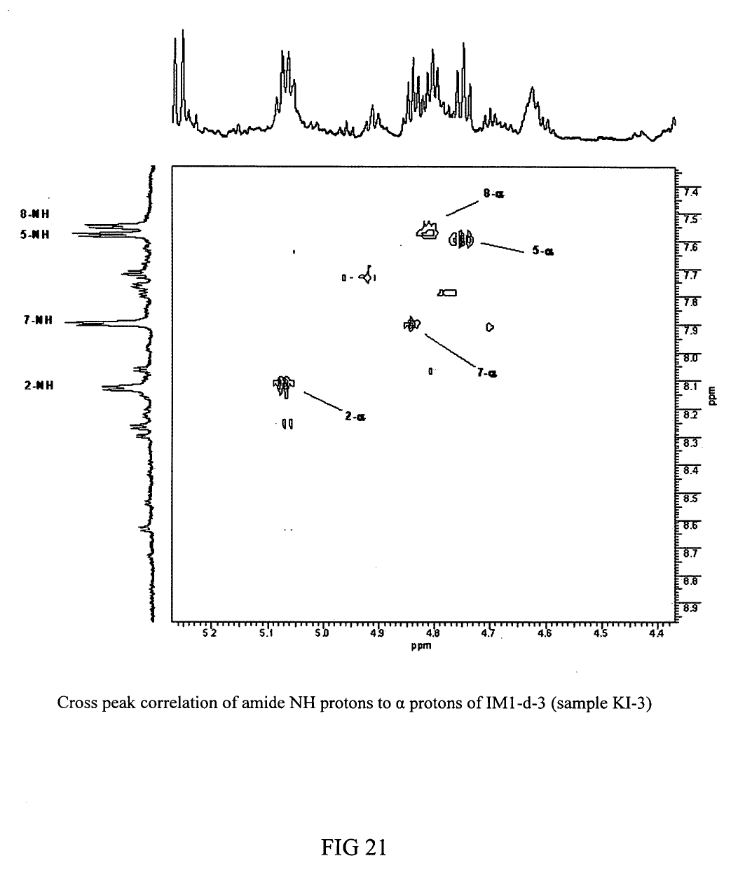

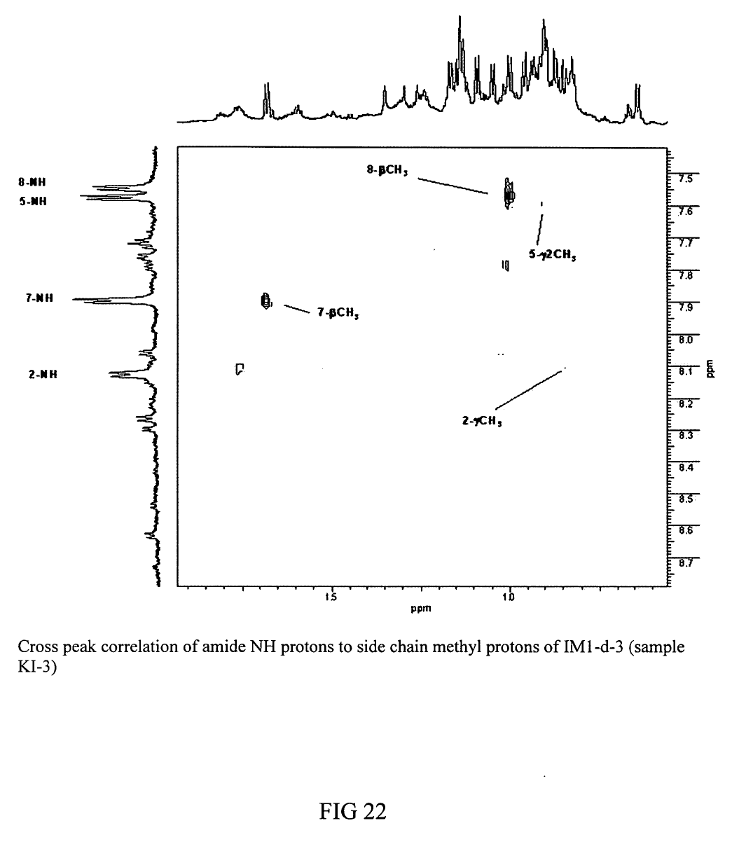

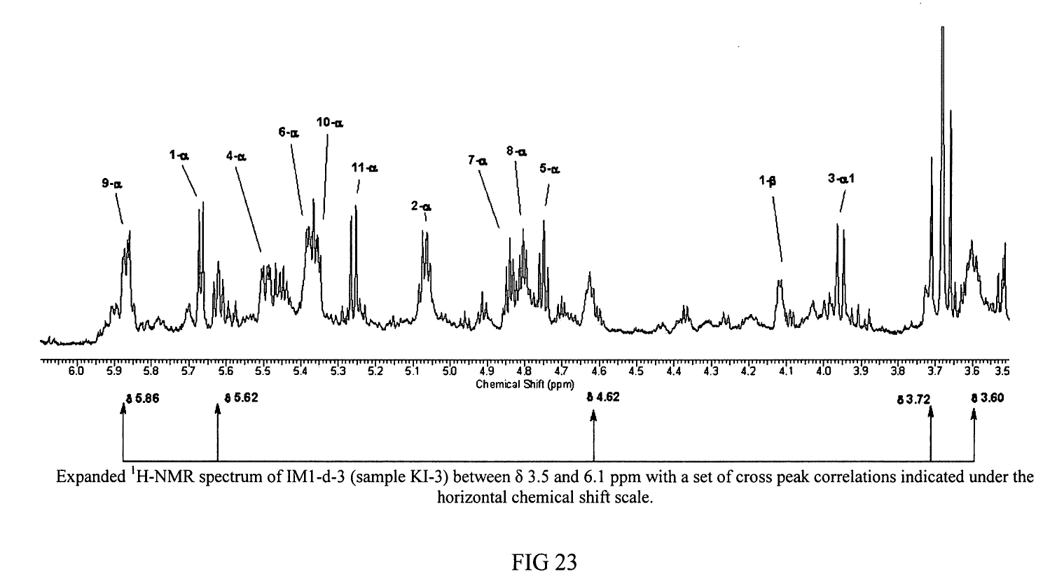

The amide NH proton and the N-methyl proton region of the spectrum indicated that sample KI-3 is a mixture of one major and several minor contaminations. The NMR analysis, therefore, concentrated on the major product of KI-3 (IM1-d-3), mainly by cross-peak correlations obtained from the 2D TOCSY spectrum (See FIG. 20). The 2D TOCSY spectrum at the amide NH proton region showed that the major amide NH protons are of aa-2, aa-5, aa-7 and aa-8, as observed typically in ISA247 and its metabolites, and are identifiable by observing 2D correlation to characteristic signals, such as 2-γCH₃, 7-βCH₃ and 8-βCH₃. The expanded TOCSY spectra of the amide proton correlations are shown in FIGS. 21 and 22 with some shift assignments. Chemical shifts of most of amino acid side chain protons of the major metabolite were similarly assigned based upon the TOCSY spectrum correlation. The existence of four amide protons and seven N-methyl group singlets, and similar signal connectivity patterns to those of ISA247 and other metabolites, indicated the cyclic ring structure of the parent ISA247 is intact.

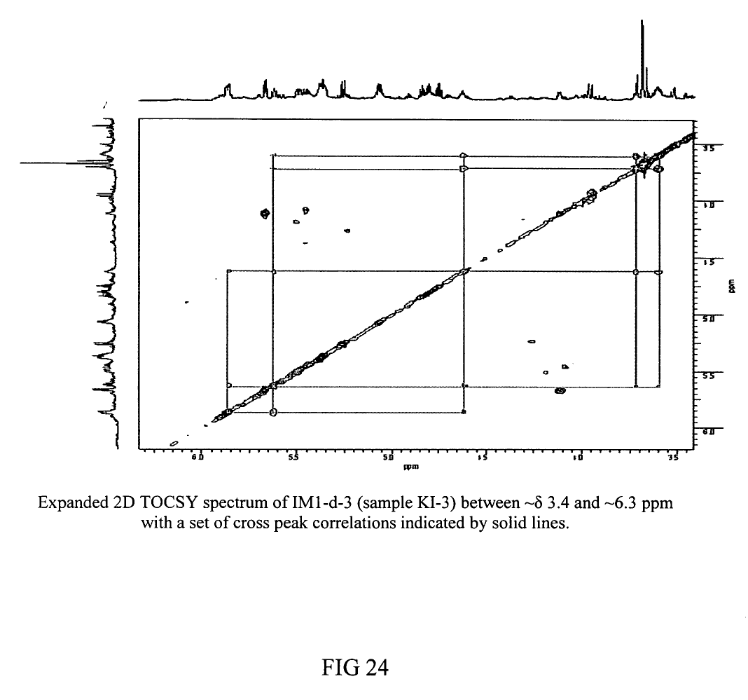

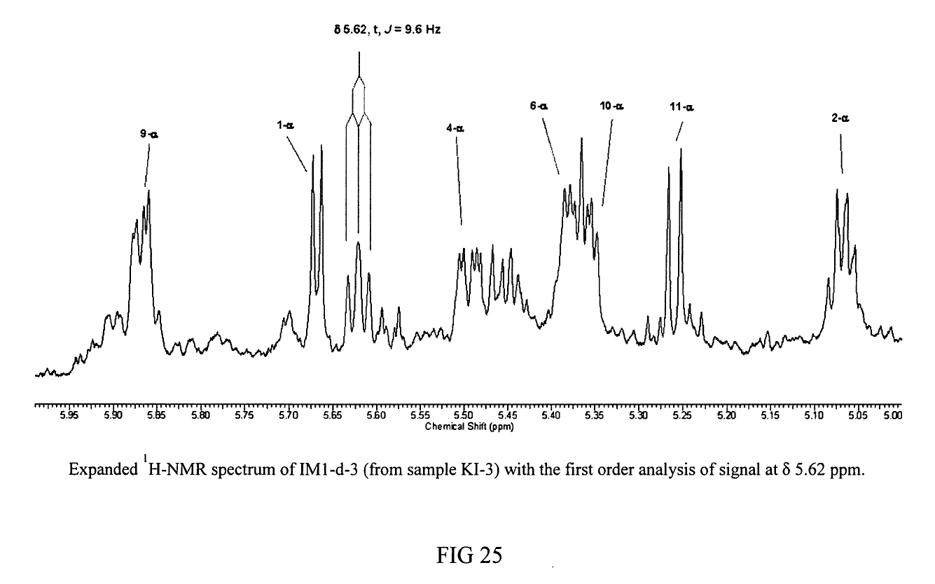

Close examination of the a proton region of the 2D TOCSY spectrum demonstrated a set of correlation signals at δ 5.86-5.62-4.62-3.72-3.60 ppm (see FIG. 24.), presumably of the amino acid-1 side chain protons. This was also shown in the expanded ID spectrum at this region (see FIG. 23). The proton signals at δ 5.86 (overlap with 9-α) and 5.62 ppm are clearly indicative of olefinic protons (—CH═CH—); the one at δ 5.62 of a secondary alcohol proton (>CH-OH); and those at 8 3.72 and 3.60 ppm of primary alcohol protons (—CH2—OH). These proton correlations indicated by solid lines in FIG. 24 are similar to those observed for metabolite IM1-d-1 (KI-2 or KI-2A), though the proton at δ 5.62 ppm of IM1-d-3 is more downfield shifted. Thus, the NMR spectrum and mass information on metabolite IM1-d-3 is in agreement with a diol formation at amino acid-1.

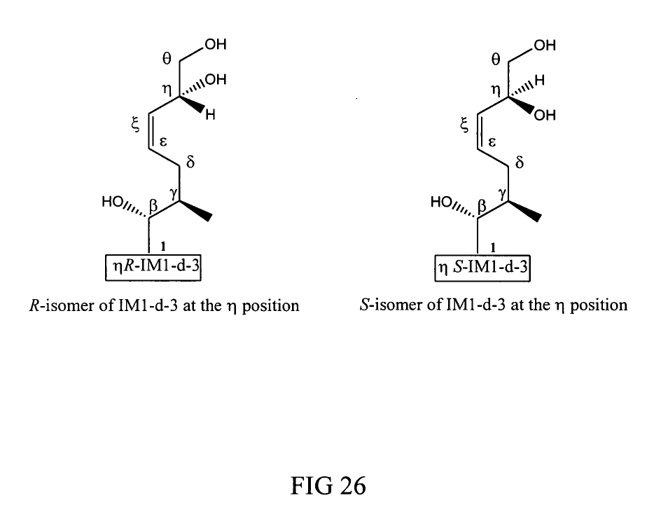

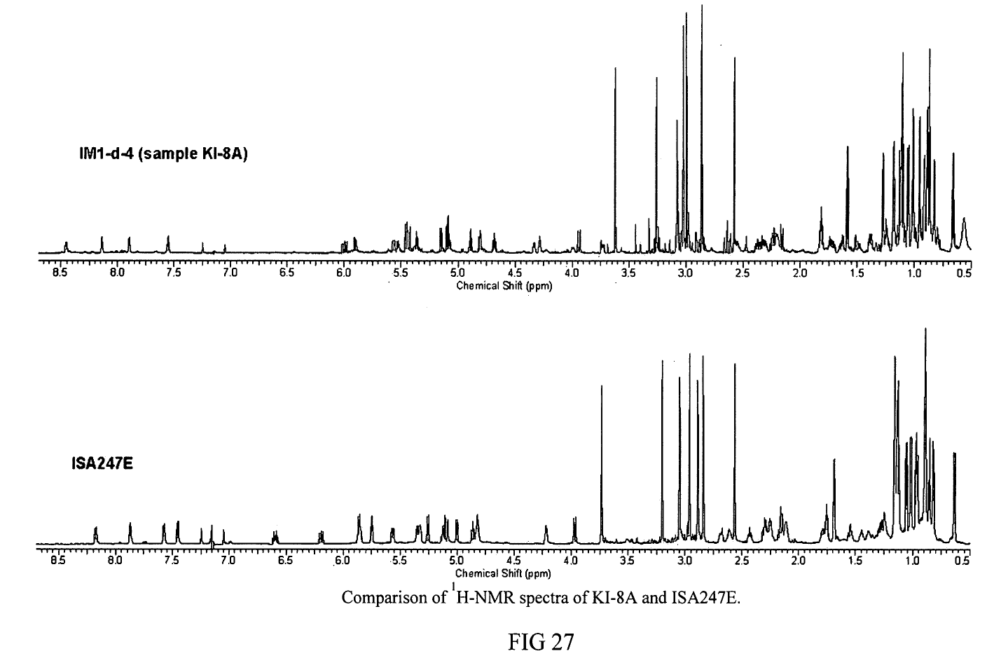

The first order analysis of the signal at δ 5.62 ppm (see FIG. 25) showed the triplet with a coupling constant of 9.6 Hz, indicating the double bond of the cis configuration, even though the assumed counter proton of the double bond is overlapping with 9-α and unavailable for the analysis. The chemical shifts at δ 3.72 and 3.60 ppm suggested that the structural group (—CH₂—OH) is not directly bound to the double bond. Therefore, based upon these observations, it is proposed that the structures in FIG. 26 are the major product in sample KI-3, metabolite IM1-d-3. The IM1-d-3 diol can exist as two diastereomers, as shown in FIG. 26, differing at the chiral η postion of aa-1. The stereochemistry of the η position of the structure seen in FIG. 26 is not determined. However, when another sample KI-4A which was a chemically synthesized sample made from ISA247 in a cis:trans ratio of 1:1 was studied, the NMR analysis of sample KI-4A (not shown) showed that the major component of the KI-4A sample is a diasteroisomer of the major component of the KI-3 sample, differing from each other in the stereochemistry at the η position of aa-1. The KI-4A sample displayed distinct NMR spectra from those presented in FIGS. 19-25 for the KI-3 sample. While it was possible to identify these structures, and it was possible to isolate and study each diastereomer, it was not possible to assign one of the diastereomeric structures definitively to one sample or the other. It was not possible to determine which of the R- or S-isomer at the η position of aa-1 belongs to the KI-4A or KI-3 samples.

FIG. 26 illustrates that IM1-d-3 is a diol in the cis configuration with a chiral center at the η carbon of aa-1, therefore, existing as two diastereomers (KI-3 and KI-4A). IM1-d-3 is the same diol as IM-1-d-1, except that the double bond in IM-1-d-3 is in the cis conformation and that in IM1-d-1 is in the trans conformation. This difference separates the IM1-d-1 and IM1-d-3 metabolites in the HPLC scan shown in FIG. 6. Chemical shift assignments for IM1-d-3 are presented in Table 3.

TABLE 3
¹H-NMR Chemical shift assignment of IM1-d-3 (KI-3).^{1),2)}

| | | Chemical shift |
|---|---|---|
| Amino acid | Hs | (ppm)³⁾ |
| Amino acid-1 | | |
| CH(α) | 1 | 5.68 |
| CH(β) | 1 | 4.12 |
| CH(γ) | 1 | 2.14 |
| γ-CH3 | 3 | 1.14 |
| CH(δ1) | 1 | 2.48 |
| CH(δ2) | 1 | ˜2.14 |
| CH(ε) | 1 | 5.86 |
| CH(ζ)⁴⁾ | 1 | 5.62 |
| CH(η) | 1 | 4.62 |
| CH(θ1) | 1 | 3.72 |
| CH(θ2) | 1 | 3.60 |
| N—Me | 3 | 3.68 |
| Amino acid-2 | | |
| CH(α) | 1 | 5.07 |
| CH(β1) | 1 | 1.76 |
| CH(β2) | 1 | 1.76 |
| CH3(γ) | 3 | 0.85 |
| NH | 1 | 8.12 |
| Amino acid-3 | | |
| CH(α1) | 1 | 3.95 |
| CH(α2) | 1 | 2.13 |
| N—CH3 | 3 | 3.01 |
| Amino acid-4 | | |
| CH(α) | 1 | 5.50 |
| CH(β1) | 1 | 2.21 |
| CH(β2) | 1 | 1.49 |
| CH(γ) | | 1.31 |
| CH3(δ1) | 3 | 0.96 |
| CH3(δ2) | 3 | 0.88 |
| N—CH3 | 3 | 2.52 |
| Amino acid-5 | | |
| CH(α) | 1 | 4.75 |
| CH(β) | 1 | 2.53 |
| CH3(γ1) | 3 | 1.10 |
| CH3(γ2) | 3 | 0.90 |
| NH | 1 | 7.58 |
| Amino acid-6 | | |
| CH(α) | 1 | 5.38 |
| CH(β1) | 1 | 2.27 |
| CH(β2) | 1 | 1.60 |
| CH(γ) | 1 | 2.08 |
| CH3(δ1) | 3 | 1.14 |
| CH3(δ2) | 3 | 1.05 |
| N—CH3 | 3 | 3.19 |
| Amino acid-7 | | |
| CH(α) | 1 | 4.84 |
| CH3(β) | 3 | 1.69 |
| NH | 1 | 7.90 |
| Amino acid-8 | | |
| CH(α) | 1 | 4.80 |
| CH3(β) | 3 | 1.01 |
| NH | 1 | 7.55 |
| Amino acid-9 | | |
| CH(β1) | 1 | 2.18 |
| CH(β2) | 1 | 1.24 |
| CH(γ) | 1 | 1.24 |
| CH3(δ1) | 3 | 0.91 |
| CH3(δ2) | 3 | 0.82 |
| N—CH3 | 3 | 2.95 |
| Amino acid-10 | | |
| CH(α) | 1 | 5.36 |
| CH(β1) | 1 | 2.45 |
| CH(β2) | 1 | 1.27 |
| CH(γ) | 1 | 1.80 |
| CH3(δ1) | 3 | 1.17 |
| CH3(δ2) | 3 | 1.14 |
| N—CH3 | 3 | 2.86 |
| Amino acid-11 | | |
| CH(α) | 1 | 5.26 |
| CH(β) | 1 | 2.28 |
| CH3(γ1) | 3 | 0.94 |
| CH3(γ2) | 3 | 0.64 |
| N—CH3 | 3 | 2.99 |

¹⁾The assignment was made primarily based upon TOCSY spectrum.
²⁾The N-methyl signals were tentatively assigned, so some of them may be interchangeable.
³⁾Chemical shifts (ppm) are expressed in the δ scale.
⁴⁾Cis configuration was assigned for the double bond at aa-1, based upon the observed triplet for this proton with the coupling constant of 9.6 Hz.

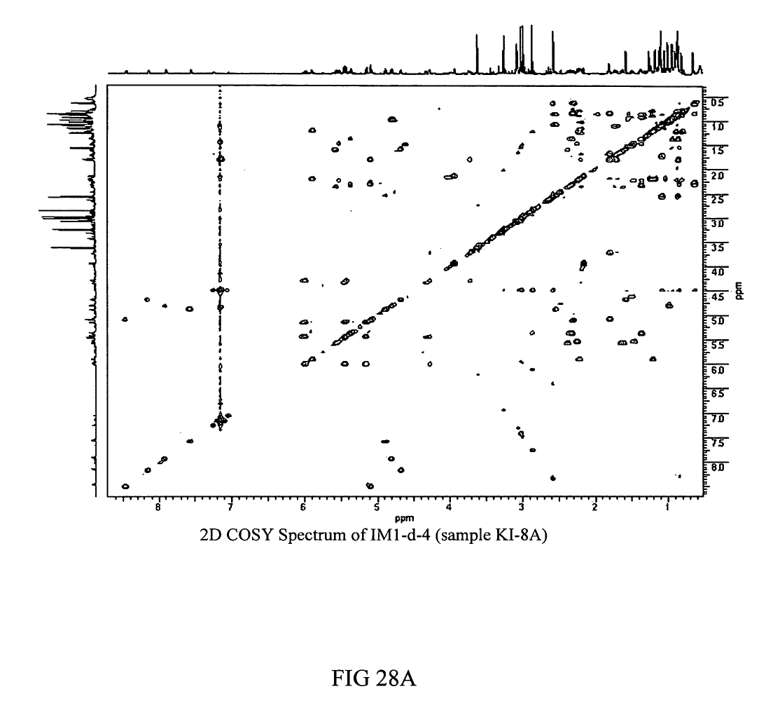

The fourth diol to be isolated was IM1-d-4 (sample KI-8A). Sample KI-8A with minor contaminations was obtained from the chemical transformation of ISA247 (cis trans=5:95), in addition to samples KI-2A (IM1-d-1) and KI-3A(IM1-d-2). The NMR analysis of HPLC isolated IM1-d-4 (sample KI-8A) showed a loss of the diene protons between δ 6 and 7 ppm, implying a transformation at the side chain of aa-1 as expected. The observation of four amide NH doublets and seven N-methyl singlets in the usual regions suggested that the cyclic peptide structure is intact. FIG. 27 is a comparison of ¹H-NMR spectra of 1M1-d-4 (sample KI-8A) and trans ISA247 or E-ISA247.

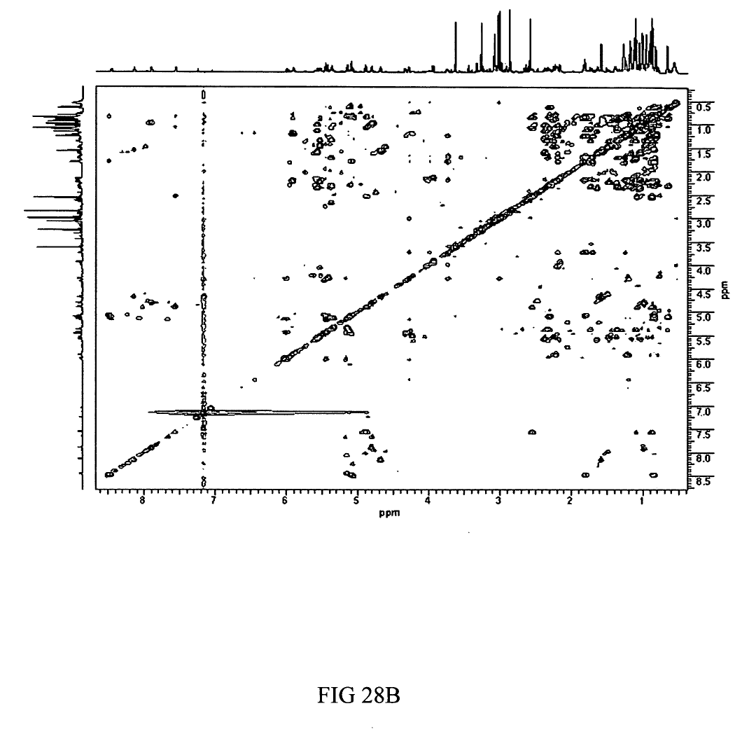

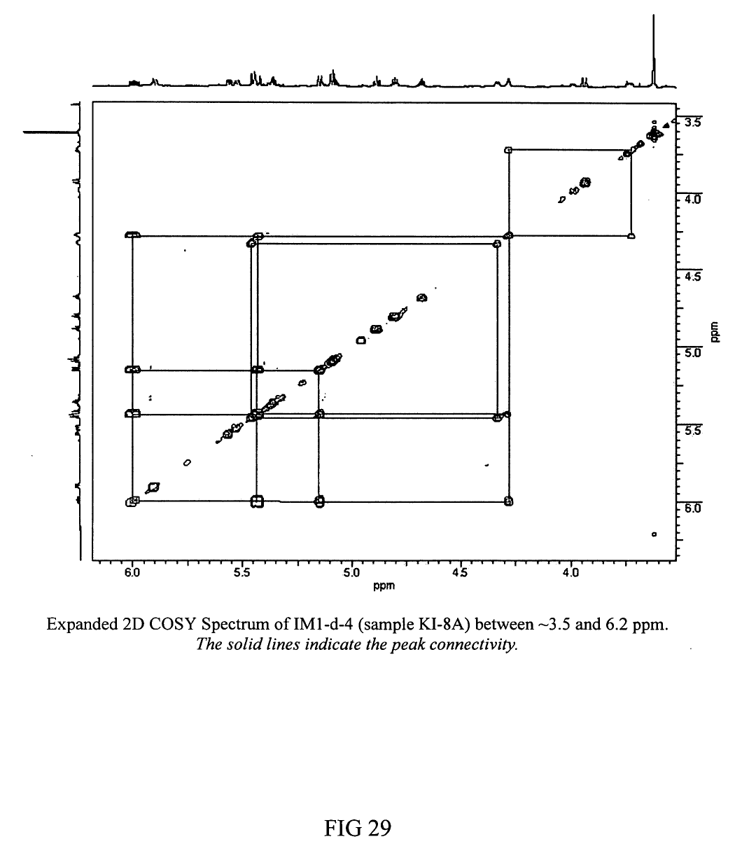

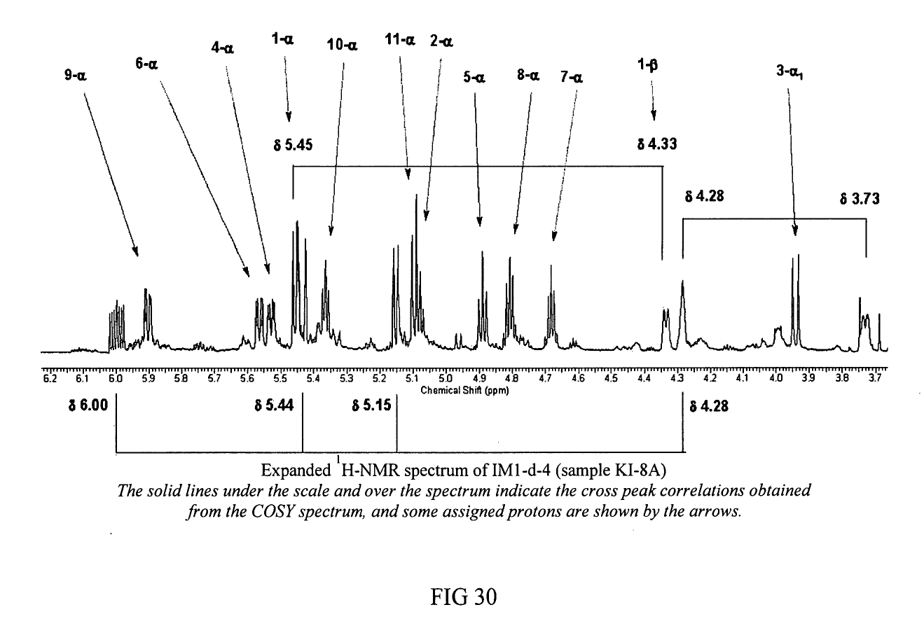

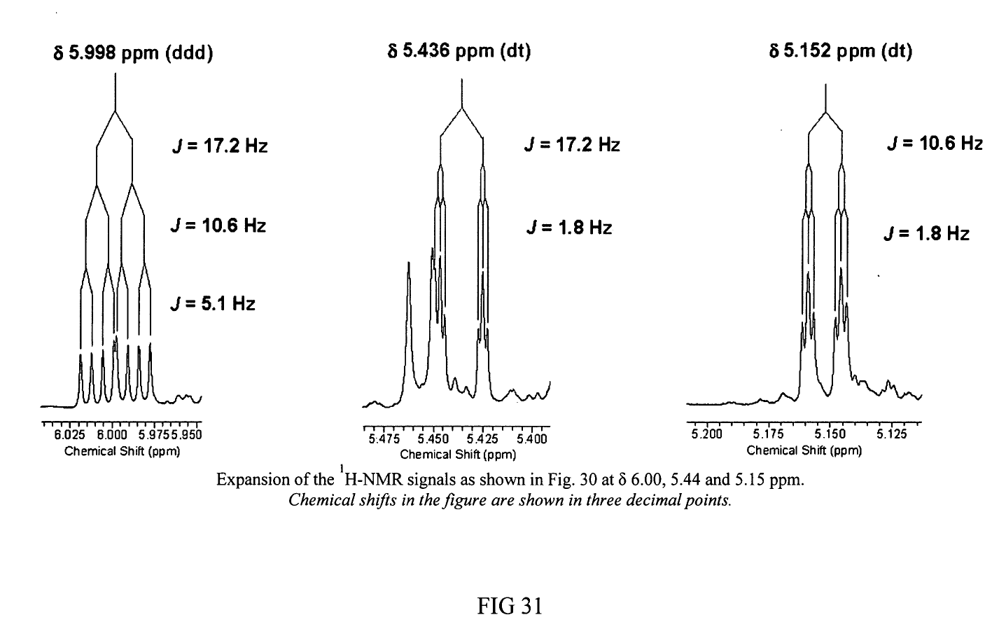

IM1-d-4 appears to be the major component of sample KI-8, though minor contaminants are visible in the spectrum. Again, an analysis of the 2D COSY and TOCSY spectra, shown in FIGS. 28A and 28B, provided the shift assignment for most of the protons, except for a few peaks observed at the a proton region of the spectrum due to the transformation of the aa-1 side chain. This comparison demonstrates the loss of the diene protons between δ-6 and 7 ppm, implying a transformation at the side chain of aa-1. The expanded 2D COSY spectrum in the ax proton region, as shown in FIG. 29, demonstrates the cross-peak connectivity of 1-α and 1-β and unassigned protons—presumably the aa-1 side chain protons. The expanded ID spectrum of the region is shown in FIG. 30 with some peak assignments and peak correlations obtained from the COSY spectrum. The connectivity of signals at δ 6.00 (ddd), 5.44 (dt), 5.15 (dt), 4.28 (m) and 3.73 ppm (m), each integrated as one proton, indicated that these protons are structurally related, such as the side chain protons of aa-1. Of these, the signals at δ 6.00 (ddd), 5.44 (dt) and 5.15 ppm (dt) which displayed discemable splitting patterns, were expanded, as shown in FIG. 31.

The chemical shift of δ 6.00 ppm implied that this proton is an olefinic proton coupled to the protons at δ 5.44 ppm with J=17.2 Hz and at δ 5.15 ppm with J=10.6 Hz, both of which are coupled to each other with a coupling constant of 1.8 Hz and are indicative of terminal double bond geminal protons, as seen in the spectra of E-ISA247 and Z. The observation of four amide NH doublets and seven N-methyl singlets in the usual regions suggested that the cyclic peptide structure is intact.

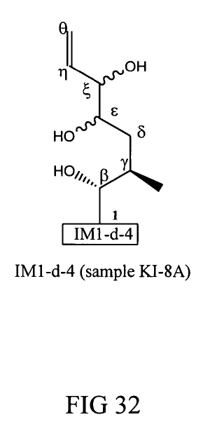

The overall structure of IM1-d-4 is illustrated in FIG. 32. Note that the ε and ζ carbons of aa-1 are both chiral centers. Therefore, the structure shown in FIG. 32 can take four configurations. The exact configuration of the compound with the structure shown in FIG. 32 was not determined. However, it is suggested that the minor contaminants may contain stereoisomers at the ε and ζ carbons of the aa-1 side chain of the major compound.

Chemical shift assignments for IM1-d-4 are presented in Table 4.

TABLE 4
¹H-NMR Chemical shift assignment of IM1-d-4 (KI-8A).¹⁾

| Amino acid | Hs | Chemical shift (ppm)2⁾ | Coupling constant (Hz) |
|---|---|---|---|
| Amino acid-1 | | | |
| CH(α) | 1 | 5.46 | d, 9.5 |
| CH(β) | 1 | 4.33 | d, 9.5 |
| CH(γ) | 1 | 2.20 | m |
| γ-CH3 | 3 | 1.27 | d, 6.9 |
| CH(δ1) | 1 | 1.82 | m |
| CH(δ2) | 1 | 1.70 | m |
| CH(ε) | 1 | 3.73 | m |
| CH(ζ) | 1 | 4.28 | m |
| CH(η) | 1 | 6.00 | ddd, 17.2, 10.6 and 5.1 |
| CH(θ1) | 1 | 5.15 | dt, 10.6 and 1.8 |
| CH(θ2) | 1 | 5.44 | dt, 17.2 and 1.8 |
| N—Me | 3 | 3.63 | s |
| Amino acid-2 | | | |
| CH(α) | 1 | 5.10 | m |
| CH(β1) | 1 | 1.81 | m |
| CH(β2) | 1 | 1.81 | m |
| CH3(γ) | 3 | 0.86 | overlap |
| NH | 1 | 8.45 | d, 9.6 |
| Amino acid-3 | | | |
| CH(α1) | 1 | 3.93 | d, 13.8 |
| CH(α2) | 1 | 2.15 | d, 13.8 |
| N—CH3 | 3 | 3.08 | s |
| Amino acid-4 | | | |
| CH(α) | 1 | 5.52 | dd, 11.2 and 4.0 |
| CH(β1) | 1 | 2.25 | m |
| CH(β2) | 1 | 1.48 | m |
| CH(γ) | 1 | 1.38 | m |
| CH3(δ1) | 3 | 0.96 | d, 6.6 |
| CH3(δ2) | 3 | 0.88 | d, 6.6 |
| N—CH3 | 3 | 2.58 | s |
| Amino acid-5 | | | |
| CH(α) | 1 | 4.89 | t, 9.1 |
| CH(β) | 1 | 2.55 | m |
| CH3(γ1) | 3 | 1.10 | d, 6.6 |
| CH3(γ2) | 3 | 0.88 | d, 6.6 |
| NH | 1 | 7.55 | d, 9.1 |
| Amino acid-6 | | | |
| CH(α) | 1 | 5.56 | dd, 11.6 and 4.1 |
| CH(β1) | 1 | 2.37 | m |
| CH(β2) | 1 | 1.64 | m |
| CH(γ) | 1 | 2.21 | m |
| CH3(δ1) | 3 | 1.18 | d, 6.6 |
| CH3(δ2) | 3 | 1.05 | d, 6.6 |
| N—CH3 | 3 | 3.26 | s |
| Amino acid-7 | | | |
| CH(α) | 1 | 4.69 | m |
| CH3(β) | 3 | 1.58 | d, 7.1 |
| NH | 1 | 8.14 | d, 7.2 |
| Amino acid-8 | | | |
| CH(α) | 1 | 4.82 | m |
| CH3(β) | 3 | 1.01 | d, 6.9 |
| NH | 1 | 7.89 | d, 7.9 |
| Amino acid-9 | | | |
| CH(α) | 1 | 5.90 | dd, 11.9 and 3.5 |
| CH(β1) | 1 | 2.23 | m |
| CH(β2) | 1 | 1.23 | m |
| CH(γ) | 1 | 1.23 | m |
| CH3(δ1) | 3 | 0.91 | d, 6.2 |
| CH3(δ2) | 3 | 0.82 | d, 6.2 |
| N—CH3 | 3 | 3.03 | s |
| Amino acid-10 | | | |
| CH(α) | 1 | 5.36 | t, 7.0 |
| CH(β1) | 1 | 2.35 | m |
| CH(β2) | 1 | 1.39 | m |
| CH(γ) | 1 | 1.73 | m |
| CH3(δ1) | 3 | 1.12 | d, 6.7 |
| CH3(δ2) | 3 | 1.11 | d, 6.7 |
| N—CH3 | 3 | 2.86 | s |
| Amino acid-11 | | | |
| CH(α) | 1 | 5.09 | d, 10.8 |
| CH(β) | 1 | 2.31 | m |
| CH3(γ1) | 3 | 0.86 | d, 6.5 |
| CH3(γ2) | 3 | 0.66 | d, 6.5 |
| N—CH3 | 3 | 3.00 | s |

¹⁾The chemical shifts for N-methyl signals were assigned based on the four bond coupling (⁴J) And the five bond coupling (⁵J) correlations obtained from COSY and TOCSY spectra.
2⁾Chemical shifts (ppm) are expressed in the δ scale.

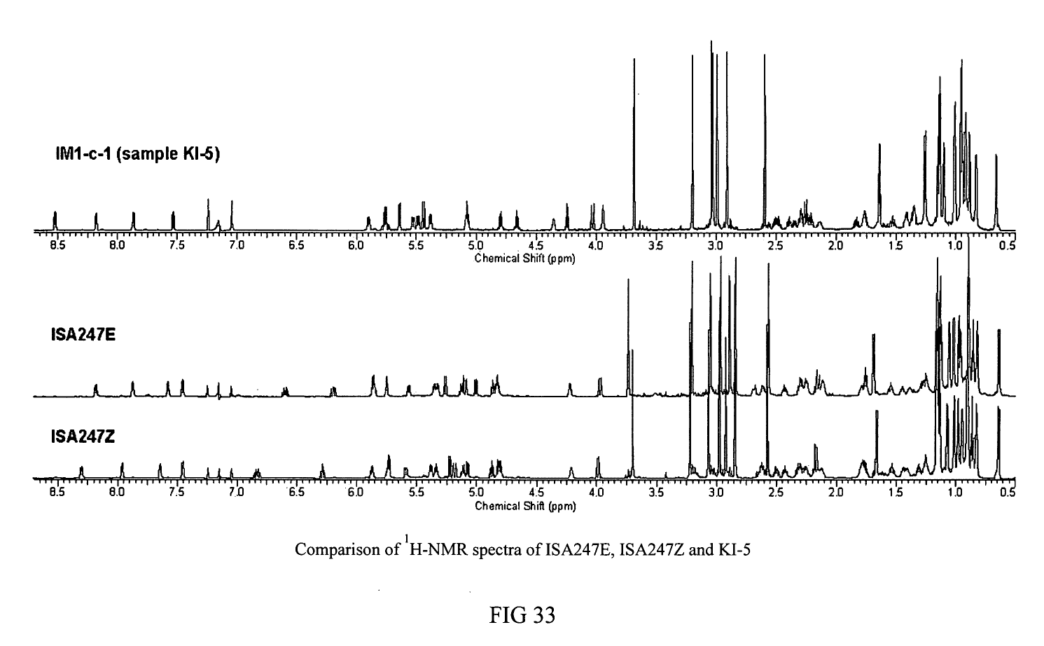

A cyclized metabolite, IM1-c-1 (sample KI-5), was obtained from the biotransformation of ISA247 (cis:trans=1:1) with HPLC purification. The ¹H-NMR of the metabolite (sample KI-5) is shown in FIG. 33 compared to the ¹H-NMR spectra of E-ISA247 and Z-ISA247. The NMR analysis revealed that the sample contained a single compound, and not a diastereoisomeric mixture. This is evident from the amide NH and N-methyl proton region of the spectrum: four amide NH protons and seven N-methyl singlet signals. As seen in FIG. 33, the major change in the spectrum from the ISA compounds to the metabolite is the loss of the protons in the diene region (δ 6-7 ppm).

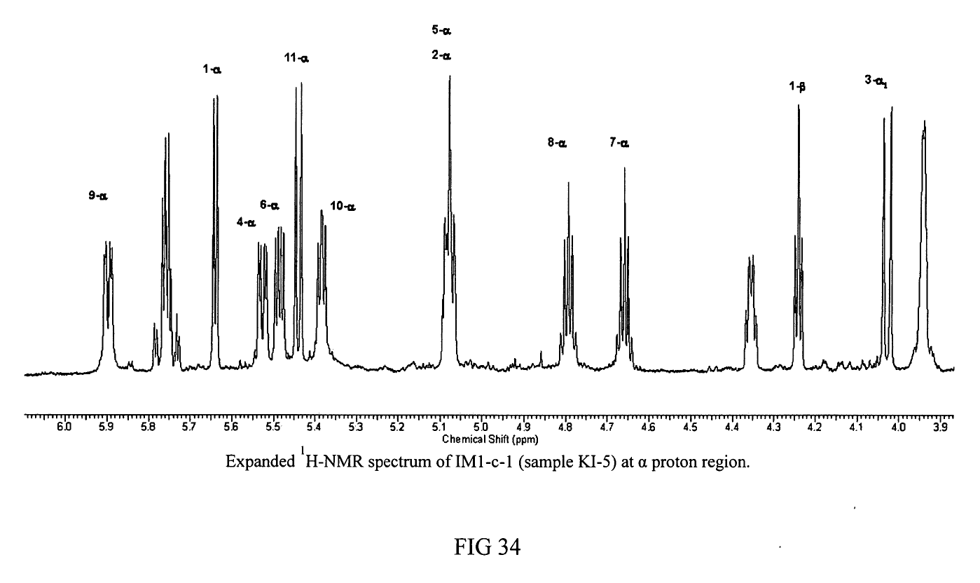

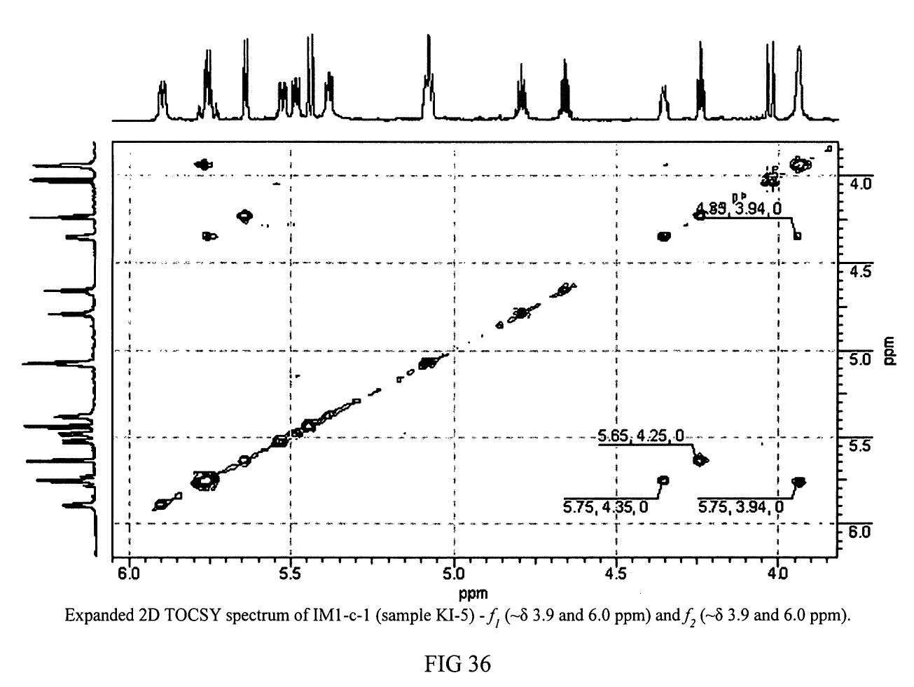

These olefinic proton signals are characteristic for the ISA247 molecule, arising from the amino acid-1 side chain, and are normally observed between δ 6 and 7 ppm. The absence of these peaks from the region, therefore, indicated the structural transformation at the amino acid-1 side chain. The expanded spectrum of the metabolite at the amino acid a proton region between δ 3.9 and 6.0 ppm is shown in FIG. 34, with shift assignments for certain a protons and I-D proton, as indicated on top of the peaks. These assignments and others were obtained from the 2D spectrum, as shown in FIG. 36, and all other amino acid side chain and N-methyl protons are accounted for, except for the amino acid-1 side chain protons. Therefore, it was concluded that the unassigned signals at δ 5.75 (corresponding to 1H by integration), δ 4.35 (1H) and δ 3.94 (2 Hs) in FIG. 34, are of the amino acid-1 side chain protons.

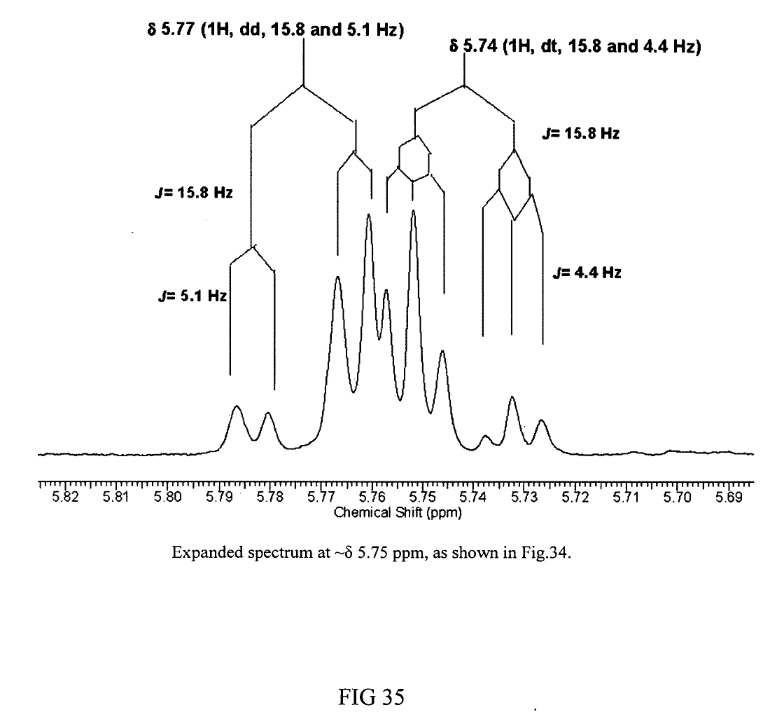

The close examination of the peak at δ 5.75 ppm revealed that it was comprised of two protons, resonating at δ 5.77 and 5.74 ppm, which are also coupled to each other, as shown in FIG. 35. The chemical shifts and the coupling constant (15.8 Hz) suggested the olefinic (—CH═CH—) group having trans configuration. The 2D spectrum of FIG. 36 showed the signals at δ5.75 ppm correlate with the peaks at δ3.94 and 4.35 ppm, both of which in turn show the signal connectivity. Furthermore, the multiplet signal at δ3.94 ppm accounted for two protons, and the one at δ 4.35 for one. Therefore, the structural group (—CH—CH═CH═CH2-) was proposed to explain the signal connectivity of these peaks (δ 5.77, 5.74, 4.35 and 3.9 ppm).

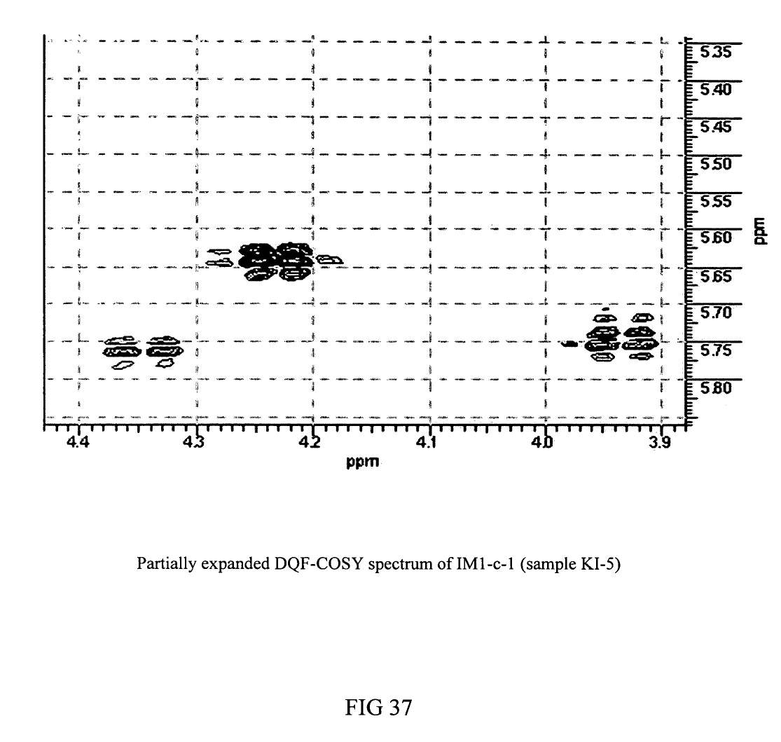

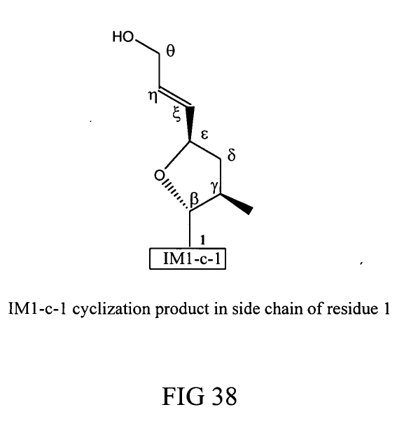

The chemical shifts of the signals at δ 4.35 and 3.94 ppm further suggested that these protons are attached to the carbons next to oxygen atoms having the structure: (—O—CH—CH═CH—CH₂—O—). The expanded DQF-COSY spectrum shown in FIG. 37 revealed that the signal at δ 5.77 ppm is coupled to the one at δ 4.35 ppm, and the signal at δ 5.74 ppm to the one at δ 3.94 ppm. The NMR analysis combined with the mass information clearly pointed to the structure for IM1-c-1, as shown in FIG. 38.

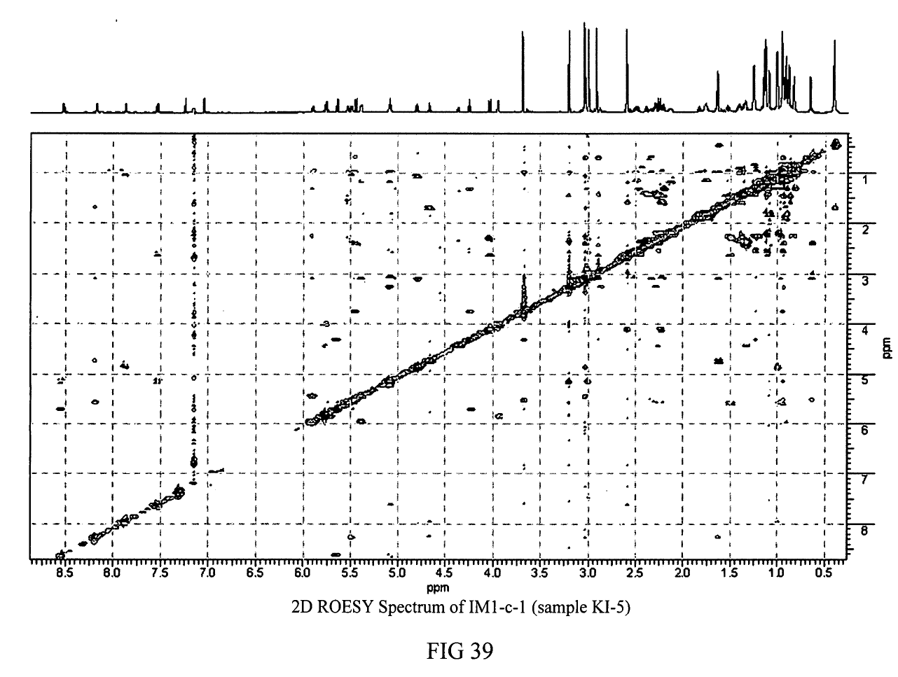

The stereochemistry at the E position of the side chain was deduced from the ROESY spectrum (see FIG. 39). The close examination of the ROESY spectrum for the aa-1 side chain protons showed the following ROE correlations:
δ 4.35 (1-ε)−δ 2.30 (1-δ₂) (1)
δ 4.25 (1-β)−δ 1.26 (1-γCH₃) (2)
δ 2.49 (1-γ)−δ 2.30 (1-δ₂) (3)
δ 1.35 (1-δ 1)−δ 1.26 (1-γCH₃). (4)

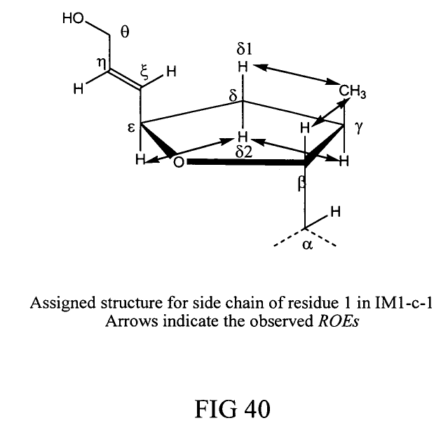

Based upon these observations, the aa-1 side chain structure for IM1-c-1 was elucidated (FIG. 40). Those of skill in the art will recognize that the ε carbon of aa-1 is a chiral center, and therefore, the ε carbon can be in the R configuration, as shown in FIG. 40, or in the S configuration, not shown. Based upon analysis of the ROESY spectrum, the metabolite isolated from the IM1-c-1 peak is predominantly in the R configuration. However, the metabolite shown in FIG. 40 may be existent in the S configuration as well.

Without wishing to be bound by any theory, based upon this structural analysis of the metabolites of ISA247, which resulted from metabolism at the amino acid-1 residue, the reaction scheme in FIG. 41 was formulated. This reaction scheme can begin with cis-ISA247 or trans-ISA247. A first step of the metabolism of ISA247 (1) is an epoxidation reaction. The epoxidation of ISA247 results in epoxides which can exist in conformations IM1-e-1, IM1-e-2 or IM1-e-3, shown in FIG. 41 as structures 2, 3 and 6, respectively. These epoxides were not identified by HPLC and have not been isolated. The epoxides appear to be extremely reactive. They may be intermediates or transition states in a reaction between ISA247 and the transformed products described above.

Without wishing to be bound by any theory, it is believed that once epoxides are formed, water can attack the epoxides to form diols. FIG. 42 illustrates proposed reaction mechanisms for the formation of ISA247 diol and cyclic aa-1 metabolites from trans ISA247. In FIG. 42, in reaction scheme 1 (see arrows labeled “1”) if water can attack the ε or ζ position of the IM1-e-1 epoxide, the epoxide opens and can form the IM1-d-4 metabolite. Note that the IM1-d-4 metabolite has two new chiral centers, at the ε and ζ carbons, so that the IM1-d-4 metabolite can exist in the form of the 4 diastereomers shown in FIG. 42. If water attacks the η or θ position of the trans epoxide, the epoxide IM1-e-2 opens to form the IM1-d-1 metabolite. Note that the η carbon of the IM1-d-1 metabolite is a chiral center. Therefore, the IM1-d-1 metabolite can exist as either diastereomer shown in FIG. 42. If the epoxide ring opening occurs concertedly with the double bond migration followed by water attack at the E position of the IM1-e-2 epoxide (see arrow labeled 2), the IM1-d-2 structure is formed, as shown in FIG. 42. Note that the ε carbon of IM1-d-2 is a chiral center. Therefore, the IM1-d-2 metabolite can exist as either of the diastereomeric isomers, as shown in FIG. 42. On the other hand, if the hydroxyl group of the β carbon of the IM1-e-2 epoxide attacks the ε carbon of the IM1-e-2 epoxide, the cyclic metabolite IM1-c-1 is formed. Again, note that the ε carbon is a chiral center, and the IM1-c-1 metabolite can exist as either of the diastereomers shown in FIG. 42.

Similarly, diol formation, proceeding from an intermediate epoxide, is possible starting from cis -ISA247. FIG. 43 shows a proposed reaction scheme for epoxide ring opening of a trans-epoxide IM1-e-2 (IM1-e-2trans) where IM1-e-2 indicates an amino acid 1 metabolite which is an epoxide, the second epoxide identified. FIG. 43 also shows a reaction scheme for epoxide ring opening of the cis-epoxide IM1-e-2 (IM1-e-2cis) to form 1,2-diols. The reaction from IM1-e-2trans results in the IM1-d-1 metabolite, as shown in FIGS. 42 and 43. In contrast, IM1-d-3 is formed from IM1-e-2cis, as shown in FIG. 43. Note that both IM1-d-1 and IM1-d-3 have a chiral center at the q carbon, and can therefore exist as either diastereomer.

Since the amino acid-1 side chain of ISA247 contains a conjugated diene system, with an extension of one carbon when compared to CsA, a greater number of diol and epoxide configurations are formed as metabolites in the ISA247 compound than for CsA. Since the terminal carbon of the amino acid-1 residue of the ISA247 molecule is part of an alkene functional group, there is not an ISA247 metabolite that is analogous to CsA-Am1, wherein the θ-carbon of the amino acid-1 side chain is monohydroxylated.

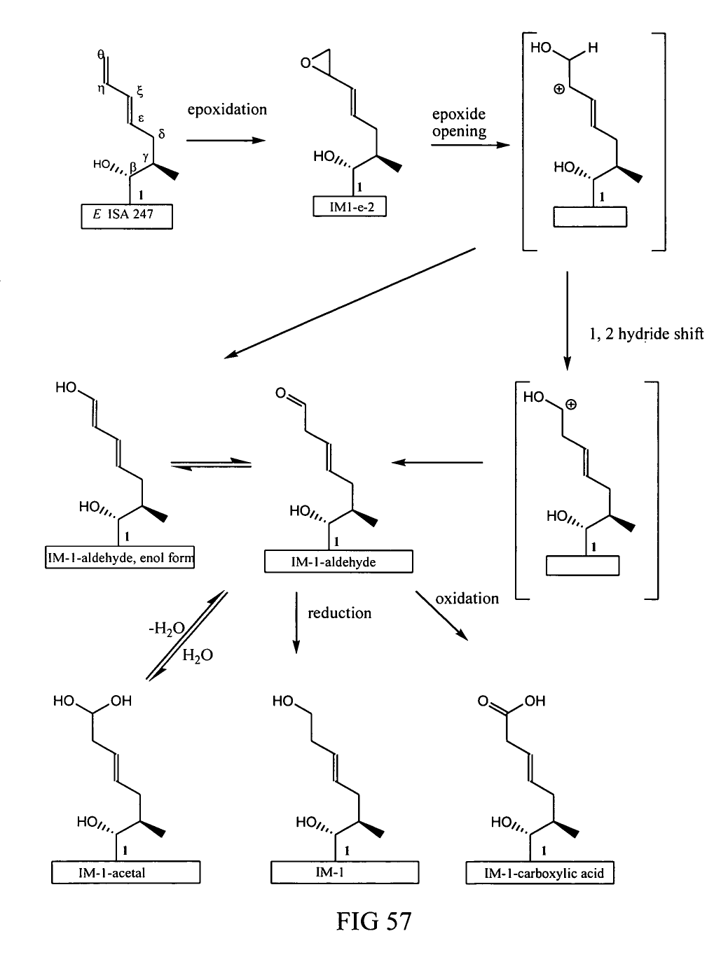

FIG. 57 is an exemplary reaction scheme illustrating the formation of IM-1, IM-1-acetal, IM1-aldehyde, and IM1-carboxylic acid from E-ISA247. Without wishing to be bound by theory, E-ISA247 is believed to be epoxidized at the η, θ alkene as described above to form IM1-e-2. The epoxide can open to form a cationic intermediate, which can undergo bond migration or a 1,2-hydride shift followed by formation of IM-1-aldehyde. The aldehyde can be reduced to form the alcohol IM-1, oxidized to form IM-1-carboxylic acid, or undergo H₂O addition to form IM1-acetal.

Table 5 shows a list of ISA247 metabolites which exhibit a modification at amino acid-1. Table 5 is not an exhaustive list. For example, amino acid 1 metabolites may include 5, 6, 7 or 8 member rings.

TABLE 5
Amino Acid-1 Metabolites of ISA247

| | |
|---|---|
| IM1-e-1 | 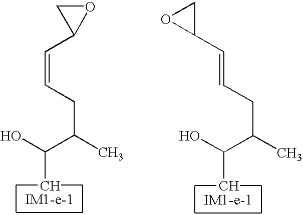 |
| IM1-e-2 | 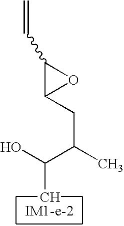 |
| IM1-e-3 | 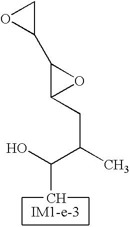 |
| IM1-d-1 | 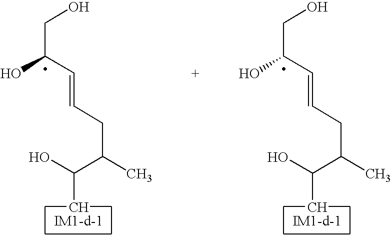 |
| IM1-d-2 | 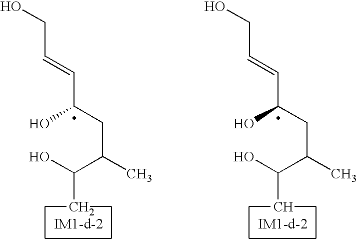 |
| IM1-d-3 | 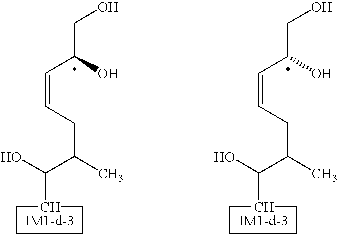 |
| IM1-d-4 | 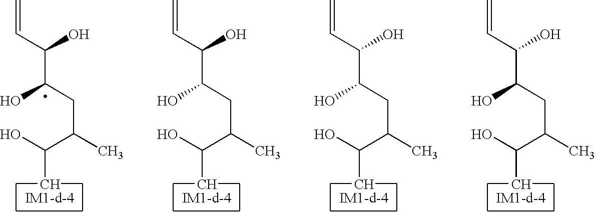 |
| IM1-c-1 | 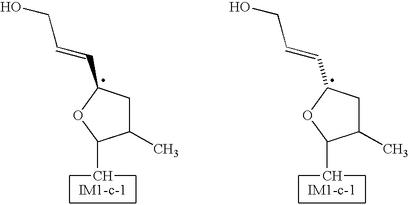 |
| IM-1 | 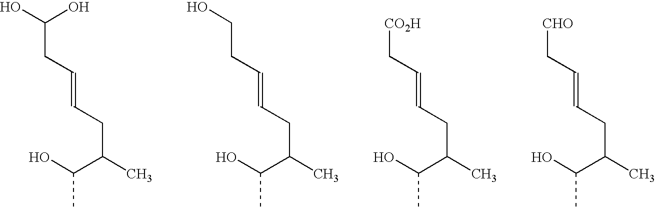 |
| IM1-c-2 | 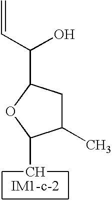 |

Hydroxylation Metabolites

The LCMS scan of FIG. 6 shows four peaks at the parent ion/fragment ion pair of 1253/1129 m/z. The major peak 1253/1129 ion pair having a chromatographic retention time of 8.5 minutes in FIG. 6 represents a hydroxylation on the side chain of an amino acid other than the amino acid-1. This is consistent with an IMX-type or hydroxylation-type metabolite at an amino acid other than the amino acid -1. Considering that the HPLC retention times of C_{S}A and ISA247 are identical and that the HPLC retention times of the 1253/1129 m/z ion pair peak and a C_{S}A-Am9 standard are also indistinguishable, it is likely that this metabolite is IM9.

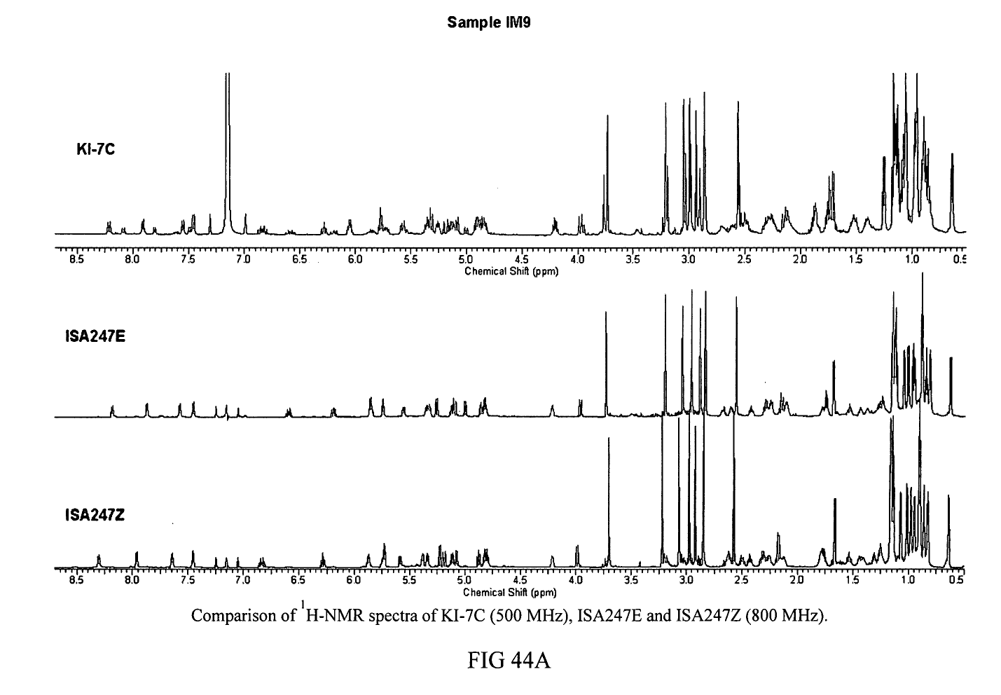

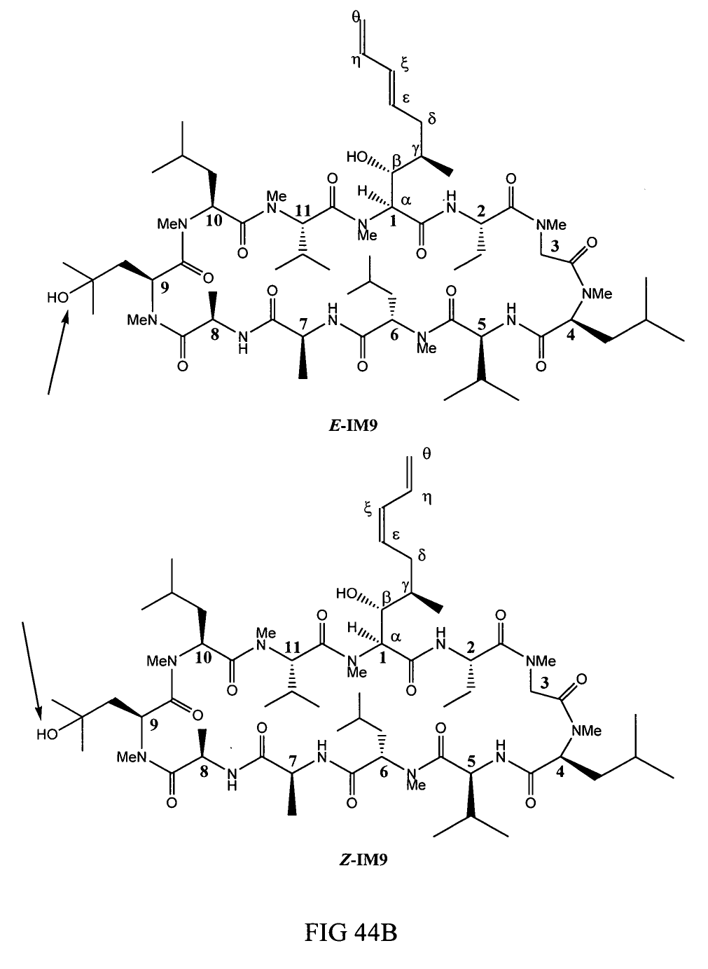

Sample KI-7C was obtained from microorganism transformation of ISA247 (cis:trans=1:1) and purified by HPLC. 1D ¹H-NMR spectra of KI-7C, E- and Z-isomers of ISA247 are shown in FIG. 44A. This comparison illustrates that the diene protons of the aa-1 between δ 6 and 7 ppm are not affected in this sample, indicating the aa-1 is not transformed. It also revealed that the sample is a mixture of Z and E isomers. Two sets of the 4 amide NH and the 7 N-methyl signals, arising from the E and Z isomers, were observed in the corresponding chemical shift regions, which indicated that the peptide ring is intact in this sample (KI-7C). Most of the amino acid protons were assigned by the 2D techniques (TOCSY and DQF-COSY, not shown). The only amino acid which lacked the connectivity to the side chain methyl group protons was aa-9. Therefore, combined with the mass analysis result of the hydroxylation, the aa-9 side chain transformation having the following structure was suggested:

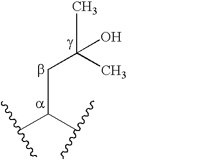

The above observation was further confirmed by ¹H-detected HMQC (Heteronuclear Multiple Quantum Correlation) and HMBC (Heteronuclear Multiple Bond Correlation) techniques (data not shown), which correlated both methyl protons with appropriate carbons through one and two to three bonds, respectively. The following shows HMBC correlation and the chemical shifts of methyl protons and relevant carbons of the amino acid-9 (aa-9) side chain, obtained from the above-mentioned heteronuclear correlation techniques. Thus, the NMR analysis of KI-7C demonstrated that sample KI-7C is IM9.

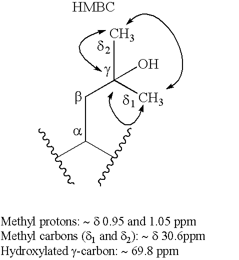

FIG. 44B shows the structure of IM9, a hydroxylation at the amino acid-9 residue. Note that the amino acid-1 side chain of the IM9 metabolite of ISA247 can exist in either the cis or trans configuration. Table 6 shows chemical shift assignments based on the 1H-NMR of sample KI-7C, the IM9 metabolite.

TABLE 6
¹H-NMR assignment of KI-7C (IM-9)^{1),2)}
KI-7C is a mixture of E and Z isomer (E:Z = 2:3)

| KI-7C (E) | | | KI-7C (Z) | | |
|---|---|---|---|---|---|
| | | Chemical | | | Chemical shift |
| Aminoacid | Hs | shift (ppm) | Amino acid | Hs | (ppm) |
| Amino acid-1 | | | Amino acid-1 | | |
| CH(α) | 1 | 5.79 | CH(α) | 1 | 5.77 |
| CH(β) | 1 | 4.21 | CH(β) | 1 | 4.21 |
| β-OH | 1 | 3.80 | β-OH | 1 | 3.47 |
| CH(γ) | 1 | 2.12 | CH(γ) | 1 | 2.12 |
| γ-CH3 | 3 | 1.12 | γ-CH3 | 3 | 1.12 |
| CH(δ1) | 1 | 2.72 | CH(δ1) | 1 | 2.70 |
| CH(δ2) | 1 | 2.31 | CH(δ2) | 1 | 2.50 |
| CH(ε) | 1 | 5.85 | CH(ε) | 1 | 5.73 |
| CH(ζ) | 1 | 6.20 | CH(ζ) | 1 | 6.29 |
| CH(η) | 1 | 6.59 | CH(η) | 1 | 6.84 |
| CH(θ1) | 1 | 5.09 | CH(θ1) | 1 | 5.19 |
| CH(θ2) | 1 | 5.01 | CH(θ2) | 1 | 5.08 |
| N—Me | 3 | 3.77 | N—Me | 3 | 3.74 |
| Amino acid-2 | | | Amino acid-2 | | |
| CH(α) | 1 | 5.14 | CH(α) | 1 | 5.13 |
| CH(β1) | 1 | ˜1.75 | CH(β1) | 1 | ˜1.76 |
| CH(β2) | 1 | ˜1.75 | CH(β2) | 1 | ˜1.76 |
| CH3(γ) | 3 | 0.85 | CH3(γ) | 3 | 0.86 |
| NH | 1 | 8.09 | NH | 1 | 8.22 |
| Amino acid-3 | | | Amino acid-3 | | |
| CH(α1) | 1 | 3.96 | CH(α1) | 1 | 3.98 |
| CH(α2) | 1 | 2.13 | CH(α2) | 1 | 2.15 |
| N—CH3 | 3 | 3.04 | N—CH3 | 3 | 3.05 |
| Amino acid-4 | | | Amino acid-4 | | |
| CH(α) | 1 | 5.55 | CH(α) | 1 | 5.58 |
| CH(β1) | 1 | 2.28 | CH(β1) | 1 | 2.33 |
| CH(β2) | 1 | 1.52 | CH(β2) | 1 | 1.52 |
| CH(γ) | 1 | 1.39 | CH(γ) | 1 | 1.39 |
| CH3(δ1) | 3 | 0.97 | CH3(δ1) | 3 | 0.98 |
| CH3(δ2) | 3 | 0.89 | CH3(δ2) | 3 | 0.90 |
| N—CH3 | 3 | 2.56 | N—CH3 | 3 | 2.57 |
| Amino acid-5 | | | Amino acid-5 | | |
| CH(α) | 1 | 4.84 | CH(α) | 1 | 4.84 |
| CH(β) | 1 | 2.61 | CH(β) | 1 | 2.61 |
| CH3(γ1) | 3 | 1.14 | CH3(γ1) | 3 | 1.14 |
| CH3(γ2) | 3 | 0.91 | CH3(γ2) | 3 | 0.91 |
| NH | 1 | 7.46 | NH | 1 | 7.46 |
| Amino acid-6 | | | Amino acid-6 | | |
| CH(α) | 1 | 5.33 | CH(α) | 1 | 5.36 |
| CH(β1) | 1 | 2.26 | CH(β1) | 1 | 2.26 |
| CH(β2) | 1 | 1.52 | CH(β2) | 1 | 1.52 |
| CH(γ) | 1 | 2.06 | CH(γ) | 1 | 2.08 |
| CH3(δ1) | 3 | 1.16 | CH3(δ1) | 3 | 1.17 |
| CH3(δ2) | 3 | 1.07 | CH3(δ2) | 3 | 1.08 |
| N—CH3 | 3 | 3.20 | N—CH3 | 3 | 3.21 |
| Amino acid-7 | | | Amino acid-7 | | |
| CH(α) | 1 | 4.89 | CH(α) | 1 | 4.88 |
| CH3(β) | 3 | 1.74 | CH3(β) | 3 | 1.71 |
| NH | 1 | 7.81 | NH | 1 | 7.92 |
| Amino acid-8 | | | Amino acid-8 | | |
| CH(α) | 1 | 4.91 | CH(α) | 1 | 4.91 |
| CH3(β) | 3 | 1.07 | CH3(β) | 3 | 1.06 |
| NH | 1 | 7.50 | NH | 1 | 7.56 |
| Amino acid-9 | | | Amino acid-9 | | |
| CH(α) | 1 | 6.05 | CH(α) | 1 | 6.05 |
| CH(β1) | 1 | 1.87 | CH(β1) | 1 | 1.87 |
| CH(β2) | 1 | 1.87 | CH(β2) | 1 | 1.87 |
| CH3(δ1) | 3 | ˜1.05³⁾ | CH3(δ1) | 3 | ˜1.05³⁾ |
| CH3(δ2) | 3 | ˜0.95³⁾ | CH3(δ2) | 3 | ˜0.95³⁾ |
| N—CH3 | 3 | 2.91 | N—CH3 | 3 | 2.94 |
| Amino acid-10 | | | Amino acid-10 | | |
| CH(α) | 1 | 5.26 | CH(α) | 1 | 5.26 |
| CH(β1) | 1 | 2.50 | CH(β1) | 1 | 2.50 |
| CH(β2) | 1 | 1.41 | CH(β2) | 1 | 1.41 |
| CH(γ) | 1 | 1.87 | CH(γ) | 1 | 1.87 |
| CH3(δ1) | 3 | 1.26 | CH3(δ1) | 3 | 1.26 |
| CH3(δ2) | 3 | 1.16 | CH3(δ2) | 3 | 1.16 |
| N—CH3 | 3 | 2.86 | N—CH3 | 3 | 2.87 |
| Amino acid-11 | | | Amino acid-11 | | |
| CH(α) | 1 | 5.34 | CH(α) | 1 | 5.32 |
| CH(β) | 1 | 2.26 | CH(β) | 1 | 2.26 |
| CH3(γ1) | 3 | 0.98 | CH3(γ1) | 3 | 0.97 |
| CH3(γ2) | 3 | 0.65 | CH3(γ2) | 3 | 0.65 |
| N—CH3 | 3 | 2.99 | N—CH3 | 3 | 3.00 |

¹⁾E and Z-isomer are assigned based upon the following coupling constants at aa-1.
E-isomer:
CH(ζ), δ 6.20(dd, 15.0 and 10.3Hz).
CH(η), δ 6.59(dt, 16.9, 10.3Hz).
Z-isomer:
CH(ζ), δ 6.29(t, 10.6Hz).
CH(η), δ 6.84(dt, 16.9 and 10.6Hz).
²⁾N-Methyl chemical shifts are assigned based upon the assignment on E-ISA247 and Z.
³⁾Obtained indirectly from the ¹H-detected HMQC and HMBC.

The material (sample KI-6) corresponding to the second HPLC peak with the parent ion/fragment ion pair of 1253/1129 m/z in the LCMS scan of FIG. 6, labeled IM4, was isolated from microorganism transformation of ISA247 (cis:trans=1:1) and analyzed using ¹H-NMR. Analogous mass information of this metabolite to IM9 indicated that KI-6 is also an IMX-type metabolite. The ¹H-NMR spectrum of KI-6 indicated that the metabolite is a mixture of two compounds, and the further examination of the amide NH proton (δ 7.5-8.7 ppm), the diene (δ 6.0-7.0 ppm) and the N-methyl region (δ 2.5-4.0 ppm), revealed that the metabolite is a mixture of E and Z-isomer at aa-1, having an E and Z ratio of 3:2. Interestingly, the starting material was in a 1:1 ratio of E and Z isomers of ISA247. This indicates that there may be differences in the rates of metabolism of E-ISA247 and Z isomers into this metabolite.

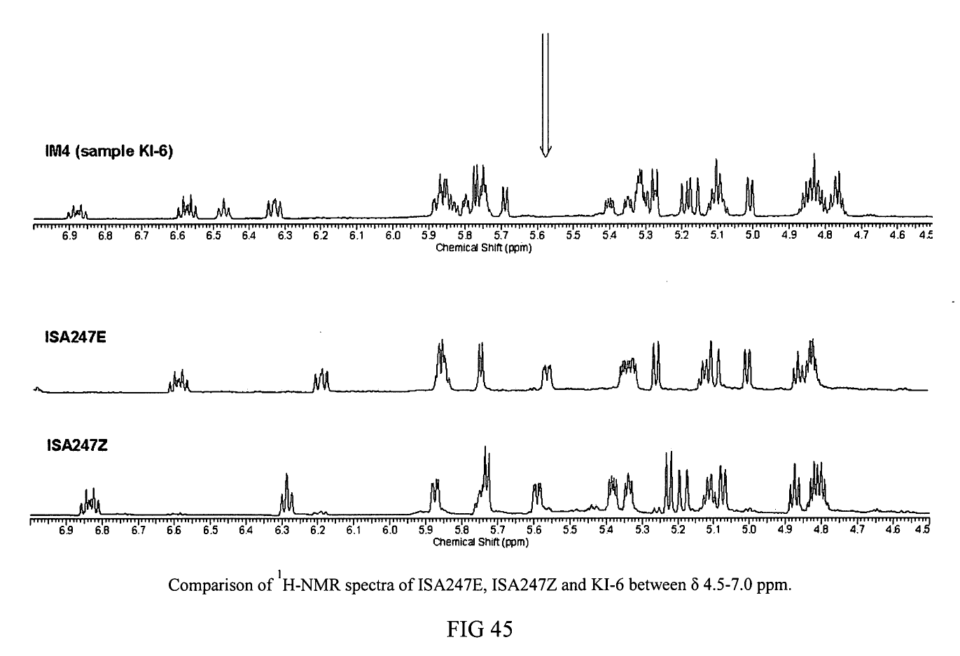

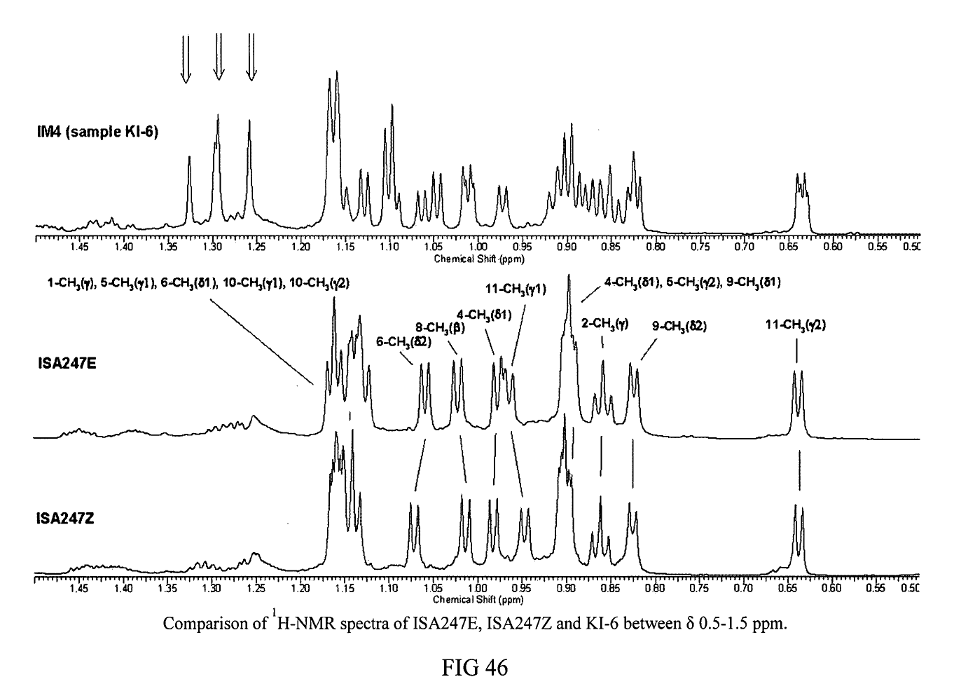

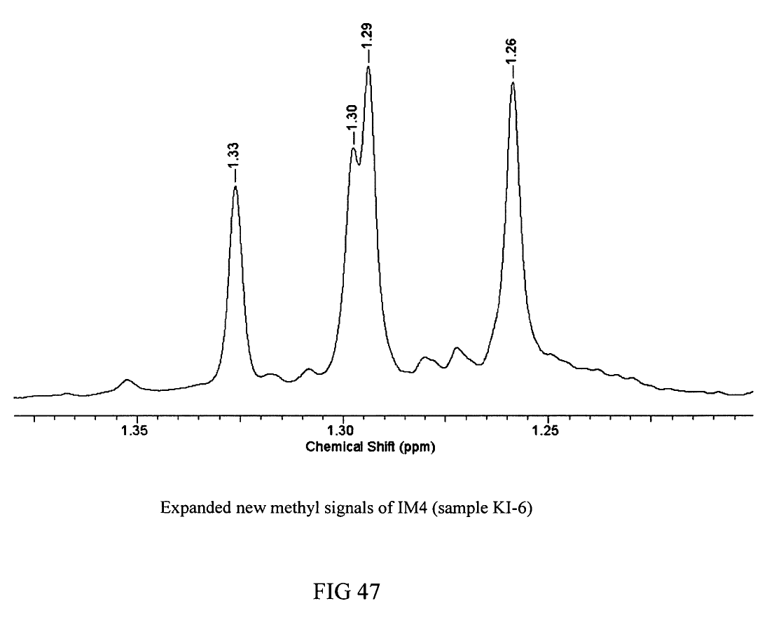

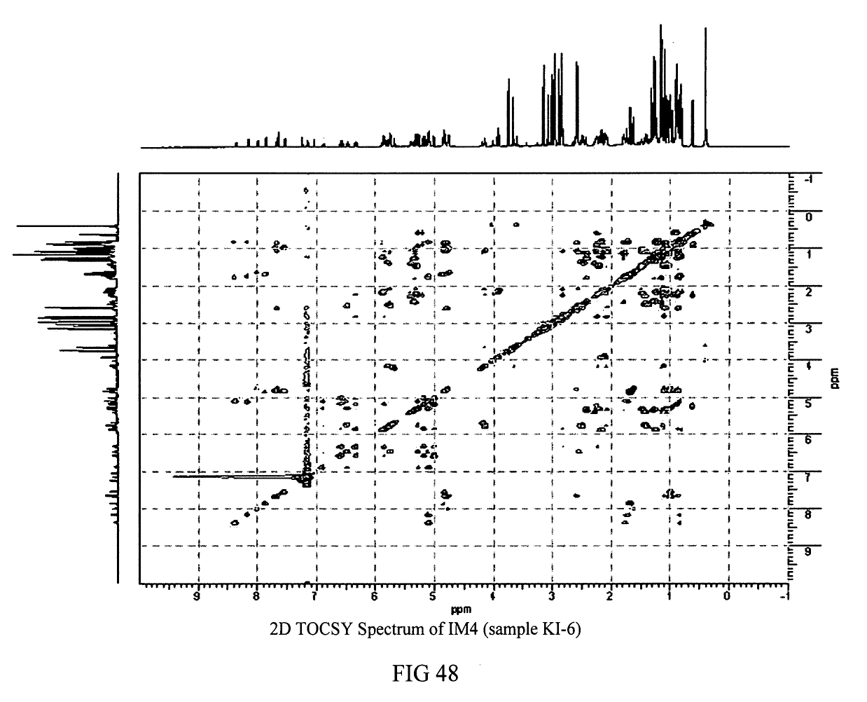

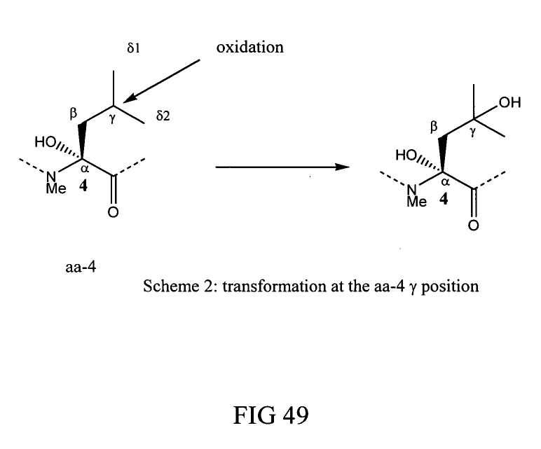

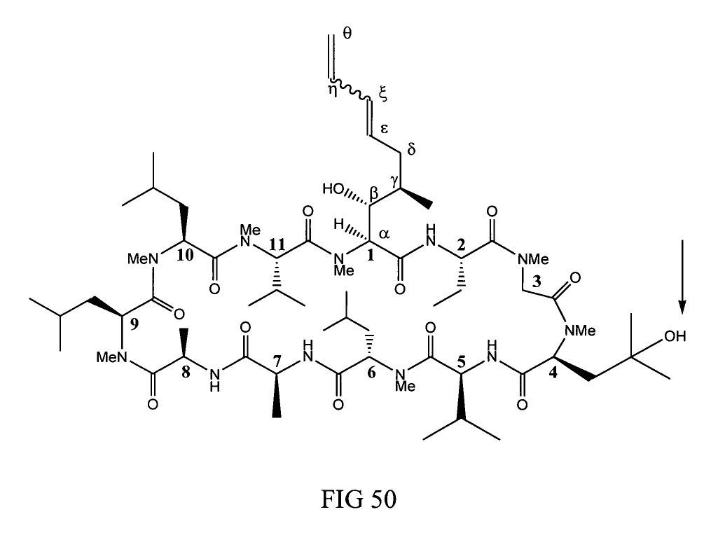

The existence of characteristic signals for the diene structures of ISA and seven pairs of N-methyl groups suggested that the side chain of aa-1 is intact and no N-demethylation had taken place. FIG. 45 is a comparison of the ¹H-NMR of the metabolite (labeled KI-6) and the ¹H-NMR spectra of E-ISA247 and Z-ISA247. Comparison of the NMR spectra in the diene and amino acid a proton region clearly showed the signals at δ 5.58 ppm, which correspond to the aa-4 α protons in the NMR spectra of Z-ISA247 and E, are absent or shifted to another location in the spectrum, as indicated by an arrow in FIG. 45. This observation suggested that the structural modification occurred near the α position of aa-4. Further NMR comparison of the side chain methyl proton region (δ 0.5-1.5 ppm) indicated the appearance of new methyl group signals, as pointed by arrows in FIG. 46. The expansion of these peaks, as shown in FIG. 46, revealed that there are two sets of singlet methyl signals at δ 1.33 and 1.30 ppm, and δ 1.29 and 1.26 ppm. The peak intensity ratio of 2:3 suggested that methyl signals at δ 1.33 and 1.30 ppm are of the Z-isomer, and those at δ 1.29 and 1.26 pm are of the E-isomer. FIG. 47 shows expanded new methyl signals of KI-6. The assignment of the methyl group peaks of ISA247 E and Z in the region illustrated in FIG. 46 showed that the methyl groups from the aa-4 side chain, 4-CH3 (δ 1) and. (δ 2), are absent from the KI-6 spectrum in the corresponding area, indicating that the new methyl peaks in FIG. 47 belong to the aa-4 side chain. Each methyl group of the aa-4 side chain normally appears as a doublet, since a methyl group is coupled to the γCH proton, as is the case for E-ISA247 and Z. Moreover, the mass study of the KI-6 sample indicated that a hydroxylation of an amino acid other than amino acid-1. Therefore, it was concluded that the observed singlet signals for δ methyl groups of aa-4 resulted from the oxidative transformation of ISA at the aa-4 γ position shown in FIG. 48. The 2D TOCSY spectrum (FIG. 49) also confirmed the above modification, providing the absence of signal connectivity for these methyl groups. This is consistent with KI-4 being IM4, γ-hydroxylation at amino acid-4. The structure of IM4 is shown in FIG. 50.

Although not isolated and analyzed for this study, hydroxylated metabolites, similar to IM9 and IM4 described above, may also occur at the γCH of amino acid-10 (EM10), at the γCH of amino acid-6(IM6), and also at the βCH of amino acid-5(IM5). Some of these hydroxylated metabolites were identified on the HPLC shown in FIG. 6, but not studies by NMR. For example, IMX(2) is a hydroxylated metabolite at an unknown amino acid.

N-Demethylation Metabolites

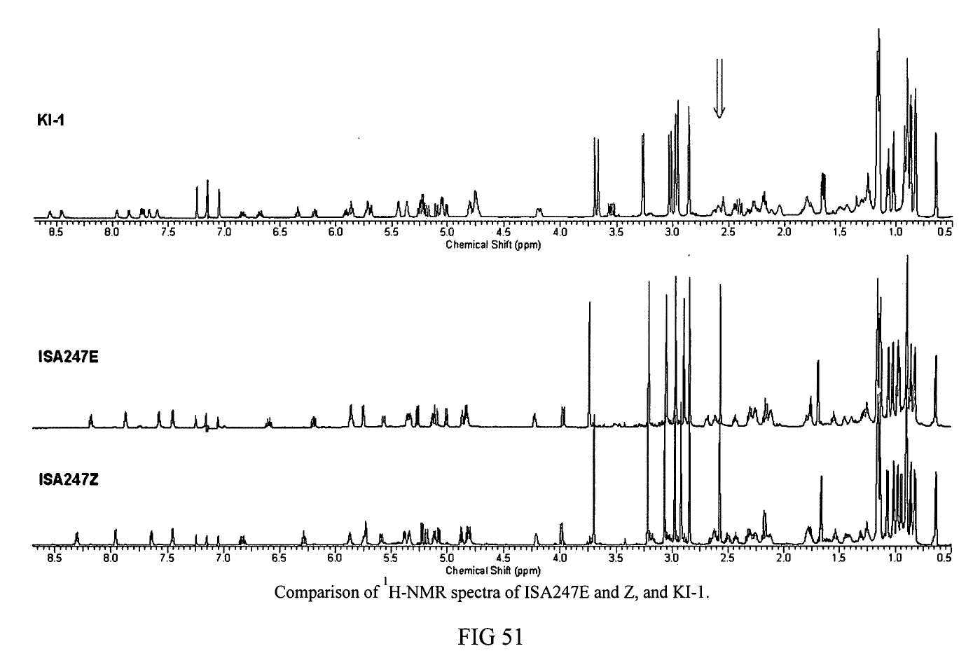

The isolated metabolite (sample KI-1) with the parent ion/fragment ion pair of 1223/1099 m/z, was analyzed by ¹H-NMR. The parent ion/fragmention pair of 1223/1099m/z of the metabolite suggested that it is an N-demethylated metabolite. The ¹H-NMR spectrum of metabolite KI-1 showed that the metabolite is a mixture of E and Z-isomer at about a 1:1 ratio, as evidenced by the presence of the diene proton peaks of the aa-1 side chain of both E- and Z-isomer, indicating that the aa-1 side chain is intact. The spectrum also revealed the loss of a pair of N-methyl peaks at δ 2.5 ppm, as indicated by an arrow in FIG. 51. Comparison of the ¹H-NMR spectrum of the metabolite with those of E-ISA247 and Z indicated that the missing signals corresponding to the region are those of the N-methyl groups (one from each isomer) of amino acid-4. Therefore, N-demethylation of amino acid-4 was suggested for the metabolite.

The signals of the aa-4 α protons of the metabolite were also shifted from those of the aa-4 α protons of ISA247 E and IDS247Z, which resonate at δ 5.56 and 5.59 ppm, respectively, and were not easily located in the spectrum. Furthermore, the amide NH proton region between δ 7 and δ 8.7 ppm only showed eight amide NH protons arising from both isomers, instead of ten, when two NH protons from aa-4 of both isomers were assumed in this region, therefore indicating that the amide NH protons of aa-4 resonate at a higher field.

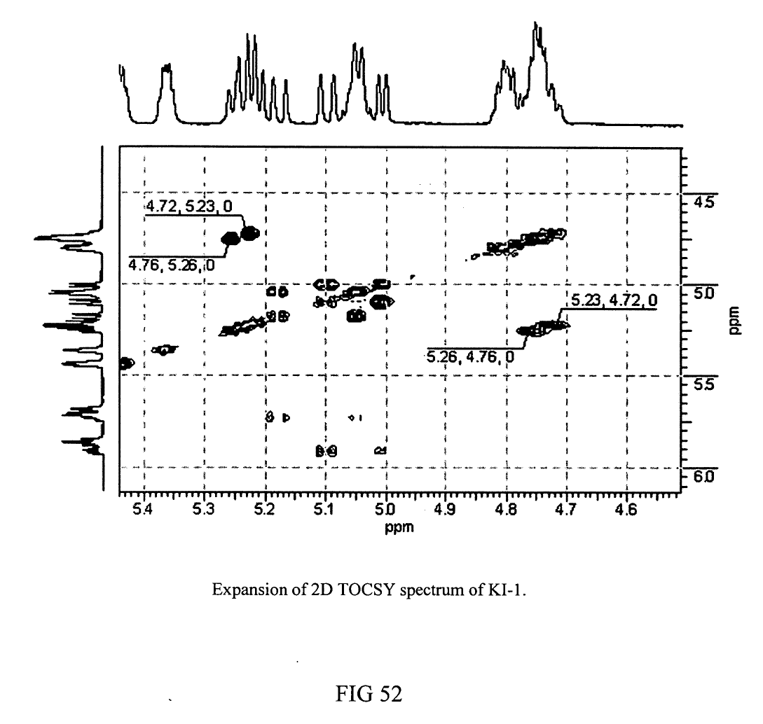

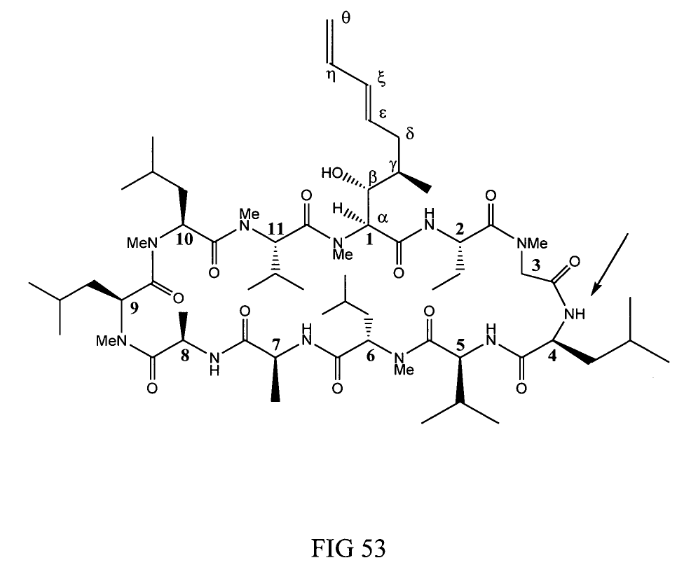

Analysis of the 2D TOCSY spectrum revealed two sets of signals at δ 5.2 ppm which correlate to those at δ 4.7 ppm. The expanded spectrum (FIG. 52) gives clear correlations of these signals: δ 5.26 ppm to 4.76 ppm, and δ 5.23 ppm to 4.72 ppm. Both sets of peaks are overlapped with other amino acid protons: those at δ 5.2 ppm with the aa-11 a protons and those at δ 4.7 ppm with the aa-5, aa-7 and aa-8 α protons. However, chemical shift assignments of aa-5, aa-7, aa-8 and aa-11 excluded the possibility of the signals at δ 5.2 and δ 4.7 ppm arising from these amino acids. It is suggested, therefore, that cross peak connectivity of δ 4.72 to 5.23 ppm and δ 4.76 to 5.26 ppm resulted from the amide NH and ax protons of aa-4 of both isomers. Based on this observation and confirmed by ¹H—¹⁵N HSQC (not shown), the signals at δ 5.26 ppm and 5.23 ppm were assigned for the NH protons, and those at δ 4.76 ppm and δ 4.72 ppm were assigned for the cc protons of aa-4. FIG. 53 shows a proposed structure of the metabolite KI-1 as IM4n.

Chemical synthetic methods can be used to produce metabolites of ISA247. Chemical synthesis of these metabolites generally follows the steps of: 1) protecting the β-OH of the 1 amino acid side chain of Cyclosporin A or ISA247; 2) epoxidation; 3) diol formation and 4) deprotection. While protection of the β—OH may be preferable for the formation of epoxide and diol metabolites, it is not consistent for the formation of cyclic metabolites. Possible protecting groups at the β-OH include acetyl, trimethylsilyl, benzoate esters, substituted benzoate esters, ethers and silyl ethers. Under certain reaction conditions, the acetate protecting group is prone to undesirable side reactions such as elimination and hydrolysis. Since benzoate esters, ethers and silyl ethers are often more resistant to such side reactions under those same reaction conditions, it is often advantageous to employ such protecting groups in place of acetate.

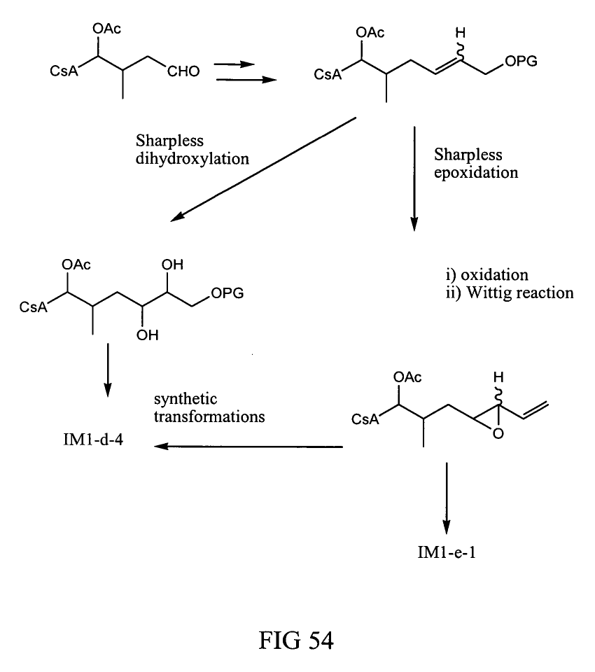

Under basic conditions, the protected CsA or ISA247 can form an epoxide. FIG. 54 illustrates a synthetic pathway using Sharpless methods (R. A. Johnson, K. B. Sharpless. Catalytic Asymmetric Synthesis: Edited by I. Ojima; VCH Publishers: New York; 1993; p. 103; K. B. Sharpless et al., J. Org. Chem. 1992, 57, 2768). As shown in FIG. 54, a protected CsA compound with an allylic alcohol moiety can undergo Sharpless epoxidation. A sequence of reactions involving oxidation, Wittig reaction and epoxide-ring opening, would lead to diol metabolites (See WO 2003/033526 and US 2003/0212249). Alternatively, a Sharpless dihydroxylation using commercially available reagent such as AD-mix-β/AD-mix-α (K. B. Sharpless etal J. Am. Chem. Soc. 1992, 114, 7570) could be employed in the synthesis of diol metabolites. Representative examples are illustrated in FIG. 54.

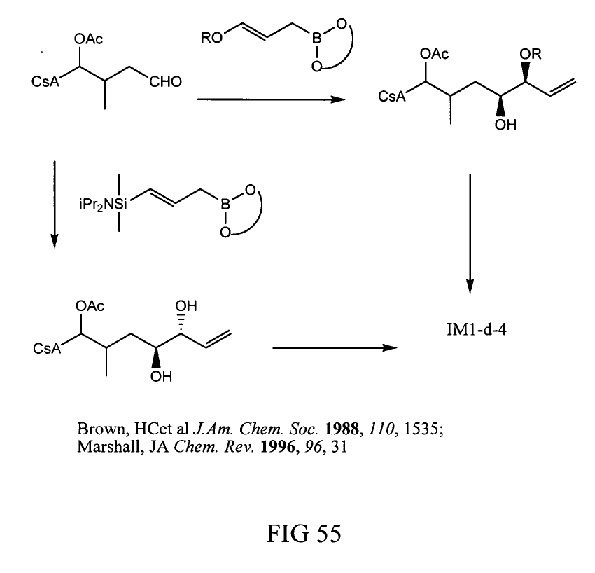

FIG. 55 illustrates that chemical synthetic methods can be used to direct the synthesis of specific syn or anti diols. For example, in FIG. 55, cis alkoxyallyl boronate ester reagents can be used to form the syn diol of IM1-d-4. The anti diol of IM1-d-4 can be formed if trans-silylallyl boronate ester reagents are used (see H. C. Brown, et al., J. Am. Chem. Soc. 1988, 110, 1535, Marshall, J. A. Chem. Rev. 1996, 96, 31, Barrett, A. G. M. et al., J. Org. Chem. 1991, 56, 5243).

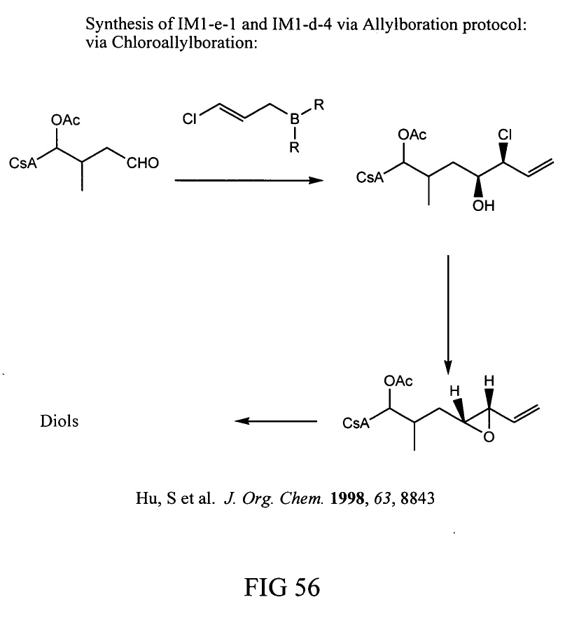

FIG. 56 illustrates the formation of epoxides (and diols) using chloroallylboration methods. FIG. 56 illustrates that the use of dialkyl (chloroallyl) borane reagents, using methods described in Hu et al., J. Org. Chem. 1998, 63, 8843 will result in the formation of cis-epoxides which can be transformed to diols, see also WO 2003/033526 and US 2003/0212249.

In addition to the cyclic amino acid-1 metabolites discussed above, which show 5 member ring formation, additional cyclic amino acid-1 metabolites are possible, which contain 6, 7, or 8 member rings. For example, in FIG. 42, cyclic metabolite formation is illustrated where the β-OH of the IM1-e-2 epoxide metabolite attacks its own ε-carbon in the presence of water. If the β-OH of the IM1-e-2 epoxide metabolite attacks its own ζ-carbon, a cyclic metabolite with a 6-member ring structure will be formed. If the β-OH of the IM1-e-2 epoxide metabolite attacks its own θ carbon, a cyclic metabolite with an 8 member ring structure is possible. Similarly, if the β-OH of the IM1-e-2 epoxide metabolite attacks its own η carbon, a cyclic metabolite with a 7 member ring structure is possible.

Demethylated metabolites, similar to IM4n described above, may also occur at the methylated nitrogens at aa-1, aa-3, aa-6, aa-9, aa-10 and aa-11. Some of these metabolites are identifiable on the HPLC scan of FIG. 6. For example, IMXn(2) is a demethylated metabolite, at an unknown amino acid in the ISA247 ring (designated “X”).

In addition to the metabolites described above, combinations of the metabolic steps may exist, creating metabolites with combinations of hydroxylations, N-demethylations, diol formations or cyclizations. For metabolites may exist which have multiple hydroxylations and N-demethylations. For example, IM-1-d-1 may be further metabolized with a demethylation of the nitrogen of MeLeu at position 4, creating IM1-d-1-4n. Other examples include IM1-d-2-4n or IM1-d-3-4n or IM1-d-4-4n, IM1-c-1-4n or IM1-c-2-4n. The demethylation which is combined with a diol formation or a cyclization may be on any amino acid on the ISA247 molecule which is amenable to N-demethylation. For example, metabolites of ISA247 include a diol formed at amino acid-1 (IM1-d-1, IM1-d-2, IM1-d-3 or IM1-d-4) combined with at least one N-demethylation at amino acid 1, 3, 4, 6, 9, 10 or 11. Metabolites of ISA247 may also include a diol formed at amino acid-1 combined with at least one hydroxylation at any amino acid on the ISA247 molecule that is amenable to hydroxylation. For example, metabolites of ISA247 include a diol formed at amino acid-1 (IM1-d-1, IM1-d-2, IM1-d-3 or IM1-d-4) combined with at least one hydroxylation at amino acid 4, 6, 9, 10, or 11. Metabolites of ISA247 may include a cyclization at amino acid (IM1-c-1 or IM1-c-2) combined with at least one N-demethylation at amino acid 1, 3, 4, 6, 9, 10 or 11, or at least one hydroxylation at amino acid 4, 6, 9, 10 or 11. Metabolites may have multiple hydroxylations, for example, IM46, IM69, or IM49, or multiple N-demethylations, for example IM4n9n or IM4n3n. Multiple metabolites are present in the HPLC scan of FIG. 6. For example, IMXnX(2) represents a metabolite which is both N-demethylated and hydroxylated at unidentified positions on the ISA247 ring. These multiple hydroxylations and/or multiple N-demethylations may also occur combined with a diol formation at aa-1, or a cyclization at aa-1.

In addition to the above-described Phase I metabolites, additional Phase II metabolites may occur. These Phase II metabolites may include groups such as glucuronide, saccharides (e.g., from glycosylation), phosphate, sulfate, and the like, which may occur at any hydroxyl group on the ISA247 or ISA247 metabolite molecule. Those of ordinary skill in the art will understand that this list of Phase II metabolites is not exhaustive, and that many additional Phase II metabolites are contemplated by this disclosure.

### EXAMPLE 1

### Preparation of ISA247 Metabolites from Whole Blood

Whole blood was taken from humans after administration of ISA247. ISA247 and its metabolites were extracted from whole blood using tertbutyl-methyl-ether (or methyl tertbutyl ether, MTBE), dried and reconstituted into methanol. 2 mL of MTBE (cat. No. 7001-2; Caledon) were added to 200 uL of blood, shaken for 10 minutes, and spun down in a table top centrifuge for 2 minutes. The top MTBE layer was removed and concentrated under vacuum. That residue was reconstituted in 200 uL of methanol. Bile and urine extractions can be performed similarly.

### EXAMPLE 2

### Chemical Synthesis of ISA247 Metabolites

Preparation of Monoepoxides of OAc-E-ISA247

To prepare diol metabolites of E-ISA247, epoxides were formed, as shown in FIG. 42. The following steps were carried out. To a stirred and cooled (0° C.) solution of OAc-E-ISA2⁴7 (125 mg, 0.1 mmol) in CHCl₃ (3 mL) was added potassium bicarbonate (10 mg). This was followed by addition of a solution of m-chloroperbenzoic acid (23 mg, 0.1 mmol, 77%) in CHCl₃ (2 mL). The reaction mixture was warmed to room temperature and stirring continued for 18 h. The reaction product was extracted with dichloromethane (25 mL). The organic layer was washed with saturated NaHCO₃ solution and brine. Drying (Na₂SO₄) and solvent removal furnished a white solid (110 mg). MS (m/z): 1295 (M+Na+). The product was a mixture of epoxides. The same process can be used with OAc-Z-ISA247 or a mixture of isomers of ISA247, but the stereochemistry of the products will be different, as shown in FIG. 42.

Cleavage of Epoxides of OAc-E-ISA247 to a Mixture of Diols:

The product above (110 mg) was added to a stirred and ice-cold mixture of acetone-water-88% HCO₂H (15 mL, 64.5:33:2.5) and stirred at room temperature for 72 h. The reaction mixture was worked up by extraction with ethyl acetate (25 mL), and the organic extract washed with saturated NaHCO₃ solution and brine. Drying (Na₂SO₄) and solvent removal furnished a white solid (110 mg). MS (m/z): 1313 (M+Na⁺). The product was a mixture of OAc-E-ISA247 diols.

Deprotection of Diols:

The mixture of OAc-E-ISA247 diols (110 mg) was dissolved in MeOH (10 mL) and water (4 mL) was added followed by solid potassium carbonate (110 mg). The reaction mixture was stirred for 36 h at room temperature and then extracted with ethyl acetate (25 mL). The combined organic extract was washed with brine and dried (NaSO₄). Removal of solvent gave a solid (110 mg) MS (m/z) 1271 (M+Na⁺). Purification using PHLC provided compounds IM1-d-1, IM1-d-2 and IM1-d-4.

### EXAMPLE 3

### Epoxidation of E-ISA247 (Preparation of Cyclic Metabolite):

In an acidic environment, cyclic compounds were formed. To a stirred and cooled (0° C.) solution of E-ISA247 (250 mg, 0.2 mmol) in CHCl₃ (3 mL) was added a solution of m-chloroperbenzoic acid (51 mg, 0.23 mmol, 77%) in CHCl₃ (2 mL) and stirred at room temperature for 48 h. The reaction mixture was cooled to 0C and excess m—CPBA was destroyed by addition of Me₂S (600 uL). The reaction product was extracted with dichloromethane (25 mL) and the organic layer was washed with saturated NaHCO₃ solution and brine. Drying (NaSO₄) and solvent removal furnished a solid (230 mg). The cyclized compounds, which were present in a mixture of IM1-c-1 and 1M1-c-2 were isolated using preparative HPLC.

### EXAMPLE 4

### Epoxidation of E-ISA247 (Preparation of Terminal Epoxide)

To a stirred and cooled (0° C.) solution of E-ISA247 (200 mg, 0.17 mmol) in CHCL₃ (3 mL) was added solid KHCO₃ (20 mg, 0.2 mmol). Then a soloution of m-chloroperbenzoic acid (45 mg, 0.2 mmol) in CHCl₃ (2 mL) was added. Stirring was continued at room temperature for a period of 4.5 h. The reaction mixture was then cooled in ice and Me₂S (500 uL) was added. Work up and HPLC separation as above furnished the terminal epoxide, IM 1-e-1.

### EXAMPLE 5

### ISA247 Metabolite Production by a Dog Liver Microsome Preparation

Preparation of Dog Microsomes

Dog liver microsomes were prepared in the following manner: after removing the liver, it was flushed with 1.15% potassium chloride (KCl); diced into small pieces (approximately 25 g ) and ground until major chunks were disintegrated in a chilled grinding buffer (0.1 M phosphate buffer pH 7.4; 4° C.; 1:1 ratio of buffer to liver). A Polytron Homogenizer (15,000 rpm for 3 to 5 minutes) was utilized to form a homogenate, which contained liver tissue. After decanting the supernatant from the particulate matter, the supernatant was centrifuged for 90 min. at 100,000×g to yield a microsomal pellet. Protein content of the microsomal pellet was determined using the Lowry protein assay. The protein concentration of this microsomal preparation was approximately 23.2 mg/mL. To avoid enzyme activity loss, microsomes were stored in 4.0 or 6.0 mL aliquots at −80° C. to avoid freeze thaw cycling.

A 6 mL volume of dog liver microsome, prepared as above, was incubated in a 257 mL Erlenmeyer Flask with the following ingredients added stepwise: 57.3 mg of NADP, 254 mg of Glucose-6-Phosphate, and 23.0 mg NADPH were added to 6.0 mL of Phosphate Buffer (adjusted to pH 7.4). Then, 2.0 mL of 5.0 mM MgCl, and 6.0 mL Glucose-6-Phosphate Dehydrogenase (10 units/mL, available from CALBIOCHEM, San Diege, Calif., Cat. No. 346774) were added to the solution. Finally, 10 mL of Phosphate Buffer (pH 7.4) was added. The flask was incubated at 37° C. for 2 hours at 250 rpm in an environmentally controlled incubator/shaker. At 2 hours, the reaction was stopped by adding 500 μL of 2M HCl.

Metabolites produced by this method were then extracted with an organic solvent, and further separated using high-pressure liquid chromatography (HPLC). Metabolites were further characterized by electrospray mass spectrometry (MS) and NMR.

### EXAMPLE 6A

### ISA247 Metabolite Production by Biotransformation

The biotransformation system utilized microorganisms containing the microbial equivalent of the human cytochrome P450 microsomal enzymes and a medium suitable for active growth of the microorganism. The parent compound, which is poorly soluble in water, was mixed with ethanol and a surfactant prior to addition to the biotransformation system. In this example, ISA247 in ethanol was mixed with Tween 40 and then added to a biotransformation system containing Saccharopolyspora erytheraea ATCC 11635.

A biotransformation experiment was initiated with 15 slants of Saccharopolyspora erytheraea. These slants were prepared from 100 mL of ATCC medium 196 (approximately 6.0 mL per slant) which was dissolved in deionized water, adjusted to pH 7.0 with NaOH, and sterilized for 30 minutes at 100° C. Following inoculation with Saccharopolyspora erytheraea the slants were allowed to grow for three weeks at 28° C.

Colonies from these slants were then transferred to Phase I Media. Phase I Media was prepared with 10 g/L dextrin, 1 g/L glucose, 3 g/L beef extract, 10 g/L yeast extract, 5 g/L magnesium sulfate and 400 mg/L potassium phosphate. These ingredients were mixed in deionized water up to 1 liter, and adjusted to pH 7.0 with NaOH. 50 mL aliquots were then transferred to baffled 250 mL culture flasks and sterilized for 30 min. at 100° C. To initiate a Phase I culture, 5 mL of media was aliquoted into the agar slant containing Saccharopolyspora erythraea. Cells were scraped off the surface of the slant forming a cellular suspension. 2.5 mL of this suspension was used to inoculate each flask. The flasks were placed on a Labline Incubator at 27° C. and shaken at 250 rpm for 3 days (72 hrs).

Saccharopolyspora erythraea was transferred to Phase II media from Phase I medium by centrifuging the contents of a Phase I flask at 3300 rpm for 5 min. and decanting off the supernatant to obtain a pellet. 5 mL of Phase II media was added to the pellet and the tube was vortexed, then centrifuged at 3300 rpm for 4 min. Again the supernatant was decanted. The pellet was resuspended in Phase II media. The subsequent suspension was added to 50 mL of Phase II medium in a baffled culture flask.

Phase II Media contained with 10 g/L glucose, 1 g/L yeast extract, 1 g/L beef extract and 11.6 g/L of 3-N-morpholinopropanesulfonic acid (MOPS) buffer. These ingredients were mixed in deionized water and adjusted to pH 7.0 with 5M NaOH. 50 mL aliquots were dispensed into baffled culture flasks (250 mL) and autoclaved for 30 min. at 100° C. Tween 40 was also autoclaved.

ISA247 (4 mg) was dissolved in 0.1 ml Ethanol (95%) then mixed with 0.4 ml Tween® 40 (polyoxyethylene sorbitan monopalmitate; Cat. No. P1504. Sigma-Aldrich, St. Louis, Mo.) The parent compound-surfactant mixture was then added to Saccharopolyspora erythraea in the Phase II culture medium. A zero time sample was obtained and frozen. Each flask was then capped and placed on an Innova Incubator at 27° C. and incubated for 120 hrs with shaking at 170 rpm.

A second sample was obtained from the Phase II culture medium. The zero time sample and the second sample were extracted using tert-butyl-methyl ether (cat. No. 7001-2; Caledon). The extracted metabolites were reconstituted in methanol (HPLC grade) and analyzed by LC-MS as described below.

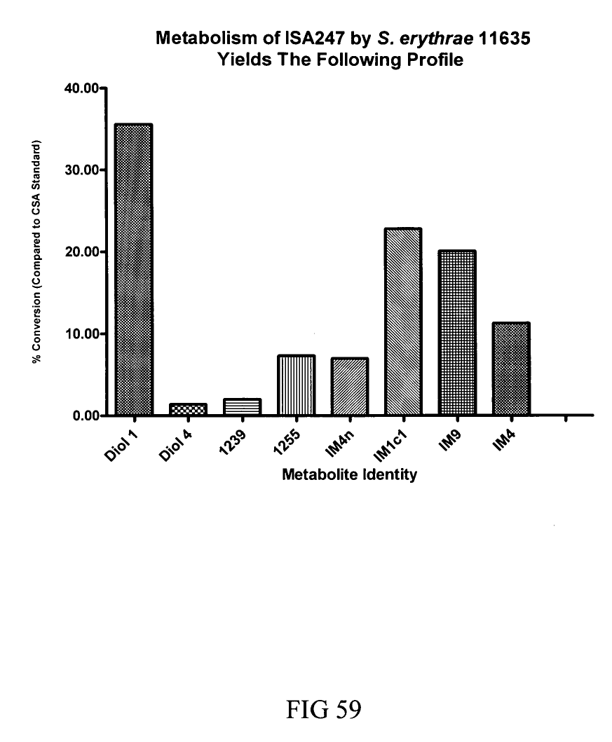

FIG. 59 is a bar graph exemplifying the amounts and types of metabolites which can be produced by (i.e., the metabolic diversity of) ATCC 11635.

### EXAMPLE 6B

### ISA247 Metabolite Production by Biotransformation

In further experiments based on the preceding biotransformation example, a variety of microorganisms were evaluated for production of ISA247 metabolites from ISA247, including Curvularia lunata (UAMH 9191, ATCC 12017), Cunninghamella echinulata var. elegans (UAMH 7370, ATCC 36112), Curvularia echinulata var. blakesleena (UAMH 8718, ATCC 8688a), Cunninghamella echinulata var. elegans (UAMH 7369, ATCC 26269), Beauvaria bassiana (UAMH 8717, ATCC 7159), Actinomycetes ((ATCC 53828), Actinoplanes (ATCC 53771), Cunninghamella echinulata (UAMH 4144, ATCC 36190), Cunninghamella echinulata (UAMH 7368, ATCC 9246), Cunninghamella bainiere (echinulata) (UAMH 4145, ATCC 9244) and Saccharopolyspora erythrae (ATCC 11635).

These microbes were screened for metabolite conversion yield (total amount of known ISA247 metabolites produced versus starting ISA24) as well as metabolic diversity (number of different ISA247 metabolites produced). Additionally, delivery adjuvants (to increase uptake of the highly lipophilic ISA247) were examined, including dimethyl sulfoxide (DMSO) and Tween 40, in comparison to glycerol, a known adjuvant. Samples were taken from the media and analyzed with LC-MS against a human standard ISA247 metabolite profile as described below. Table 7 lists the ion masses found, corresponding quantifiable ISA247 metabolites and approximate retention times. Ion masses quantified included 1223, 1237, 1239, 1253, 1255, 1267 and 1271.

TABLE 7

| | | Approximate Retention |
|---|---|---|
| Ion Mass | Metabolite from ISA247 | Time (min) |
| 1223 | IM4n | 9.789 |
| 1237 | ISA247 | 10.206 |
| 1239 | 1239 | 8.340 |
| 1253 | IM1-c-1; IM9; IM4 | 8.575; 8.939; 9.440 |
| 1255 | 1255 | 8.899 |
| 1267 | CSA(Internal Standard) | 10.678 |
| 1271 | IM1-d-1; IM1-d-4 | 7.535; 8.166 |

Table 8 ranks the microbes tested based on total conversion and metabolic diversity after 96 hours of biotransformation. A check mark indicates a quantifiable amount of metabolite was produced.

TABLE 8

| | ATCC | UAMH | ATCC | ATCC | UAMH | UAMH | UAMH | UAMH | UAMH |
|---|---|---|---|---|---|---|---|---|---|
| Metabolite | 11635 | 4145 | 53771 | 53828 | 7369 | 7370 | 8717 | 8718 | 9191 |
| IM1-d-1 | ✓ | ✓ | ✓ | | ✓ | ✓ | | ✓ | ✓ |
| IM1-d-4 | ✓ | | | | | | ✓ | | ✓ |
| 1239 | ✓ | | ✓ | ✓ | | ✓ | ✓ | ✓ | ✓ |
| 1255 | ✓ | ✓ | ✓ | | | | ✓ | | |
| IM4n | ✓ | ✓ | ✓ | | ✓ | | | ✓ | ✓ |
| IM1-c-1 | ✓ | ✓ | ✓ | ✓ | ✓ | ✓ | | ✓ | ✓ |
| IM9 | ✓ | ✓ | | ✓ | ✓ | ✓ | | ✓ | ✓ |
| IM4 | ✓ | ✓ | | ✓ | | | | ✓ | |
| Rank | 1st | 4th | 3rd | 9th | 8th | 7th | 6th | 5th | 2nd |

### EXAMPLE 7

### LC/MS Methodology for the Analysis of ISA247 Metabolites

In this example, ISA247 metabolites were produced in vitro, separated using high-pressure liquid chromatography (HPLC), and characterized using electrospray mass spectrometry.

Liquid Chromatographic (LC) Conditions

For Liquid Chromatography (LC or HPLC) a reverse phase Waters Symmetry C8, 2.1×50 mm, 3.5 cm analytical column (Waters cat# WAT 200624) with a guard column 2×20 mm (Upchurch Scientific cat# C-130B) packed with Perisorb RP-8 (Upchurch Scientific cat# C-601) was used. The solvent percentages and flow rates utilized in the LC program are given in Table 9:

TABLE 9

| | 0.2% GAA + | | |
|---|---|---|---|
| Time | 10⁻⁵M Na | MeOH:MtBE (9:1) | Flow rate |
| (min) | Acetate (%) | (%) | (mL/min) |
| 0.00 | 55 | 45 | 0.5 |
| 5.00 | 45 | 55 | 0.5 |
| 10.00 | 5 | 95 | 0.5 |
| 12.00 | 5 | 95 | 0.5 |
| 12.01 | 55 | 45 | 0.5 |
| 15.00 | 55 | 45 | 0.5 |

Mass Spectral (MS) Conditions

For Mass Spectrometry, an Applied Biosystems/MDS Sciex API3000 (Analyst software v 1.2) machine was used. Run time was 15 minutes, injection volume was 5 μL, Guard Column Temperature and Analytical Column Temperatures were 60° C. Manual settings were as follows: Turbo Ion Spray was 8000, Turbo Ion Spray horizontal setting was positive 4, Turbo Ion Spray lateral setting was 10. The Sciex machine was set with the parameters shown in Table 10.

TABLE 10
MS Settings
MS Settings:

| Scan type: | MRM (Multiple Reaction Monitoring) |
|---|---|
| Polarity: | Positive |
| Period Duration | 15.00 min |
| Period Cycle: | 1.32 sec |
| # of Cycles: | 692 |

Advanced MS Settings:

| | | |
|---|---|---|
| Resolution | Q1: | Low |
| | Q3: | Low |

| | |
|---|---|
| Intensity threshold: | 0 |
| Settling time: | 50 msec |
| Pause time: | 30 msec |

Parameter Settings:

| | |
|---|---|
| Ion Source: | Turbo ion spray |
| Nebulizer Gas: | 12 |
| Curtain Gas: | 8 |
| Collision Gas: | 12 |
| Ion Spray voltage: | 5000 V |
| Temperature: | 550° C. |

Compound Settings:

| | |
|---|---|
| Declustering Potential: | 60 V |
| Focusing Potential: | 400 V |
| Collision Energy: | 90 V |

Table 11 shows ions and ion-specific instrument settings.

TABLE 11

| Q1 Mass | Q3 Mass | Time |
|---|---|---|
| (amu) | (amu) | (msec) |
| 1222.8 | 1098.7 | 100 |
| 1236.8 | 1112.7 | 100 |
| 1252.8 | 1128.7 | 100 |
| 1252.8 | 1224.7 | 100 |
| 1270.8 | 1112.7 | 100 |
| 1254..8 | 1130.7 | 100 |
| 1268.8 | 1128.7 | 100 |
| 1268.8 | 1144.7 | 100 |
| 1238.8 | 1114.7 | 100 |
| 1268.8 | 1240.8 | 100 |

### EXAMPLE 8

### Measurement of Immunosuppressive Activity of ISA247 Metabolites

Calcineurin activity was assayed using a modification of the method previously described by Fruman et al. (A Proc Natl Acad Sci USA, 1992) and in U.S. Pat. No. 6,605,593. Whole blood lysates are evaluated for their ability to dephosphorylate a ³²P-labelled 19 amino acid peptide substrate in the presence of okadaic acid, a phosphatase-type 1 and 2 inhibitor. Background phosphatase 2C activity (CsA and okadaic acid resistant activity) is determined and subtracted from each sample, with the assay performed in the presence and absence of excess added ISA247. The remaining phosphatase activity is taken as calcineurin activity.

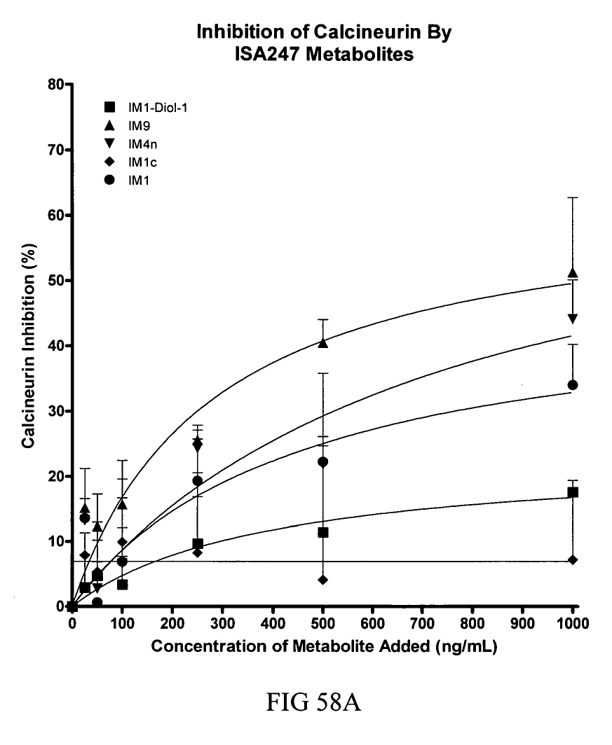

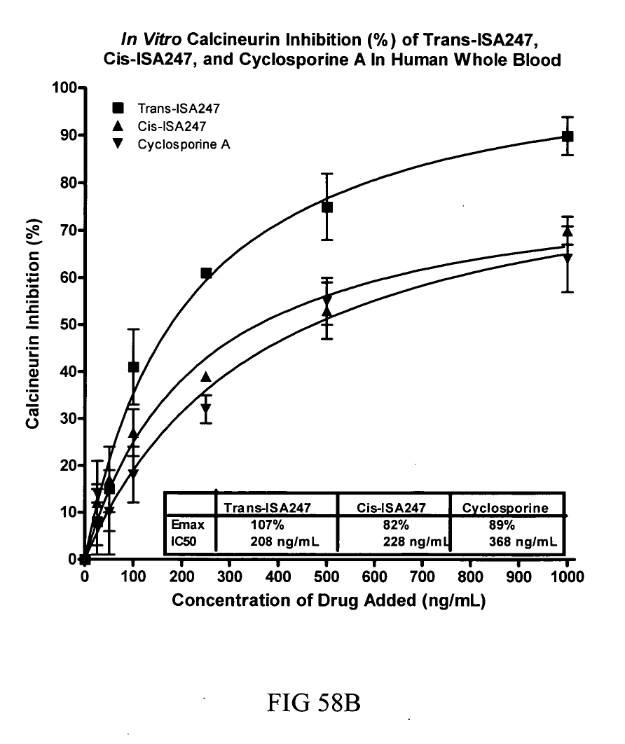

FIG. 58A is a graph of showing percent calcineurin inhibition versus concentration of metabolite added in ng/mL. Calcineurin inhibition as a function of concentration by ISA247 metabolites IM1-diol-1, IM9, IM4n, IM1c, and IM1 is comparable to trans-ISA247, cis ISA247, and CsA, as can be seen by contrasting FIG. 58A with FIG. 58B, which shows calcineurin inhibition versus concentration for trans-ISA247, cis ISA247, and CsA. Table 12 shows Emax and EC50 of IM1, IM1-diol-1, IM4n, and IM9 compared to trans ISA247 and CsA.

TABLE 12

| Metabolite I.D. | Metabolite Emax | Metabolite EC50 |
|---|---|---|
| IM1 | 47.6% | 450.0 ng/mL |
| IM1-Diol-1 | 23.3% | 394.5 ng/mL |
| IM4n | 71.5% | 720.9 ng/mL |
| IM9 | 62.9% | 271.3 ng/mL |
| Trans ISA247 | 107% | 208 ng/mL |
| Cyclosporine A | 89% | 368 ng/mL |

All of the publications, patents and patent applications cited in this application are herein incorporated by reference in their entirety to the same extent as if the disclosure of each individual publication, patent application or patent was specifically and individually indicated to be incorporated by reference in its entirety.

Reaction mechanisms herein, whether chemical or enzymatic, are theoretical and are provided to clarify and exemplify the processes described herein. While such mechanisms are believed to be true, one of skill in the art can appreciate that future evidence can result in modification of such mechanisms. Thus, Applicants do not intend that the embodiments disclosed herein be bound by such theoretical mechanisms.

Many modifications of the exemplary embodiments of the invention disclosed above will readily occur to those skilled in the art. Accordingly, the invention is to be construed as including all structure and methods that fall within the scope of the appended claims.

What is claimed is:

1. An isolated compound represented by the following formula: 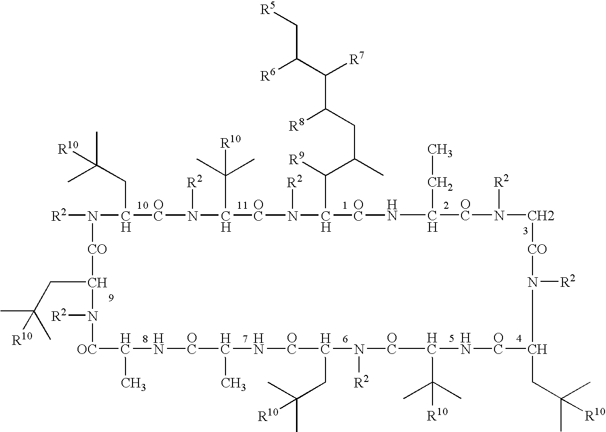
and pharmaceutically acceptable salts and solvates thereof, wherein:
each R² is independently —H or —CH₃;
each R¹⁰ is independently —H, —OH, —F, —Cl, —Br, —I, —CN, —NO₂, —OR^{a}, —C(O)R^{a}, —OC(O)R^{a}, —C(O)OR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a} or —NR^{c}C(O)N(R^{a}R^{b});
R⁵, R⁶, R⁷, R⁸ and R⁹ are independently —H, —OH, —F, —Cl, —Br, —I, —CN, —NO₂, —OR^{a}, —C(O)R^{a}, —OC(O)R^{a}, —C(O)OR^{a}, —S(O)R^{a}, —SO₂R^{a}, —SO₃R^{a}, —OSO₂R^{a}, —OSO₃R^{a}, —PO₂R^{a}R^{b}, —OPO₂R^{a}R^{b}, —PO₃R^{a}R^{b}, —OPO₃R^{a}R^{b}, —N(R^{a}R^{b}), —C(O)N(R^{a}R^{b}), —C(O)NR^{a}NR^{b}SO₂R^{c}, —C(O)NR^{a}SO₂R^{c}, —C(O)NR^{a}CN, —SO₂N(R^{a}R^{b}), —SO₂N(R^{a}R^{b}), —NR^{c}C(O)R^{a}, —NR^{c}C(O)OR^{a} or —NR^{c}C(O)N(R^{a}R^{b}); or R⁶ and R⁷ are together —O—; or R⁵ and R⁶ together, or R⁷ and R³ together, are independently —O—; or R⁸ and R⁹ together are —O—; or R⁵, together with the carbon to which it is bonded, is —C(═O)R^{a}, —CO₂R^{a}, —CH₂OR^{a}, —CH₂OC(O)R^{a}, —CH(OR^{a})₂, —C(O)N(R^{a}R^{b}), —C(═NR^{b})R^{a}, —C(═NOR^{b})R^{a}, or —C(═NNR^{b})R^{a}; provided that one pair of R⁵ and R⁶, R⁶ and R⁷, or R⁷ and R⁸ is a carbon-carbon bond and the remainder are not all —H; and
R^{a}, R^{b} and R^{c} are each independently —H or an optionally substituted aliphatic, cycloaliphatic, benzyl, or aryl, or —N(R^{a}R^{b}) together is an optionally substituted heterocyclic group, or —CH(OR^{a})₂ together is a cyclic acetal group.

2. The isolated compound of claim 1, wherein the compound is represented by the following formula: 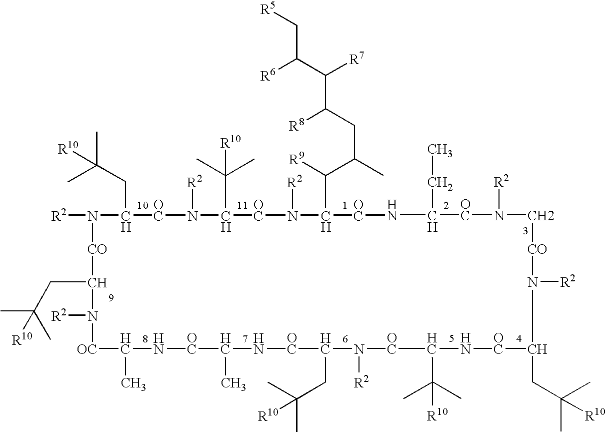
wherein:
each R² is independently —H or —CH₃;
each R¹⁰ is independently —H, —OH, —F, —Cl, —Br, —I, —OR^{a}, —OC(O)R^{a}, —OSO₂R^{a}, —OSO₃R^{a}, —OPO₂R^{a}R^{b} or —OPO₃R^{a}R^{b};
R⁵, R⁶, R⁷, R⁸ and R⁹ are independently —H, —OH, —F, —Cl, —Br, —I, —OR^{a}, —OC(O)R^{a}, —OSO₂R^{a}, —OSO₃R^{a}, —OPO₂R^{a}R^{b} or —OPO₃R^{a}R^{b}; or R⁶ and R⁷ are together —O—; or R⁵ and R⁶ together, or R⁷ and R⁸ together, are independently —O—; or R⁸ and R⁹ together are —O—; or R⁵, together with the carbon to which it is bonded, is —C(═O)R^{a}, —CO₂R^{a}, —CH₂OR^{a}, —CH₂OC(O)R^{a}, —CH(OR^{a})₂, —C(O)N(R^{a}R^{b}), —C(═NR^{b})R^{a}, —C(═NOR^{b})R^{a} or —C(═NNR^{b})R^{a}; provided that one pair of R⁵ and R⁶, R⁶ and R⁷, or R⁷ and R⁸ is a carbon-carbon bond and the remainder are not all —H; and
R^{a}, R^{b} and R^{c} are each independently —H or an optionally substituted aliphatic, cycloaliphatic, benzyl, or aryl, or —N(R^{a}R^{b}) together is an optionally substituted heterocyclic group, or —CH(OR^{a})₂ together is a cyclic acetal group.

3. The isolated compound of claim 1, wherein the compound is represented by the following formula: 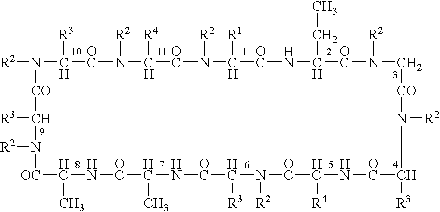
wherein:
R¹ is selected from the group consisting of 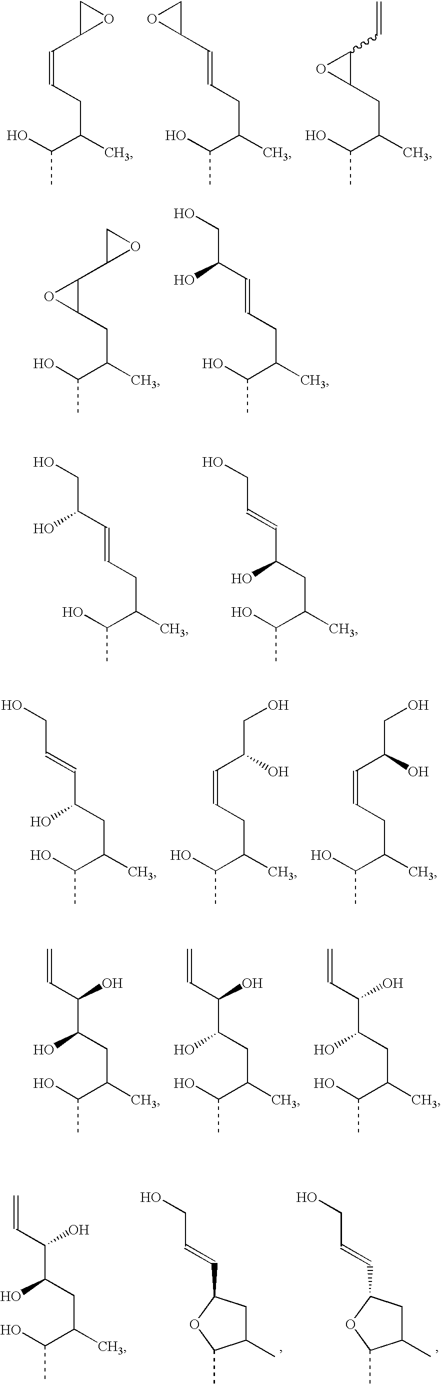 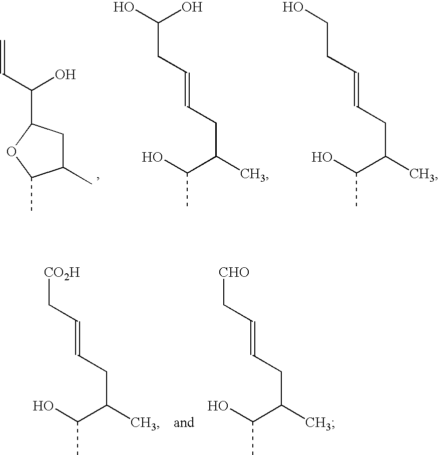
each R² is independently selected from the group consisting of —CH₃ and —H;
each R³ is independently selected from the group consisting of —CH₂CH(CH₃)₂ and —CH₂C(CH₃)₂OH; and
each R⁴ is independently selected from the group consisting of —CH(CH₃)₂ and —C(CH₃)₂OH.

4. An isolated metabolite of cyclo { {(E)- and (Z)-(2S,3R,4R)-3-hydroxy-4-methyl-2-(methylamino)-6,8-nonadienoyl}-L-2-aminobutyryl-N-methyl-glycyl-N-methyl-L- leucyl-L-valyl-N-methyl-L-leucyl-L-alanyl-D-alanyl-N-methyl-L-leucyl-N-methyl-L-leucyl-N-methyl-L-valyl} (ISA247) and pharmaceutically acceptable salts and solvates thereof, wherein compared to ISA247, the isolated metabolite comprises at least one chemical modification selected from the group consisting of hydroxylation, N-demethylation, diol formation, epoxide formation, intramolecular cyclization, phosphorylation, sulfation, glucuronide formation and glycosylation.

5. The isolated metabolite of claim 4, wherein the isolated metabolite comprises at least one chemical modification selected from the group consisting of:
an epoxide at a side chain of amino acid-1;
a diol at the side chain of amino acid 1;
a cyclic ether in the side chain of amino acid-1;
a demethylated amino nitrogen at amino acid-1, 3, 4, 6, 9, 10, or 11;
an —OH at the γ carbon of the side chain of amino acid 4, 6, 9, or 10; and
an —OH at the β carbon of the side chain of amino acid 5 or 11.

6. The isolated metabolite of claim 4, wherein the isolated metabolite is selected from the group consisting of IM1-e-1, IM1-e-2, IM1-e-3, IM1-d-1, IM1-d-2, IM1-d-3, IM1-d-4, IM1-c-1 and IM1-c-2.

7. The isolated metabolite of claim 4, wherein compared to ISA247 the isolated metabolite comprises chemical modifications selected from the group consisting of:
at least two —OH groups;
at least two demethylated amino acid nitrogens;
at least one —OH group and at least one demethylated amino acid nitrogen;
at least one diol group and at least one —OH group;
at least one diol group and at least one demethylated amino acid nitrogen;
at least one cyclic ether and at least one —OH group;
at least one cyclic ether and at least one demethylated amino acid nitrogen;
at least one —OH group and a phosphate, sulfate, glucuronide or glycosylation residue; and
at least one diol and a phosphate, sulfate, glucuronide or glycosylation residue.

8. A method of preparing metabolites of ISA247 in vitro, comprising the steps of:
a) homogenizing mammalian cells to form a homogenate;
b) centrifuging the homogenate to form a microsomal pellet, the microsomal pellet comprising at least one drug metabolizing enzyme; and
c) preparing a reaction mixture containing ISA247, the microsomal pellet, an energy source, and an electron donating species under conditions which result in production of at least one metabolite of ISA247.

9. The method of claim 8, wherein the mammalian cells are liver cells of a mammal selected from the group consisting of primate, rat, dog and rabbit.

10. The method of claim 8, wherein the drug metabolizing enzyme is a cytochrome P-450 enzyme.

11. The method of claim 8, wherein the electron donating species is selected from the group consisting of NADH and NADPH.

12. The method of claim 8, wherein the energy source is selected from the group consisting of glucose-6-phosphate and isocitrate.

13. The method of claim 12, wherein the reaction mixture further includes an enzyme selected from the group consisting of glucose-6-phosphate dehydrogenase and isocitrate dehydrogenase.

14. The method of claim 8 further comprising the step of isolating the metabolite of ISA247 using high performance liquid chromatography.

15. A method of producing a hydroxylated metabolite of ISA247, comprising the steps of:
a) protecting the β-alcohol of the 1-amino acid residue of ISA247 to form a protected-ISA247 compound;
b) halogenating the protected-ISA247 compound with a halogenating agent at the γ-carbon of the side chains of at least one of the 4, 6, or 9-amino acid residues, thereby forming a halogenated product;
c) heating the halogenated product of step b) in the presence of an acetate reagent to form an acetate-containing product having an acetate moiety; and
d) performing a transesterification to exchange the acetate moiety of the acetate-containing product of step c) with an alcohol moiety,
thereby forming the hydroxylated metabolite of ISA247.

16. The method of claim 15, wherein the halogenating agent is N-bromosuccinimide (NBS) and the acetate reagent is tetrabutylammonium acetate.

17. An isolated hydroxylated metabolite of ISA247 produced by the method of claim 15.

18. The isolated hydroxylated metabolite of claim 17, wherein the hydroxylated metabolite is selected from the group consisting of IM9, IM4, IM6, IM46, IM69 and IM49.

19. A method of producing an epoxide metabolite of ISA247 in vitro, comprising the step of oxidizing an alkene moiety of the side chain of the 1-amino acid residue of isolated ISA247 with an oxidizing agent, thereby forming the epoxide metabolite of ISA247.

20. The method of claim 19, wherein the oxidizing step is a Prilezhaev reaction.

21. The method of claim 19, wherein the oxidizing agent is selected from the group consisting of m-chloroperbenzoic acid (MCPBA), peracetic acid, trifluoroperacetic acid, perbenzoic acid, 3,5-dinitroperbenzoic acid, hydrogen peroxide, alkyl peroxide, and oxygen.

22. An isolated epoxide metabolite of ISA247 prepared by the method of claim 19.

23. The isolated epoxide metabolite of claim 22, wherein the metabolite is selected from the group consisting of IM1-e-1, IM1-e-2 and IM1-e-3.

24. A method of producing a diol metabolite of ISA247 in vitro, comprising the steps of:
a) treating an alkene moiety of the side chain of the 1-amino acid residue of ISA247 with an oxidizing agent to form a epoxide metabolite of ISA247; and
b) forming the isolated diol metabolite of ISA247 from the isolated epoxide metabolite of ISA247.

25. The method of claim 24, wherein step a) is a Prilezhaev reaction.

26. The method of claim 24, wherein the oxidizing agent is selected from the group consisting of m-chloroperbenzoic acid (MCPBA), peracetic acid, triflouroperacetic acid, perbenzoic acid, 3,5-dinitroperbenzoic acid, hydrogen peroxide, alkyl peroxide, and oxygen.

27. The method of claim 24, wherein step b) comprises hydrolyzing the epoxide metabolite of ISA247.

28. The method of claim 27, wherein the hydrolysis in step b) is catalyzed by an acid or a base.

29. The method of claim 27, wherein step b) comprises:
hydrolysis catalyzed by perchloric acid or Nafion-H;
alkaline hydrolysis in dimethyl sulfoxide; or
hydrolysis catalyzed by microsomal epoxide hydrolase.

30. An isolated diol metabolite of ISA247 prepared by the method of claim 24.

31. The isolated diol metabolite of claim 30, wherein the isolated diol metabolite is selected from the group consisting of IM1-d-1, IM1-d-2, IM1-d-3 and IM1-d-4.

32. A method of producing a diol metabolite of ISA247, comprising the step of reacting isolated ISA247 with a reagent selected from the group consisting of osmium tetroxide, alkaline potassium permanganate, hydrogen peroxide, monopersuccinic acid and t-butyl hydroperoxide, thereby forming the diol metabolite of ISA247.

33. The method of claim 32, wherein the ISA247 is reacted with a catalytic amount of osmium tetroxide.

34. The method of claim 32, wherein the ISA247 is reacted with a reagent selected from the group consisting of hydrogen peroxide/formic acid and monopersuccinic acid.

35. A method of producing a diol metabolite of ISA247, comprising the steps of:
a) treating ISA247 with a reagent selected from the group consisting of iodine/silver benzoate and silver acetate to form a diester of ISA247; and
b) hydrolyzing the diester of ISA247,
thereby forming the diol metabolite of ISA247.

36. An isolated diol metabolite of ISA247 prepared by the method of claim 35.

37. The isolated diol metabolite of claim 36, wherein the isolated diol metabolite is selected from the group consisting of IM1-d-1 and IM1-d-2.

38. A method of producing a diol metabolite of ISA247, comprising the steps of:
a) reacting ISA247 with a reagent selected from the group consisting of lead tetraacetate and thallium acetate to form a diol bisacetate of ISA247; and
b) hydrolyzing the diol bisacetate of ISA247,
thereby forming the diol metabolite of ISA247.

39. An isolated diol metabolite of ISA247 prepared by the method of claim 38.

40. The isolated diol metabolite of claim 39, wherein the isolated diol metabolite is selected from the group consisting of IM1-d-1 and IM1-d-2.

41. A pharmaceutical composition comprising a pharmaceutically acceptable carrier and the isolated compound of claim 1.

42. A pharmaceutical composition comprising a pharmaceutically acceptable carrier and the isolated metabolite of claim 2.
